SEQUENCE LISTING (114 sequences)

SEQ ID NO: 1 - protein, 1186 aa, Homo sapiens
MSVACVLKRKAVLWQDSFSPHLKHHPQEPANPNMPVVLTSGTGSQAQPQPAANQALAAGTHSSPVPGSIGVAGRSQDDAMVDYFFQRQHGEQLGGGGSGGGGYNNSKHRWPTGDNIHAEHQVRSMDELNHDFQALALEGRAMGEQLLPGKKFWETDESSKDGPKGIFLGDQWRDSAWGTSDHSVSQPIMVQRRPGQSFHVNSEVNSVLSPRSESGGLGVSMVEYVLSSSPGDSCLRKGGFGPRDADSDENDKGEKKNKGTFDGDKLGDLKEEGDVMDKTNGLPVQNGIDADVKDFSRTPGNCQNSANEVDLLGPNQNGSEGLAQLTSTNGAKPVEDFSNMESQSVPLDPMEHVGMEPLQFDYSGTQVPVDSAAATVGLFDYNSQQQLFQRPNALAVQQLTAAQQQQYALAAAHQPHIGLAPAAFVPNPYIISAAPPGTDPYTAGLAAAATLGPAVVPHQYYGVTPWGVYPASLFQQQAAAAAAATNSANQQTTPQAQQGQQQVLRGGASQRPLTPNQNQQGQQTDPLVAAAAVNSALAFGQGLAAGMPGYPVLAPAAYYDQTGALVVNAGARNGLGAPVRLVAPAPVIISSSAAQAAVAAAAASANGAAGGLAGTTNGPFRPLGTQQPQPQPQQQPNNNLASSSFYGNNSLNSNSQSSSLFSQGSAQPANTSLGFGSSSSLGATLGSALGGFGTAVANSNTGSGSRRDSLTGSSDLYKRTSSSLTPIGHSFYNGLSFSSSPGPVGMPLPSQGPGHSQTPPPSLSSHGSSSSLNLGGLTNGSGRYISAAPGAEAKYRSASSASSLFSPSSTLFSSSRLRYGMSDVMPSGRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPQSLQFIIDAFKGQVFALSTHPYGCRVIQRILEHCLPDQTLPILEELHQHTEQLVQDQYGNYVIQHVLEHGRPEDKSKIVAEIRGNVLVLSQHKFASNVVEKCVTHASRTERAVLIDEVCTMNDGPHSALYTMMKDQYANYVVQKMIDVAEPGQRKIVMHKIRPHIATLRKYTYGKHILAKLEKYYMKNGVDLGPICGPPNGII

SEQ ID NO: 2-5 - skipped (4 entries)

SEQ ID NO: 6 - protein, 412 aa, synthetic construct
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGQVFALSTHPYGCRVIQRILEHCLPDQTILEELHQHTEQLVQDQYGNYVIQHVLEHGRPEDKSKIVAEIRGNVLVLSQHKFASNVVEKCVTHASRTERAVLIDEVCTALYTMMKDQYANYVVQKMIDVAEPGQRKIVMHKIRPHTEQLVQDQYGNYVIQHVLEHGRPEDKSKIVAEIRGNVLVLSQHKFASNVVEKCVTHASRTERAVLIDEVCTALYTMMKDQYANYVVQKMIDVAEPGQRKIVMHKIRPHIATLRKYTYGKHILAKLEKYYMKNGVDLG

SEQ ID NO: 7 - protein, 412 aa, synthetic construct
GRSRLLEDFRNNRYPNLQLREIAGHIMEFSQDQHGSYFIRLKLERATPAERQLVFNEILQAAYQLMVDVFGSYVIEKFFEFGSLEQKLALAERIRGHVLSLALQMYGCRVIQKALEFIPSDQQNEMVRELDGQVFALSTHPYGSYVIRRILEHCLPDQTILEELHQHTEQLVQDQYGSYVIEHVLEHGRPEDKSKIVAEIRGNVLVLSQHKFACRVVQKCVTHASRTERAVLIDEVCTALYTMMKDQYASYVVRKMIDVAEPGQRKIVMHKIRPHTEQLVQDQYGSYVIEHVLEHGRPEDKSKIVAEIRGNVLVLSQHKFACRVVQKCVTHASRTERAVLIDEVCTALYTMMKDQYASYVVRKMIDVAEPGQRKIVMHKIRPHIATLRKYTYGKHILAKLEKYYMKNGVDLG

SEQ ID NO: 8 - protein, 412 aa, synthetic construct
GRSRLLEDFRNNRYPNLQLREIAGHIMEFSQDQHGCRFIQLKLERATPAERQLVFNEILQAAYQLMVDVFGSYVIRKFFEFGSLEQKLALAERIRGHVLSLALQMYGSYVIEKALEFIPSDQQNEMVRELDGQVFALSTHPYGCRVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHIATLRKYTYGKHILAKLEKYYMKNGVDLG

SEQ ID NO: 9 - protein, 412 aa, synthetic construct
GRSRLLEDFRNNRYPNLQLREIAGHIMEFSQDQHGSYFIELKLERATPAERQLVFNEILQAAYQLMVDVFGCRVIQKFFEFGSLEQKLALAERIRGHVLSLALQMYGSYVIRKALEFIPSDQQNEMVRELDGQVFALSTHPYGSYVIERILEHCLPDQTILEELHQHTEQLVQDQYGCRVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHTEQLVQDQYGCRVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHIATLRKYTYGKHILAKLEKYYMKNGVDLG

SEQ ID NO: 10 - protein, 340 aa, synthetic construct
GRSRLLEDFRNNRYPNLQLREIAGHIMEFSQDQHGSRFIQLKLERATPAERQLVFNEILQAAYQLMVDVFGNYVIQKFFEFGSLEQKLALAERIRGHVLSLALQMYGCRVIQKALEFIPSDQQNEMVRELDGHVLKCVKDQNGNHVVQKCIECVQPEDKSKIVAEIRGQVFALSTHPYGCRVIQRILEHCLPDQTILEELHQHTEQLVQDQYGNYVIQHVLEHGRPEDKSKIVAEIRGNVLVLSQHKFASNVVEKCVTHASRTERAVLIDEVCTALYTMMKDQYANYVVQKMIDVAEPGQRKIVMHKIRPHIATLRKYTYGKHILAKLEKYYMKNGVDLG

SEQ ID NO: 11 - protein, 340 aa, synthetic construct
GRSRLLEDFRNNRYPNLQLREIAGHIMEFSQDQHGCRFIQLKLERATPAERQLVFNEILQAAYQLMVDVFGSYVIRKFFEFGSLEQKLALAERIRGHVLSLALQMYGSYVIEKALEFIPSDQQNEMVRELDGHVLKCVKDQNGNHVVQKCIECVQPEDKSKIVAEIRGQVFALSTHPYGSYVIRRILEHCLPDQTILEELHQHTEQLVQDQYGSYVIEHVLEHGRPEDKSKIVAEIRGNVLVLSQHKFANYVVQKCVTHASRTERAVLIDEVCTALYTMMKDQYASYVVRKMIDVAEPGQRKIVMHKIRPHIATLRKYTYGKHILAKLEKYYMKNGVDLG

SEQ ID NO: 12 - protein, 340 aa, synthetic construct
GRSRLLEDFRNNRYPNLQLREIAGHIMEFSQDQHGSYFIELKLERATPAERQLVFNEILQAAYQLMVDVFGNYVIQKFFEFGSLEQKLALAERIRGHVLSLALQMYGSYVIRKALEFIPSDQQNEMVRELDGHVLKCVKDQNGSYVVEKCIECVQPEDKSKIVAEIRGQVFALSTHPYGNYVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYANYVVQKMIDVAEPGQRKIVMHKIRPHIATLRKYTYGKHILAKLEKYYMKNGVDLG

SEQ ID NO: 13 - protein, 340 aa, synthetic construct
GRSRLLEDFRNNRYPNLQLREIAGHIMEFSQDQHGSYFIRLKLERATPAERQLVFNEILQAAYQLMVDVFGSYVIEKFFEFGSLEQKLALAERIRGHVLSLALQMYGNYVIQKALEFIPSDQQNEMVRELDGHVLKCVKDQNGSYVVRKCIECVQPEDKSKIVAEIRGQVFALSTHPYGSYVIERILEHCLPDQTILEELHQHTEQLVQDQYGNYVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHIATLRKYTYGKHILAKLEKYYMKNGVDLG

SEQ ID NO: 14 - protein, 7 aa, synthetic construct
VDTGNGS

SEQ ID NO: 15 - skipped

SEQ ID NO: 16 - protein, 16 aa, synthetic construct
VDFVGYPRFPAPVEFI

SEQ ID NO: 17 - protein, 11 aa, synthetic construct
VDMALHARNIA

SEQ ID NO: 18 - protein, 13 aa, synthetic construct
VDLLALDREVQEL

SEQ ID NO: 19 - protein, 10 aa, synthetic construct
LLALDREVQE

SEQ ID NO: 20 - protein, 9 aa, synthetic construct
LLALDREVQ

SEQ ID NO: 21 - protein, 8 aa, synthetic construct
LLALDREV

SEQ ID NO: 22 - protein, 10 aa, synthetic construct
VDHIQRGGSP

SEQ ID NO: 23 - protein, 12 aa, synthetic construct
VDRRMARDGLVH

SEQ ID NO: 24 - protein, 14 aa, synthetic construct
FVGYPRFPAPVEFI

SEQ ID NO: 25 - protein, 11 aa, synthetic construct
LLALDREVQEL

SEQ ID NO: 26 - protein, 9 aa, synthetic construct
MALHARNIA

SEQ ID NO: 27 - protein, 10 aa, synthetic construct
LGHIQRGGSP

SEQ ID NO: 28 - RNA, 10 nt, Homo sapiens
  source 1..10: mol_type = mRNA
cagcagcagc

SEQ ID NO: 29 - RNA, 10 nt, Homo sapiens
  source 1..10: mol_type = mRNA
agcagcagca

SEQ ID NO: 30 - RNA, 10 nt, Homo sapiens
  source 1..10: mol_type = mRNA
gcagcagcag

SEQ ID NO: 31-33 - skipped (3 entries)

SEQ ID NO: 34 - protein, 182 aa, synthetic construct
QMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVG

SEQ ID NO: 35 - protein, 528 aa, synthetic construct
GRSRLLEDFRNNRYPNLQLREIAGHIMEFSQDQHGCRFIQLKLERATPAERQLVFNEILQAAYQLMVDVFGSYVIRKFFEFGSLEQKLALAERIRGHVLSLALQMYGSYVIEKALEFIPSDQQNEMVRELDGHVLKCVKDQNGNHVVQKCIECVQPEDKSKIVAEIRGQVFALSTHPYGSYVIRRILEHCLPDQTILEELHQHTEQLVQDQYGSYVIEHVLEHGRPEDKSKIVAEIRGNVLVLSQHKFANYVVQKCVTHASRTERAVLIDEVCTALYTMMKDQYASYVVRKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 36 - protein, 528 aa, synthetic construct
GRSRLLEDFRNNRYPNLQLREIAGHIMEFSQDQHGSYFIELKLERATPAERQLVFNEILQAAYQLMVDVFGCRVIQKFFEFGSLEQKLALAERIRGHVLSLALQMYGSYVIRKALEFIPSDQQNEMVRELDGHVLKCVKDQNGSYVVEKCIECVQPEDKSKIVAEIRGQVFALSTHPYGCRVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 37 - protein, 528 aa, synthetic construct
GRSRLLEDFRNNRYPNLQLREIAGHIMEFSQDQHGSYFIRLKLERATPAERQLVFNEILQAAYQLMVDVFGSYVIEKFFEFGSLEQKLALAERIRGHVLSLALQMYGNYVIQKALEFIPSDQQNEMVRELDGHVLKCVKDQNGSYVVRKCIECVQPEDKSKIVAEIRGQVFALSTHPYGSYVIERILEHCLPDQTILEELHQHTEQLVQDQYGNYVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 38 - protein, 554 aa, synthetic construct
MADYKDHEGDYKDHDIDYKDDDDKEFGRSRLLEDFRNNRYPNLQLREIAGHIMEFSQDQHGCRFIQLKLERATPAERQLVFNEILQAAYQLMVDVFGSYVIRKFFEFGSLEQKLALAERIRGHVLSLALQMYGSYVIEKALEFIPSDQQNEMVRELDGHVLKCVKDQNGNHVVQKCIECVQPEDKSKIVAEIRGQVFALSTHPYGSYVIRRILEHCLPDQTILEELHQHTEQLVQDQYGSYVIEHVLEHGRPEDKSKIVAEIRGNVLVLSQHKFANYVVQKCVTHASRTERAVLIDEVCTALYTMMKDQYASYVVRKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 39 - protein, 554 aa, synthetic construct
MADYKDHEGDYKDHDIDYKDDDDKEFGRSRLLEDFRNNRYPNLQLREIAGHIMEFSQDQHGSYFIELKLERATPAERQLVFNEILQAAYQLMVDVFGCRVIQKFFEFGSLEQKLALAERIRGHVLSLALQMYGSYVIRKALEFIPSDQQNEMVRELDGHVLKCVKDQNGSYVVEKCIECVQPEDKSKIVAEIRGQVFALSTHPYGCRVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 40 - protein, 554 aa, synthetic construct
MADYKDHEGDYKDHDIDYKDDDDKEFGRSRLLEDFRNNRYPNLQLREIAGHIMEFSQDQHGSYFIRLKLERATPAERQLVFNEILQAAYQLMVDVFGSYVIEKFFEFGSLEQKLALAERIRGHVLSLALQMYGNYVIQKALEFIPSDQQNEMVRELDGHVLKCVKDQNGSYVVRKCIECVQPEDKSKIVAEIRGQVFALSTHPYGSYVIERILEHCLPDQTILEELHQHTEQLVQDQYGNYVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHIATLRKYTYGKHILAKLEKYYMKNGVDLGVDTANGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 41 - protein, 181 aa, synthetic construct
QMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNDDLILSCCLHYCKDKAKDFMPASKEEPIRLLREVVLLTDDRNLRVKALTRNVPVRDIPAFLTWAQV

SEQ ID NO: 42 - protein, 7 aa, synthetic construct
VDTANGS

SEQ ID NO: 43 - skipped

SEQ ID NO: 44 - protein, 340 aa, synthetic construct
GRSRLLEDFRNNRYPNLQLREIAGHIMEFSQDQHGSYFIELKLERATPAERQLVFNEILQAAYQLMVDVFGCRVIQKFFEFGSLEQKLALAERIRGHVLSLALQMYGSYVIRKALEFIPSDQQNEMVRELDGHVLKCVKDQNGSYVVEKCIECVQPEDKSKIVAEIRGQVFALSTHPYGCRVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHIATLRKYTYGKHILAKLEKYYMKNGVDLG

SEQ ID NO: 45 - protein, 26 aa, synthetic construct
MADYKDHEGDYKDHDIDYKDDDDKEF

SEQ ID NO: 46 - protein, 601 aa, synthetic construct
GRSRLLEDFRNNRYPNLQLREIAGHIMEFSQDQHGSYFIRLKLERATPAERQLVFNEILQAAYQLMVDVFGSYVIEKFFEFGSLEQKLALAERIRGHVLSLALQMYGCRVIQKALEFIPSDQQNEMVRELDGQVFALSTHPYGSYVIRRILEHCLPDQTILEELHQHTEQLVQDQYGSYVIEHVLEHGRPEDKSKIVAEIRGNVLVLSQHKFACRVVQKCVTHASRTERAVLIDEVCTALYTMMKDQYASYVVRKMIDVAEPGQRKIVMHKIRPHTEQLVQDQYGSYVIEHVLEHGRPEDKSKIVAEIRGNVLVLSQHKFACRVVQKCVTHASRTERAVLIDEVCTALYTMMKDQYASYVVRKMIDVAEPGQRKIVMHKIRPHIATLRKYTYGKHILAKLEKYYMKNGVDLGVDTGNGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVG

SEQ ID NO: 47 - protein, 601 aa, synthetic construct
GRSRLLEDFRNNRYPNLQLREIAGHIMEFSQDQHGCRFIQLKLERATPAERQLVFNEILQAAYQLMVDVFGSYVIRKFFEFGSLEQKLALAERIRGHVLSLALQMYGSYVIEKALEFIPSDQQNEMVRELDGQVFALSTHPYGCRVIQRILEHCLPDQTILEELHQHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHTEQLVQDQYGSYVIRHVLEHGRPEDKSKIVAEIRGNVLVLSQHKFASYVVEKCVTHASRTERAVLIDEVCTALYTMMKDQYACRVVQKMIDVAEPGQRKIVMHKIRPHIATLRKYTYGKHILAKLEKYYMKNGVDLGVDTGNGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVG

SEQ ID NO: 48 - protein, 601 aa, synthetic construct
GRSRLLEDFRNNRYPNLQLREIAGHIMEFSQDQHGSYFIELKLERATPAERQLVFNEILQAAYQLMVDVFGCRVIQKFFEFGSLEQKLALAERIRGHVLSLALQMYGSYVIRKALEFIPSDQQNEMVRELDGQVFALSTHPYGSYVIERILEHCLPDQTILEELHQHTEQLVQDQYGCRVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHTEQLVQDQYGCRVIQHVLEHGRPEDKSKIVAEIRGNVLVLSQHKFASYVVRKCVTHASRTERAVLIDEVCTALYTMMKDQYASYVVEKMIDVAEPGQRKIVMHKIRPHIATLRKYTYGKHILAKLEKYYMKNGVDLGVDTGNGSQMELEIRPLFLVPDTNGFIDHLASLARLLESRKYILVVPLIVINELDGLAKGQETDHRAGGYARVVQEKARKSIEFLEQRFESRDSCLRALTSRGNELESIAFRSEDITGQLGNNADLILSCCLHYCKDKAKDFMPASKEEPIRLLREVVLLTDDRNLRVKALTRNVPVRDIPAFLTWAQVG

SEQ ID NO: 49 - protein, 128 aa, synthetic construct
KESRAKKFQRQHMDSDSSPSSSSTYCNQMMRRRNMTQGLCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 50 - protein, 119 aa, synthetic construct
QDGMYQRFLRQHVHPEETGGSDRYCDLMMQRRKMTLYHCKRFNTFIHEDIWNIRSICSTTNIQCKNGKMNCHEGVVKVTDCRDTGSSRAPNCRYRAIASTRRVVIACEGNPQVPVHFDG

SEQ ID NO: 51 - protein, 127 aa, synthetic construct
WPKRLTKAHWFEIQHIQPSPLQCNRAMSGINNYTQHCKHQNTFLHDSFQNVAAVCDLLSIVCKNRRHNCHQSSKPVNMTDCRLTSGKYPQCRYSAAAQYKFFIVACDPPQKSDPPYKLVPVHLDSIL

SEQ ID NO: 52 - protein, 155 aa, synthetic construct
APARAGFCPLLLLLLLGLWVAEIPVSAKPKGMTSSQWFKIQHMQPSPQACNSAMKNINKHTKRCKDLNTFLHEPFSSVAATCQTPKIACKNGDKNCHQSHGPVSLTMCKLTSGKYPNCRYKEKRQNKSYVVACKPPQKKDSQQFHLVPVHLDRVL

SEQ ID NO: 53 - protein, 155 aa, synthetic construct; identical to SEQ ID NO: 52

SEQ ID NO: 54 - protein, 134 aa, synthetic construct
KPPQFTWAQWFETQHINMTSQQCTNAMQVINNYQRRCKNQNTFLLTTFANVVNVCGNPNMTCPSNKTRKNCHHSGSQVPLIHCNLTTPSPQNISNCRYAQTPANMFYIVACDNRDQRRDPPQYPVVPVHLDRII

SEQ ID NO: 55 - protein, 216 aa, synthetic construct
DKRLRDNHEWKKLIMVQHWPETVCEKIQNDCRDPPDYWTIHGLWPDKSEGCNRSWPFNLEEIKKNWMEITDSSLPSPSMGPAPPRWMRSTPRRSTLAEAWNSTGSWTSTGGCALPPAALPSGDLCCRPSLTAGSRGVGVDLTALHQLLHVHYSATGIIPEECSEPTKPFQIILHHDHTEWVQSIGMPIWGTISSSESAIGKNEESQPACAVLSHDS

SEQ ID NO: 56 - protein, 722 aa, synthetic construct
AAVEDNHLLIKAVQNEDVDLVQQLLEGGANVNFQEEEGGWTPLHNAVQMSREDIVELLLRHGADPVLRKKNGATPFILAAIAGSVKDLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 57 - protein, 217 aa, synthetic construct
VQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 58 - protein, 183 aa, synthetic construct
EASESTMKIIKEEFTDEEMQYDMAKSGQEKQTIEILMNPILLVKNTSLSMSKDDMSSTLLTFRSLHYNDPKGNSSGNDKECCNDMTVWRKVSEANGSCKWSNNFIRSSTEVMRRVHRAPSCKFVQNPGISCCESLELENTVCQFTTGKQFPRCQYHSVTSLEKILTVLTGHSLMSWLVCGSKL

SEQ ID NO: 59 - protein, 185 aa, synthetic construct
  VARIANT 1: note = Any amino acid
XLGGADKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQTTEEDLNLDVEPTTEDTAEEVTIHVLLHSALFGEIGPRRW

SEQ ID NO: 60 - protein, 128 aa, synthetic construct
KESRAKKFQRQHMDSDSSPSSSSTYCNQMMRRRNMTQGRCRPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 61 - protein, 128 aa, synthetic construct
KESRAKKFQRQHMDSDSSPSSSSTYCNQMMRRRNMTQGRCRPVNTFVHEPLVDVQNVCFQEKVTCKNGQGNCYKSNSSMHITDCRLTNGSRYPNCAYRTSPKERHIIVACEGSPYVPVHFEASVEDST

SEQ ID NO: 62 - protein, 128 aa, synthetic construct
KESRAKKFQRQHMDSDSSPSSSSTYCNQMMRRRNMTQGRCRPVNTFVHEPLVDVQNVCFQEKVTCKNGQGNCYKSNSSMHITDCRLTNGSRYPNCAYRTSPKERHIIVACEGSPYVPVNFEASVEDST

SEQ ID NO: 63 - protein, 128 aa, synthetic construct
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVNFDASVEDST

SEQ ID NO: 64 - protein, 128 aa, synthetic construct
KESRAKKFQRQHMDSDSSPSSSSTYCNQMMRRRNMTQGDCKPVNTFVHEPLVDVQNVCFQEKVTCKDGQGNCYKSNSSMHITDCRLTADSDYPNCAYRTSPKERHIIVACEGSPYVPVNFDASVEDST

SEQ ID NO: 65 - protein, 128 aa, synthetic construct
KESRAKKFQRQHMDSDSSPSSSSTYCNQMMRRRNMTQGDCRPVNTFVHEPLVDVQNVCFQEKVTCKDGQGNCYKSNSSMHITDCRLTADSDYPNCAYRTSPKERHIIVACEGSPYVPVNFEASVEDST

SEQ ID NO: 66 - protein, 128 aa, synthetic construct
KESRAKKFQRQHMDSDSSPSSSSTYCNQMMRRRNMTQGDCKPVNTFVHEPLVDVQNVCFQEKVTCKDGQGNCYKSNSSMHITDCRLTADSDYPNCAYRTSPKERHIIVACEGSPYVPVHFDASVEDST

SEQ ID NO: 67 - protein, 128 aa, synthetic construct; identical to SEQ ID NO: 65

SEQ ID NO: 68 - protein, 299 aa, synthetic construct
APVEHVVADAGAFLRHAALQDIGKNIYTIREVVTEIRDKATRRRLAVLPYELRFKEPLPEYVRLVTEFSKKTGDYPSLSATDIQVLALTYQLEAEFVGVSHLKQEPQKVKVSSSIQHPETPLHISGFHLPYKPKPPQETEKGHSACEPENLEFSSFMFWRNPLPNIDHELQELLIDRGEDVPSEEEEEEENGFEDRKDDSDDDGGGWITPSNIKQIQQELEQCDVPEDVRVGCLTTDFAMQNVLLQMGLHVLAVNGMLIREARSYILRCHGCFKTTSDMSRVFCSHCGNKTLKKVSVTV

SEQ ID NO: 69 - protein, 210 aa, synthetic construct
AFSGLQRVGGVDVSFVKGDSVRACASLVVLSFPELEVVYEESRMVSLTAPYVSGFLAFREVPFLLELVQQLREKEPGLMPQVLLVDGNGVLHHRGFGVACHLGVLTDLPCVGVAKKLLQVDGLENNALHKEKIRLLQTRGDSFPLLGDSGTVLGMALRSHDRSTRPLYISVGHRMSLEAAVRLTCCCCRFRIPEPVRQADICSREHIRKS

SEQ ID NO: 70 - protein, 249 aa, synthetic construct
AELPPVPGGPRGPGELAKYGLPGLAQLKSRESYVLCYDPRTRGALWVVEQLRPERLRGDGDRRECDFREDDSVHAYHRATNADYRGSGFDRGHLAAAANHRWSQKAMDDTFYLSNVAPQVPHLNQNAWNNLEKYSRSLTRSYQNVYVCTGPLFLPRTEADGKSYVKYQVIGKNHVAVPTHFFKVLILEAAGGQIELRTYVMPNAPVDEAIPLERFLVPIESIERASGLLFVPNILARAGSLKAITAGSK

SEQ ID NO: 71 - protein, 479 aa, synthetic construct
RLVGEEEAGFGECDKFFYAGTPPAGLAADSHVKICQRAEGAERFATLYSTRDRIPVYSAFRAPRPAPGGAEQRWLVEPQIDDPNSNLEEAINEAEAITSVNSLGSKQALNTDYLDSDYQRGQLYPFSLSSDVQVATFTLTNSAPMTQSFQERWYVNLHSLMDRALTPQCGSGEDLYILTGTVPSDYRVKDKVAVPEFVWLAACCAVPGGGWAMGFVKHTRDSDIIEDVMVKDLQKLLPFNPQLFQNNCGETEQDTEKMKKILEVVNQIQDEERMVQSQKSSSPLSSTRSKRSTLLPPEASEGSSSFLGKLMGFIATPFIKLFQLIYYLVVAILKNIVYFLWCVTKQVINGIESCLYRLGSATISYFMAIGEELVSIPWKVLKVVAKVIRALLRILCCLLKAICRVLSIPVRVLVDVATFPVYTMGAIPIVCKDIALGLGGTVSLLFDTAFGTLGGLFQVVFSVCKRIGYKVTFDNSGEL

SEQ ID NO: 72 - protein, 380 aa, synthetic construct
MGIQGLAKLIADVAPSAIRENDIKSYFGRKVAIDASMSIYQFLIAVRQGGDVLQNEEGETTSHLMGMFYRTIRMMENGIKPVYVFDGKPPQLKSGELAKRSERRAEAEKQLQQAQAAGAEQEVEKFTKRLVKVTKQHNDECKHLLSLMGIPYLDAPSEAEASCAALVKAGKVYAAATEDMDCLTFGSPVLMRHLTASEAKKLPIQEFHLSRILQELGLNQEQFVDLCILLGSDYCESIRGIGPKRAVDLIQKHKSIEEIVRRLDPNKYPVPENWLHKEAHQLFLEPEVLDPESVELKWSEPNEEELIKFMCGEKQFSEERIRSGVKRLSKSRQGSTQGRLDDFFKVTGSLSSAKRKEPEPKGSTKKKAKTGAAGKFKRGK

SEQ ID NO: 73 - protein, 372 aa, synthetic construct
MESGQPARRIAMAPLLEYERQLVLELLDTDGLVVCARGLGADRLLYHFLQLHCHPACLVLVLNTQPAEEEYFINQLKIEGVEHLPRRVTNEITSNSRYEVYTQGGVIFATSRILVVDFLTDRIPSDLITGILVYRAHRIIESCQEAFILRLFRQKNKRGFIKAFTDNAVAFDTGFCHVERVMRNLFVRKLYLWPRFHVAVNSFLEQHKPEVVEIHVSMTPTMLAIQTAILDILNACLKELKCHNPSLEVEDLSLENAIGKPFDKTIRHYLDPLWHQLGAKTKSLVQDLKILRTLLQYLSQYDCVTFLNLLESLRATEKAFGQNSGWLFLDSSTSMFINARARVYHLPDAKMSKKEKISEKMEIKEGEGILWG

SEQ ID NO: 74 - protein, 311 aa, synthetic construct
CSPQESGMTALSARMLTRSRSLGPGAGPRGCREEPGPLRRREAAAEARKSHSPVKRPRKAQRLRVAYEGSDSEKGEGAEPLKVPVWEPQDWQQQLVNIRAMRNKKDAPVDHLGTEHCYDSSAPPKVRRYQVLLSLMLSSQTKDQVTAGAMQRLRARGLTVDSILQTDDATLGKLIYPVGFWRSKVKYIKQTSAILQQHYGGDIPASVAELVALPGVGPKMAHLAMAVAWGTVSGIAVDTHVHRIANRLRWTKKATKSPEETRAALEEWLPRELWHEINGLLVGFGQQTCLPVHPRCHACLNQALCPAAQGL

SEQ ID NO: 75 - protein, 186 aa, synthetic construct
ESTHVEFKRFTTKKVIPRIKEMLPHYVSAFANTQGGYVLIGVDDKSKEVVGCKWEKVNPDLLKKEIENCIEKLPTFHFCCEKPKVNFTTKILNVYQKDVLDGYVCVIQVEPFCCVVFAEAPDSWIMKDNSVTRLTAEQWVVMMLDTQSAPPSLVTDYNSCLISSASSARKSPGYPIKVHKFKEALQ

SEQ ID NO: 76 - protein, 262 aa, synthetic construct
TLQGTNTYLVGTGPRRILIDTGEPAIPEYISCLKQALTEFNTAIQEIVVTHWHRDHSGGIGDICKSINNDTTYCIKKLPRNPQREEIIGNGEQQYVYLKDGDVIKTEGATLRVLYTPGHTDDHMALLLEEENAIFSGDCILGEGTTVFEDLYDYMNSLKELLKIKADIIYPGHGPVIHNAEAKIQQYISHRNIREQQILTLFRENFEKSFTVMELVKIIYKNTPENLHEMAKHNLLLHLKKLEKEGKIFSNTDPDKKWKAHL

SEQ ID NO: 77 - protein, 518 aa, synthetic construct
MLRVVSWNINGIRRPLQGVANQEPSNCAAVAVGRILDELDADIVCLQETKVTRDALTEPLAIVEGYNSYFSFSRNRSGYSGVATFCKDNATPVAAEEGLSGLFATQNGDVGCYGNMDEFTQEELRALDSEGRALLTQHKIRTWEGKEKTLTLINVYCPHADPGRPERLVFKMRFYRLLQIRAEALLAAGSHVIILGDLNTAHRPIDHWDAVNLECFEEDPGRKWMDSLLSNLGCQSASHVGPFIDSYRCFQPKQEGAFTCWSAVTGARHLNYGSRLDYVLGDRTLVIDTFQASFLLPEVMGSDHCPVGAVLSVSSVPAKQCPPLCTRFLPEFAGTQLKILRFLVPLEQSPVLEQSTLQHNNQTRVQTCQNKAQVRSTRPQPSQVGSSRGQKNLKSYFQPSPSCPQASPDIELPSLPLMSALMTPKTPEEKAVAKVVKGQAKTSEAKDEKELRTSFWKSVLAGPLRTPLCGGHREPCVMRTVKKPGPNLGRRFYMCARPRGPPTDPSSRCNFFLWSRPS

SEQ ID NO: 78 - protein, 350 aa, synthetic construct
MLRVVSWNINGIRRPLQGVANQEPSNCAAVAVGRILDELDADIVCLQETKVTRDALTEPLAIVEGYNSYFSFSRNRSGYSGVATFCKDNATPVAAEEGLSGLFATQNGDVGCYGNMDEFTQEELRALDSEGRALLTQHKIRTWEGKEKTLTLINVYCPHADPGRPERLVFKMRFYRLLQIRAEALLAAGSHVIILGDLNTAHRPIDHWDAVNLECFEEDPGRKWMDSLLSNLGCQSASHVGPFIDSYRCFQPKQEGAFTCWSAVTGARHLNYGSRLDYVLGDRTLVIDTFQASFLLPEVMGSDHCPVGAVLSVSSVPAKQCPPLCTRFLPEFAGTQLKILRFLVPLEQSP

SEQ ID NO: 79 - protein, 123 aa, synthetic construct [sequence omitted]

SEQ ID NO: 80 - protein, 136 aa, synthetic construct
SSLIRRVISTAKAPGAIGPYSQAVLVDRTIYISGQIGMDPSSGQLVSGGVAEEAKQALKNMGEILKAAGCDFTNVVKTTVLLADINDFNTVNEIYKQYFKSNFPARAAYQVAALPKGSRIEIEAVAIQGPLTTASL

SEQ ID NO: 81 - protein, 189 aa, synthetic construct
GGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLE

SEQ ID NO: 82 - protein, 598 aa, synthetic construct
SGPCGEKPVLEASPTMSLWEFEDSHSRQGTPRPGQELAAEEASALELQMKVDFFRKLGYSSTEIHSVLQKLGVQADTNTVLGELVKHGTATERERQTSPDPCPQLPLVPRGGGTPKAPNLEPPLPEEEKEGSDLRPVVIDGSNVAMSHGNKEVFSCRGILLAVNWFLERGHTDITVFVPSWRKEQPRPDVPITDQHILRELEKKKILVFTPSRRVGGKRVVCYDDRFIVKLAYESDGIVVSNDTYRDLQGERQEWKRFIEERLLMYSFVNDKFMPPDDPLGRHGPSLDNFLRKKPLTLEHRKQPCPYGRKCTYGIKCRFFHPERPSCPQRSVADELRANALLSPPRAPSKDKNGRRPSPSSQSSSLLTESEQCSLDGKKLGAQASPGSRQEGLTQTYAPSGRSLAPSGGSGSSFGPTDWLPQTLDSLPYVSQDCLDSGIGSLESQMSELWGVRGGGPGEPGPPRAPYTGYSPYGSELPATAAFSAFGRAMGAGHFSVPADYPPAPPAFPPREYWSEPYPLPPPTSVLQEPPVQSPGAGRSPWGRAGSLAKEQASVYTKLCGVFPPHLVEAVMGRFPQLLDPQQLAAEILSYKSQHPSE

SEQ ID NO: 83 - protein, 287 aa, synthetic construct
PKRGKKGAVAEDGDELRTEPEAKKSKTAAKKNDKEAAGEGPALYEDPPDQKTSPSGKPATLKICSWNVDGLRAWIKKKGLDWVKEEAPDILCLQETKCSENKLPAELQELPGLSHQYWSAPSDKEGYSGVGLLSRQCPLKVSYGIGDEEHDQEGRVIVAEFDSFVLVTAYVPNAGRGLVRLEYRQRWDEAFRKFLKGLASRKPLVLCGDLNVAHEEIDLRNPKGNKKNAGFTPQERQGFGELLQAVPLADSFRHLYPNTPYAYTFWTYMMNARSKNVGWRLDYFLLS

SEQ ID NO: 84 - protein, 213 aa, synthetic construct
EALFFPSQVTCTEALLRAPGAELAELPEGCPCGLPHGESALSRLLRALLAARASLDLCLFAFSSPQLGRAVQLLHQRGVRVRVVTDCDYMALNGSQIGLLRKAGIQVRHDQDPGYMHHKFAIVDKRVLITGSLNWTTQAIQNNRENVLITEDDEYVRLFLEEFERIWEQFNPTKYTFFPPKKSHGSCAPPVSRAGGRLLSWHRTCGTSSESQT

SEQ ID NO: 85 - protein, 382 aa, synthetic construct
KARYKTLEPRGYSLLIRGLIHSDRWREALLLLEDIKKVITPSKKNYNDCIQGALLHQDVNTAWNLYQELLGHDIVPMLETLKAFFDFGKDIKDDNYSNKLLDILSYLRNNQLYPGESFAHSIKTWFESVPGKQWKGQFTTVRKSGQCSGCGKTIESIQLSPEEYECLKGKIMRDVIDGGDQYRKTTPQELKRFENFIKSRPPFDVVIDGLNVAKMFPKVRESQLLLNVVSQLAKRNLRLLVLGRKHMLRRSSQWSRDEMEEVQKQASCFFADDISEDDPFLLYATLHSGNHCRFITRDLMRDHKACLPDAKTQRLFFKWQQGHQLAIVNRFPGSKLTFQRILSYDTVVQTTGDSWHIPYDEDLVERCSCEVPTKWLCLHQKT

SEQ ID NO: 86 - protein, 360 aa, synthetic construct
SVEPMFRHLKNTYAGLQLVVVILPGKTPVYAEVKRVGDTVLGMATQCVQMKNVQRTTPQTLSNLCLKINVKLGGVNNILLPQGRPPVFQQPVIFLGADVTHPPAGDGKKPSIAAVVGSMDAHPNRYCATVRVQQHRQEIIQDLAAMVRELLIQFYKSTRFKPTRIIFYRDGVSEGQFQQVLHHELLAIREACIKLEKDYQPGITFIVVQKRHHTRLFCTDKNERVGKSGNIPAGTTVDTKITHPTEFDFYLCSHAGIQGTSRPSHYHVLWDDNRFSSDELQILTYQLCHTYVRCTRSVSIPAPAYYAHLVAFRARYHLVDKEHDSAEGSHTSGQSNGRDHQALAKAVQVHQDTLRTMYFA

SEQ ID NO: 87 - protein, 327 aa, synthetic construct
QGAEGALTGKQPDGSAEKAVLEQFGFPLTGTEARCYTNHALSYDQAKRVPRWVLEHISKSKIMGDADRKHCKFKPDPNIPPTFSAFNEDYVGSGWSRGHMAPAGNNKFSSKAMAETFYLSNIVPQDFDNNSGYWNRIEMYCRELTERFEDVWVVSGPLTLPQTRGDGKKIVSYQVIGEDNVAVPSHLYKVILARRSSVSTEPLALGAFVVPNEAIGFQPQLTEFQVSLQDLEKLSGLVFFPHLDRTSDIRNICSVDTCKLLDFQEFTLYLSTRKIEGARSVLRLEKIMENLKNAEIEPDDYFMSRYEKKLEELKAKEQSGTQIRKPS

SEQ ID NO: 88 - protein, 526 aa, synthetic construct
EHPSKMEFFQKLGYDREDVLRVLGKLGEGALVNDVLQELIRTGSRPGALEHPAAPRLVPRGSCGVPDSAQRGPGTALEEDFRTLASSLRPIVIDGSNVAMSHGNKETFSCRGIKLAVDWFRDRGHTYIKVFVPSWRKDPPRADTPIREQHVLAELERQAVLVYTPSRKVHGKRLVCYDDRYIVKVAYEQDGVIVSNDNYRDLQSENPEWKWFIEQRLLMFSFVNDRFMPPDDPLGRHGPSLSNFLSRKPKPPEPSWQHCPYGKKCTYGIKCKFYHPERPHHAQLAVADELRAKTGARPGAGAEEQRPPRAPGGSAGARAAPREPFAHSLPPARGSPDLAALRGSFSRLAFSDDLGPLGPPLPVPACSLTPRLGGPDWVSAGGRVPGPLSLPSPESQFSPGDLPPPPGLQLQPRGEHRPRDLHGDLLSPRRPPDDPWARPPRSDRFPGRSVWAEPAWGDGATGGLSVYATEDDEGDARARARIALYSVFPRDQVDRVMAAFPELSDLARLILLVQRCQSAGAPLGKP

SEQ ID NO: 89 - protein, 475 aa, synthetic construct
RQQQPQVVEKQQETPLAPADFAHISQDAQSLHSGASRRSQKRLQSPSKQAQPLDDPEAEQLTVVGKISFNPKDVLGRGAGGTFVFRGQFEGRAVAVKRLLRECFGLVRREVQLLQESDRHPNVLRYFCTERGPQFHYIALELCRASLQEYVENPDLDRGGLEPEVVLQQLMSGLAHLHSLHIVHRDLKPGNILITGPDSQGLGRVVLSDFGLCKKLPAGRCSFSLHSGIPGTEGWMAPELLQLLPPDSPTSAVDIFSAGCVFYYVLSGGSHPFGDSLYRQANILTGAPCLAHLEEEVHDKVVARDLVGAMLSPLPQPRPSAPQVLAHPFFWSRAKQLQFFQDVSDWLEKESEQEPLVRALEAGGCAVVRDNWHEHISMPLQTDLRKFRSYKGTSVRDLLRAVRNKKHHYRELPVEVRQALGQVPDGFVQYFTNRFPRLLLHTHRAMRSCASESLFLPYYPPDSEARRPCPGATGR

SEQ ID NO: 90 - protein, 384 aa, synthetic construct
KLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYLFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEED

SEQ ID NO: 91 - protein, 166 aa, synthetic construct
SLVIRNLQRVIPIRRAPLRSKIEIVRRILGVQKFDLGIICVDNKNIQHINRIYRDRNVPTDVLSFPFHEHLKAGEFPQPDFPDDYNLGDIFLGVEYIFHQCKENEDYNDVLTVTATHGLCHLLGFTHGTEAEWQQMFQKEKAVLDELGRRTGTRLQPLTRGLFGGS

SEQ ID NO: 92 - protein, 178 aa, synthetic construct
QEVIAGLERFTFAFEKDVEMQKGTGLLPFQGMDKSASAVCNFFTKGLCEKGKLCPFRHDRGEKMVVCKHWLRGLCKKGDHCKFLHQYDLTRMPECYFYSKFGDCSNKECSFLHVKPAFKSQDCPWYDQGFCKDGPLCKYRHVPRIMCLNYLVGFCPEGPKCQFAQKIREFKLLPGSKI

SEQ ID NO: 93 - protein, 384 aa, synthetic construct
KLVRKNIEKDNAGQVTLVPEEPEDMWHTYNLVQVGDSLRASTIRKVQTESSTGSVGSNRVRTTLTLCVEAIDFDSQACQLRVKGTNIQENEYVKMGAYHTIELEPNRQFTLAKKQWDSVVLERIEQACDPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEED

SEQ ID NO: 94 - protein, 256 aa, synthetic construct
DPAWSADVAAVVMQEGLAHICLVTPSMTLTRAKVEVNIPRKRKGNCSQHDRALERFYEQVVQAIQRHIHFDVVKCILVASPGFVREQFCDYMFQQAVKTDNKLLLENRSKFLQVHASSGHKYSLKEALCDPTVASRLSDTKAAGEVKALDDFYKMLQHEPDRAFYGLKQVEKANEAMAIDTLLISDELFRHQDVATRSRYVRLVDSVKENAGTVRIFSSLHVSGEQLSQLTGVAAILRFPVPELSDQEGDSSSEED

SEQ ID NO: 95 - protein, 209 aa, synthetic construct
MDPGKDKEGVPQPSGPPARKKFVIPLDEDEVPPGVRGNPVLKFVRNVPWEFGDVIPDYVLGQSTCALFLSLRYHNLHPDYIHGRLQSLGKNFALRVLLVQVDVKDPQQALKELAKMCILADCTLILAWSPEEAGRYLETYKAYEQKPADLLMEKLEQDFVSRVTECLTTVKSVNKTDSQTLLTTFGSLEQLIAASREDLALCPGLGPQK

SEQ ID NO: 96 - protein, 128 aa, synthetic construct
KESRAKKFQRQHMDSDSSPSSSSTYCNQMMRRRNMTQGRCKPVNTFVHEPLVDVQNVCFQEKVTCKNGQGNCYKSNSSMHITDCRLTNGSRYPNCAYRTSPKERHIIVACEGSPYVPVHFDASVEDST

SEQ ID NO: 97 - protein, 123 aa, synthetic construct [sequence omitted]

SEQ ID NO: 98 - protein, 129 aa, synthetic construct
GLGLVQPSYGQDGMYQRFLRQHVHPEETGGSDRYCNLMMQRRKMTLYHCKRFNTFIHEDIWNIRSICSTTNIQCKNGKMNCHEGVVKVTDCRDTGSSRAPNCRYRAIASTRRVVIACEGNPQVPVHFDG

SEQ ID NO: 99 - protein, 375 aa, synthetic construct
  VARIANT 1: note = Any amino acid
XSAVDNILLKLAKFKIGFLRLGQIQKVHPAIQQFTEQEICRSKSIKSLALLEELYNSQLIVATTCMGINHPIFSRKIFDFCIVDEASQISQPICLGPLFFSRRFVLVGDHQQLPPLVLNREARALGMSESLFKRLEQNKSAVVQLTVQYRMNSKIMSLSNKLTYEGKLECGSDKVANAVINLRHFKDVKLELEFYADYSDNPWLMGVFEPNNPVCFLNTDKVPAPEQVEKGGVSNVTEAKLIVFLTSIFVKAGCSPSDIGIIAPYRQQLKIINDLLARSIGMVEVNTVDKYQGRDKSIVLVSFVRSNKDGTVGELLKDWRRLNVAITRAKHKLILLGCVPSLNCYPPLEKLLNHLNSEKLISFFFCIWSHLIALL

SEQ ID NO: 100 - protein, 88 aa, synthetic construct
MALRSHDRSTRPLYISVGHRMSLEAAVRLTCCCCRFRIPEPVRQADICSREHIRKSLGLPGPPTPRSPKAQRPVACPKGDSGESSALC

SEQ ID NO: 101 - protein, 212 aa, synthetic construct
CYTNHALSYDQAKRVPRWVLEHISKSKIMGDADRKHCKFKPDPNIPPTFSAFNEDYVGSGWSRGHMAPAGNNKFSSKAMAETFYLSNIVPQDFDNNSGYWNRIEMYCRELTERFEDVWVVSGPLTLPQTRGDGKKIVSYQVIGEDNVAVPSHLYKVILARRSSVSTEPLALGAFVVPNEAIGFQPQLTEFQVSLQDLEKLSGLVFFPHLDRT

SEQ ID NO: 102 - protein, 123 aa, synthetic construct
VTVSQMTSVLNGKTRRFADIQLQHGALCFNIRYGTTVEEEKNHVLEIARQRAVAQAWTKEQRRLQEGEEGIRAWTEGEKQQLLSTGRVQGYDGYFVLSVEQYLELSDSANNIHFMRQSEIGRR

SEQ ID NO: 103 - protein, 125 aa, synthetic construct
TVSQPTLLVNGKTRRFTNIEFQYSTLLLSIRYGLTPDTLDEEKARVLDQARQRALGTAWAKEQQKARDGREGSRLWTEGEKQQLLSTGRVQGYEGYYVLPVEQYPELADSSSNIQFLRQNEMGKR

SEQ ID NO: 104 - protein, 137 aa, synthetic construct
MGWLRPGPRPLCPPARASWAFSHRFPSPLAPRRSPTPFFMASLLCCGPKLAACGIVLSAWGVIMLIMLGIFFNVHSAVLIEDVPFTEKDFENGPQNIYNLYEQVSYNCFIAAGLYLLLGGFSFCQVRLNKRKEYMVR

SEQ ID NO: 105 - protein, 1245 aa, synthetic construct
MRIGKSSGWLNESVSLEYEHVSPPTRPRDTRRRPRAAGDGGLAHLHRRLAVGYAEDTPRTEARSPAPRRPLPVAPASAPPAPSLVPEPPMPVSLPAVSSPRFSAGSSAAITDPFPSLPPTPVLYAMARELEALSDATWQPAVPLPAEPPTDARRGNTVFDEASASSPVIASACPQAFASPPRAPRSARARRARTGGDAWPAPTFLSRPSSSRIGRDVFGKLVALGYSREQIRKLKQESLSEIAKYHTTLTGQGFTHADICRISRRRQSLRVVARNYPELAAALPELTRAHIVDIARQRSGDLALQALLPVATALTAAPLRLSASQIATVAQYGERPAIQALYRLRRKLTRAPLHLTPQQVVAIASNTGGKRALEAVCVQLPVLRAAPYRLSTEQVVAIASNKGGKQALEAVKAHLLDLLGAPYVLDTEQVVAIASHNGGKQALEAVKADLLDLRGAPYALSTEQVVAIASHNGGKQALEAVKADLLELRGAPYALSTEQVVAIASHNGGKQALEAVKAHLLDLRGVPYALSTEQVVAIASHNGGKQALEAVKAQLLDLRGAPYALSTAQVVAIASNGGGKQALEGIGEQLLKLRTAPYGLSTEQVVAIASHDGGKQALEAVGAQLVALRAAPYALSTEQVVAIASNKGGKQALEAVKAQLLELRGAPYALSTAQVVAIASHDGGNQALEAVGTQLVALRAAPYALSTEQVVAIASHDGGKQALEAVGAQLVALRAAPYALNTEQVVAIASSHGGKQALEAVRALFPDLRAAPYALSTAQLVAIASNPGGKQALEAVRALFRELRAAPYALSTEQVVAIASNHGGKQALEAVRALFRGLRAAPYGLSTAQVVAIASSNGGKQALEAVWALLPVLRATPYDLNTAQIVAIASHDGGKPALEAVWAKLPVLRGAPYALSTAQVVAIACISGQQALEAIEAHMPTLRQASHSLSPERVAAIACIGGRSAVEAVRQGLPVKAIRRIRREKAPVAGPPPASLGPTPQELVAVLHFFRAHQQPRQAFVDALAAFQATRPALLRLLSSVGVTEIEALGGTIPDATERWQRLLGRLGFRPATGAAAPSPDSLQGFAQSLERTLGSPGMAGQSACSPHRKRPAETAIAPRSIRRSPNNAGQPSEPWPDQLAWLQRRKRTARSHIRADSAASVPANLHLGTRAQFTPDRLRAEPGPIMQAHTSPASVSFGSHVAFEPGLPDPGTPTSADLASFEAEPFGVGPLDFHLDWLLQILET

SEQ ID NO: 106 - protein, 1373 aa, synthetic construct
MDPIRSRTPSPARELLPGPQPDRVQPTADRGGAPPAGGPLDGLPARRTMSRTRLPSPPAPSPAFSAGSFSDLLRQFDPSLLDTSLLDSMPAVGTPHTAAAPAECDEVQSGLRAADDPPPTVRVAVTAARPPRAKPAPRRRAAQPSDASPAAQVDLRTLGYSQQQQEKIKPKVGSTVAQHHEALVGHGFTHAHIVALSRHPAALGTVAVKYQDMIAALPEATHEDIVGVGKQWSGARALEALLTVAGELRGPPLQLDTGQLVKIAKRGGVTAVEAVHASRNALTGAPLNLTPAQVVAIASNNGGKQALETVQRLLPVLCQAHGLTPAQVVAIASHDGGKQALETMQRLLPVLCQAHGLPPDQVVAIASNIGGKQALETVQRLLPVLCQAHGLTPDQVVAIASHGGGKQALETVQRLLPVLCQAHGLTPDQVVAIASHDGGKQALETVQRLLPVLCQAHGLTPDQVVAIASNGGGKQALETVQRLLPVLCQAHGLTPDQVVAIASNGGKQALETVQRLLPVLCQAHGLTPDQVVAIASHDGGKQALETVQRLLPVLCQTHGLTPAQVVAIASHDGGKQALETVQQLLPVLCQAHGLTPDQVVAIASNIGGKQALATVQRLLPVLCQAHGLTPDQVVAIASNGGGKQALETVQRLLPVLCQAHGLTPDQVVAIASNGGGKQALETVQRLLPVLCQAHGLTQVQVVAIASNIGGKQALETVQRLLPVLCQAHGLTPAQVVAIASHDGGKQALETVQRLLPVLCQAHGLTPDQVVAIASNGGGKQALETVQRLLPVLCQAHGLTQEQVVAIASNNGGKQALETVQRLLPVLCQAHGLTPDQVVAIASNGGGKQALETVQRLLPVLCQAHGLTPAQVVAIASNIGGKQALETVQRLLPVLCQDHGLTLAQVVAIASNIGGKQALETVQRLLPVLCQAHGLTQDQVVAIASNIGGKQALETVQRLLPVLCQDHGLTPDQVVAIASNIGGKQALETVQRLLPVLCQDHGLTLDQVVAIASNGGKQALETVQRLLPVLCQDHGLTPDQVVAIASNSGGKQALETVQRLLPVLCQDHGLTPNQVVAIASNGGKQALESIVAQLSRPDPALAALTNDHLVALACLGGRPAMDAVKKGLPHAPELIRRVNRRIGERTSHRVADYAQVVRVLEFFQCHSHPAYAFDEAMTQFGMSRNGLVQLFRRVGVTELEARGGTLPPASQRWDRILQASGMKRAKPSPTSAQTPDQASLHAFADSLERDLDAPSPMHEGDQTGASSRKRSRSDRAVTGPSAQHSFEVRVPEQRDALHLPLSWRVKRPRTRIGGGLPDPGTPIAADLAASSTVMWEQDAAPFAGAADDFPAFNEEELAWLMELLPQSGSVGGTI

SEQ ID NO: 107 - protein, 1978 aa, synthetic construct
MSRPRFNPRGDFPLQRPRAPNPSGMRPPGPFMRPGSMGLPRFYPAGRARGIPHRFAGHESYQNMGPQRMNVQVTQHRTDPRLTKEKLDFHEAQQKKGKPHGSRWDDEPHISASVAVKQSSVTQVTEQSPKVQSRYTKESASSILASFGLSNEDLEELSRYPDEQLTPENMPLILRDIRMRKMGRRLPNLPSQSRNKETLGSEAVSSNVIDYGHASKYGYTEDPLEVRIYDPEIPTDEVENEFQSQQNISASVPNPNVICNSMFPVEDVFRQMDFPGESSNNRSFFSVESGTKMSGLHISGGQSVLEPIKSVNQSINQTVSQTMSQSLIPPSMNQQPFSSELISSVSQQERIPHEPVINSSNVHVGSRGSKKNYQSQADIPIRSPFGIVKASWLPKFSHADAQKMKRLPTPSMMNDYYAASPRIFPHLCSLCNVECSHLKDWIQHQNTSTHIESCRQLRQQYPDWNPEILPSRRNEGNRKENETPRRRSHSPSPRRSRRSSSSHRFRRSRSPMHYMYRPRSRSPRICHRFISRYRSRSRSRSPYRIRNPFRGSPKCFRSVSPERMSRRSVRSSDRKKALEDVVQRSGHGTEFNKQKHLEAADKGHSPAQKPKTSSGTKPSVKPTSATKSDSNLGGHSIRCKSKNLEDDTLSECKQVSDKAVSLQRKLRKEQSLHYGSVLLITELPEDGCTEEDVRKLFQPFGKVNDVLIVPYRKEAYLEMEFKEAITAIMKYIETTPLTIKGKSVKICVPGKKKAQNKEVKKKTLESKKVSASTLKRDADASKAVEIVTSTSAAKTGQAKASVAKVNKSTGKSASSVKSVVTVAVKGNKASIKTAKSGGKKSLEAKKTGNVKNKDSNKPVTIPENSEIKTSIEVKATENCAKEAISDAALEATENEPLNKETEEMCVMLVSNLPNKGYSVEEVYDLAKPFGGLKDILILSSHKKAYIEINRKAAESMVKFYTCFPVLMDGNQLSISMAPENMNIKDEEAIFITLVKENDPEANIDTIYDRFVHLDNLPEDGLQCVLCVGLQFGKVDHHVFISNRNKAILQLDSPESAQSMYSFLKQNPQNIGDHMLTCSLSPKIDLPEVQIEHDPELEKESPGLKNSPIDESEVQTATDSPSVKPNELEEESTPSIQTETLVQQEEPCEEEAEKATCDSDFAVETLELETQGEEVKEEIPLVASASVSIEQFTENAEECALNQQMFNSDLEKKGAEIINPKTALLPSDSVFAEERNLKGILEESPSEAEDFISGITQTMVEAVAEVEKNETVSEILPSTCIVTLVPGIPTGDEKTVDKKNISEKKGNMDEKEEKEFNTKETRMDLQIGTEKAEKNEGRMDAEKVEKMAAMKEKPAENTLFKAYPNKGVGQANKPDETSKTSILAVSDVSSSKPSIKAVIVSSPKAKATVSKTENQKSFPKSVPRDQINAEKKLSAKEFGLLKPTSARSGLAESSSKFKPTQSSLTRGGSGRISALQGKLSKLDYRDITKQSQETEARPSIMKRDDSNNKTLAEQNTKNPKSTTGRSSKSKEEPLFPFNLDEFVTVDEVIEEVNPSQAKQNPLKGKRKETLKNVPFSELNLKKKKGKTSTPRGVEGELSFVTLDEIGEEEDAAAHLAQALVTVDEVIDEEELNMEEMVKNSNSLFTLDELIDQDDCISHSEPKDVTVLSVAEEQDLLKQERLVTVDEIGEVEELPLNESADITFATLNTKGNEGDTVRDSIGFISSQVPEDPSTLVTVDEIQDDSSDLHLVTLDEVTEEDEDSLADFNNLKEELNFVTVDEVGEEEDGDNDLKVELAQSKNDHPTDKKGNRKKRAVDTKKTKLESLSQVGPVNENVMEEDLKTMIERHLTAKTPTKRVRIGKTLPSEKAVVTEPAKGEEAFQMSEVDEESGLKDSEPERKRKKTEDSSSGKSVASDVPEELDFLVPKAGFFCPICSLFYSGEKAMTNHCKSTRHKQNTEKFMAKQRKEKEQNEAEERSSR

SEQ ID NO: 108 - RNA, 102 nt, Homo sapiens
  source 1..102: mol_type = mRNA
  variation 22..102: note = cag repeats may be deleted
cagcagcagcagcagcagcagcagcagcagcagcagcagcagcagcagcagcagcagcagcagcagcagcagcagcagcagcagcagcagcagcagcagcag

SEQ ID NO: 109 - RNA, 75 nt, Homo sapiens
  source 1..75: mol_type = mRNA
cagcagcagcagcagcagcagcagcagcagcagcagcagcagcagcagcagcagcagcagcagcagcagcagcag

SEQ ID NO: 110 - RNA, 15 nt, Homo sapiens
  source 1..15: mol_type = mRNA
cagcagcagcagcag

SEQ ID NO: 111 - RNA, 420 nt, Homo sapiens
  source 1..420: mol_type = mRNA
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 112 - RNA, 60 nt, Homo sapiens
  source 1..60: mol_type = mRNA
cagcagcagcagcagcagcagcagcagcagcagcagcagcagcagcagcagcagcagcag

SEQ ID NO: 113 - RNA, 21 nt, Mus sp.
  source 1..21: mol_type = mRNA
cagcagcagcagcagcagcag

SEQ ID NO: 114 - skipped

(19) United States
(12) Patent Application Publication
(10) Pub. No.: US 2024/0002863 A1
(43) Pub. Date: Jan. 4, 2024

(54) SYNTHETIC SITE-SPECIFIC RNA EDITING ENTITIES

(71) Applicant: ENZERNA BIOSCIENCES, INC., Morrisville, NC (US)

(72) Inventors: Joseph RUIZ, Cary, NC (US); Zefeng WANG, Shanghai (CN)

(21) Appl. No.: 18/338,966

(22) Filed: Jun. 21, 2023

Related U.S. Application Data
(63) Continuation of application No. PCT/US2021/064713, filed on Dec. 21, 2021.
(60) Provisional application No. 63/129,071, filed on Dec. 22, 2020.
(60) Provisional application No. 63/286,843, filed on Dec. 7, 2021.

(51) Int. Cl. C12N 15/117; C12N 15/86; A61P 25/00

(52) U.S. Cl. CPC C12N 15/117; C12N 15/86; A61P 25/00

(57) ABSTRACT

The present disclosure provides compositions comprising synthetic site-specific RNA editing entities engineered to target pathogenic RNA comprising a CAG repeat associated with a CAG repeat disorder. Also disclosed herein are methods of treating the CAG repeat disorders of the present disclosure, such as Huntington's disease with the compositions and pharmaceutical formulations comprising the compositions disclosed herein.

### CROSS REFERENCE

This application is a continuation of International Application No. PCT/US2021/064713, filed on Dec. 21, 2021, which claims the benefit of U.S. Provisional Patent Application No. 63/129,071, filed Dec. 22, 2020, and U.S. Provisional Patent Application No. 63/286,843, filed Dec. 7, 2021, each of which is incorporated herein by reference in its entirety.

### STATEMENT AS TO FEDERALLY SPONSORED RESEARCH

This invention was made with the support of the United States government under Contract number 1R43 NS107101 awarded by the National Institutes of Health. The government has certain rights in the invention.

### REFERENCE TO A SEQUENCE LISTING XML

The instant application contains a Sequence Listing which has been submitted electronically in XML format and is hereby incorporated by reference in its entirety. Said XML file, created on Jun. 14, 2023, is named 55989-702_301_SL.xml and is 122,318 bytes in size.

### SUMMARY

Nucleotide repeat disorders occur when a nucleotide repeat (e.g., CAG) is present in a mutated gene in greater numbers than a non-mutant gene. Currently, there are no curative therapies for nucleotide repeat disorders; it is only possible to provide palliative measures to manage the clinical symptoms.

Many nucleotide repeat disorders are associated with neurodegenerative diseases, such as Huntington's disease (HD), an autosomal dominant disorder that affects ˜1/10,000 individuals. HD is associated with the depletion of neurons and an increased number of glial cells in the region of the brain critical for movement, memory, and decision-making. HD is associated with a CAG repeat that is translated into a polyglutamine repeat within the Huntingtin protein (Htt). Since HD patients can have one normal and one mutated Htt allele with a long CAG repeat, an attractive therapeutic strategy would be to selectively degrade the product of mutated allele.

This disclosure provides synthetic site-specific RNA editing entities that specifically recognize and degrade pathogenic RNAs comprising CAG repeats, as well as methods of treating CAG repeat disorders, such as Huntington's disease. The innovative therapeutic approach disclosed herein targets pathogenic RNA more effectively than existing strategies.

Disclosed herein, in some aspects, are synthetic RNA binding domains comprising an amino acid sequence with at least 90% sequence identity to SEQ ID NO: 6. In some embodiments, the synthetic RNA binding domains comprise at least one mutation at a position corresponding to residues 36-362 of SEQ ID NO: 6. In some embodiments, the synthetic RNA binding domains comprise at least one mutation at a position corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; residues 358 to 362 of SEQ ID NO: 6; or any combination of (a) to (j). In some embodiments, the synthetic RNA binding domains comprise at least one mutation in at least two ranges of residues corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; or residues 358 to 362 of SEQ ID NO: 6. In some embodiments, the synthetic RNA binding domains facilitate cleavage of an RNA comprising a CAG repeat by a synthetic site-specific RNA editing entity, when the synthetic RNA binding domain is present in the synthetic site-specific RNA editing entity and is associated with the RNA. In some embodiments, the CAG repeat comprises a nucleotide sequence that is CAGCAGCAGC (SEQ ID NO: 28), AGCAGCAGCA (SEQ ID NO: 29), GCAGCAGCAG (SEQ ID NO: 30), or any combination thereof. In some embodiments, the synthetic RNA binding domains comprise an amino acid sequence with at least 90% sequence identity to any one of SEQ ID NOs: 7-9. In some embodiments, the synthetic RNA binding domains comprise an amino acid sequence with at least 95%, at least 97%, at least 98%, or at least 99% sequence identity to any one of SEQ ID NOs: 7-9. In some embodiments, the synthetic RNA binding domains comprise an amino acid sequence that is any one of SEQ ID NOs: 7-9. In some embodiments, the RNA comprising the CAG repeat is messenger RNA or pre-messenger RNA.

Disclosed herein, in some aspects, are synthetic RNA binding domains comprising an amino acid sequence with at least 95% sequence identity to SEQ ID NO: 10. In some embodiments, the synthetic RNA binding domains comprise at least one mutation at a position corresponding to residues 36-290 of SEQ ID NO: 10. In some embodiments, the synthetic RNA binding domains comprise at least one mutation at a position corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; residues 286 to 290 of SEQ ID NO: 10; or any combination of (a) to (h). In some embodiments, the synthetic RNA binding domains comprise at least one mutation in at least two ranges of residues corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; or residues 286 to 290 of SEQ ID NO: 10. In some embodiments, the synthetic RNA binding domains facilitate cleavage of an RNA comprising a CAG repeat by a synthetic site-specific RNA editing entity, when the synthetic RNA binding domain is present in the synthetic site-specific RNA editing entity and is associated with the RNA. In some embodiments, the CAG repeat comprises a nucleotide sequence that is CAGCAGCA, AGCAGCAG, GCAGCAGC, or any combination thereof. In some embodiments, the RNA comprising the CAG repeat is messenger RNA or pre-messenger RNA. In some embodiments, the synthetic RNA binding domains comprise an amino acid sequence with at least 92% sequence identity to any one of SEQ ID NOs: 11-13 and 44. In some embodiments, the synthetic RNA binding domains comprise an amino acid sequence with at least 95%, at least 97%, at least 98%, or at least 99% sequence identity to any one of SEQ ID NOs: 11-13 and 44. In some embodiments, the synthetic RNA binding domains comprise an amino acid sequence that is any one of SEQ ID NOs: 11-13 and 44. In some embodiments, the at least one mutation results in synthetic RNA binding domains that have an amino acid sequence comprising SerTyrXxxXxxArg that binds cytosine, wherein Xxx is any amino acid. In some embodiments, the at least one mutation results in synthetic RNA binding domains that have an amino acid sequence comprising (Cys/Ser/Asn)XxxXxxXxxGln that binds adenine, wherein Xxx is any amino acid. In some embodiments, the at least one mutation results in synthetic RNA binding domains that have an amino acid sequence comprising SerXxxXxxXxxGlu that binds guanine, wherein Xxx is any amino acid.

Disclosed herein, in some aspects is a composition comprising an isolated and purified RNA editing entity that comprises the synthetic RNA binding domain of any one of the preceding embodiments.

Disclosed herein, in some aspects are polynucleotide sequences encoding synthetic RNA binding domains of any one of the preceding embodiments.

Disclosed herein, in some aspects are synthetic site-specific RNA editing entities targeting a pathogenic RNA that comprises a CAG repeat, the site-specific RNA editing entities comprising: (i) a synthetic RNA binding domain; and (ii) a cleavage domain; wherein the synthetic RNA binding domain comprises an amino acid sequence comprising (Cys/Ser/Asn)XxxXxxXxxGln that binds to adenine, wherein Xxx is any amino acid.

In some embodiments, the synthetic RNA binding domains comprise an amino acid sequence with at least 90% sequence identity to SEQ ID NO: 6. In some embodiments, the synthetic RNA binding domains comprise at least one mutation at a position corresponding to residues 36-362 of SEQ ID NO: 6. In some embodiments, the synthetic RNA binding domains comprise at least one mutation at a position corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; residues 358 to 362 of SEQ ID NO: 6; or any combination of (a) to (j). In some embodiments, the synthetic RNA binding domains comprise at least one mutation in at least two ranges of residues corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; or residues 358 to 362 of SEQ ID NO: 6. In some embodiments, the synthetic site-specific RNA editing entities facilitate cleavage of the pathogenic RNA that comprises the CAG repeat when the synthetic site-specific RNA editing entity is associated with the pathogenic RNA. In some embodiments, the CAG repeat comprises a nucleotide sequence that is CAGCAGCAGC (SEQ ID NO: 28), AGCAGCAGCA (SEQ ID NO: 29), GCAGCAGCAG (SEQ ID NO: 30), or any combination thereof. In some embodiments, the synthetic RNA binding domains comprise an amino acid sequence with at least 90% sequence identity to any one of SEQ ID NOs: 7-9. In some embodiments, the synthetic site-specific RNA editing entities comprise an amino acid sequence with at least 95%, at least 97%, at least 98%, or at least 99% sequence identity to any one of SEQ ID NOs: 7-9. In some embodiments, the synthetic RNA binding domains comprise an amino acid sequence with at least 99% sequence identity to any one of SEQ ID NOs: 7-9. In some embodiments, the synthetic site-specific RNA editing entities comprise an amino acid sequence that is any one of SEQ ID NOs: 7-9. In some embodiments, the cleavage domain cleaves upstream or downstream of a 10 nucleotide RNA target sequence. In some embodiments, the synthetic RNA binding domains comprise an amino acid sequence with at least 95% sequence identity to SEQ ID NO: 10. In some embodiments, the synthetic RNA binding domains comprise at least one mutation at a position corresponding to residues 36-290 of SEQ ID NO: 10. In some embodiments, the synthetic RNA binding domains comprise at least one mutation at a position corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; residues 286 to 290 of SEQ ID NO: 10; or any combination of (a) to (h). In some embodiments, the synthetic RNA binding domains comprise at least one mutation in at least two ranges of residues corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; or residues 286 to 290 of SEQ ID NO: 10. In some embodiments, the synthetic site-specific RNA editing entities facilitate cleavage of the pathogenic RNA that comprises the CAG repeat when associated with the pathogenic RNA. In some embodiments, the CAG repeat comprises a nucleotide sequence that is CAGCAGCA, AGCAGCAG, GCAGCAGC, or any combination thereof. In some embodiments, pathogenic RNA that comprises the CAG repeat is messenger RNA or pre-messenger RNA. In some embodiments, the synthetic RNA binding domains comprise an engineered human Pumilio 1 domain. In some embodiments, the synthetic RNA binding domains comprise an amino acid sequence with at least 92% sequence identity to any one of SEQ ID NOs: 11-13 and 44. In some embodiments, the synthetic site-specific RNA editing entities comprise an amino acid sequence with at least 95%, at least 97%, at least 98%, or at least 99% sequence identity to any one of SEQ ID NOs: 11-13 and 44. In some embodiments, the synthetic RNA binding domains comprise an amino acid sequence that is any one of SEQ ID NOs: 11-13 and 44. In some embodiments, the synthetic RNA binding domains comprise an amino acid sequence with at least 92% sequence identity to any one of SEQ ID NOs: 35-37. In some embodiments, the synthetic RNA binding domains comprise an amino acid sequence with at least 95%, at least 97%, at least 98%, or at least 99% sequence identity to any one of SEQ ID NOs: 35-37. In some embodiments, the synthetic RNA binding domains comprise an amino acid sequence that is any one of SEQ ID NOs: 35-37. In some embodiments, the synthetic site-specific RNA editing entities have RNA endonuclease activity. In some embodiments, the cleavage domain comprises a PilT N-terminus (PIN) domain or an enzymatically-active variant, derivative, or fragment thereof. In some embodiments, the cleavage domain comprises a PilT N-terminus (PIN) domain of human SMG6. In some embodiments, the at least one mutation results in synthetic RNA binding domains that have an amino acid sequence comprising SerTyrXxxXxxArg that binds cytosine, wherein Xxx is any amino acid. In some embodiments, the at least one mutation results in synthetic RNA binding domains that have an amino acid sequence comprising (Cys/Ser/Asn)XxxXxxXxxGln that binds to adenine, wherein Xxx is any amino acid. In some embodiments, the at least one mutation results in synthetic RNA binding domains that have an amino acid sequence comprising SerXxxXxxXxxGlu that binds to guanine, wherein Xxx is any amino acid. In some embodiments, the cleavage domain comprises an amino acid sequence with at least 90% sequence identity to SEQ ID NO: 41. In some embodiments, the cleavage domain comprises an amino acid sequence with at least 95%, at least 97%, at least 98%, or at least 99% sequence identity to SEQ ID NO: 41. In some embodiments, the cleavage domain comprises an amino acid sequence that is SEQ ID NO: 41. In some embodiments, a C-terminus of the synthetic RNA binding domain is joined to an N-terminus of the cleavage domain. In some embodiments, the synthetic site-specific RNA editing entities further comprise a linker. In some embodiments, a C-terminus of the synthetic RNA binding domain is joined to an N-terminus of a linker and a C-terminus of the linker is joined to an N-terminus of the cleavage domain. In some embodiments, the linker is at least three amino acids in length and at most twenty amino acids in length. In some embodiments, the linker comprises an amino acid sequence from Table 1. In some embodiments, the linker comprises an amino acid sequence that is VDTANGS (SEQ ID NO: 42).

Disclosed herein, in some aspects, are compositions comprising isolated and purified synthetic site-specific RNA editing entities of any one of the preceding embodiments.

Disclosed herein, in some aspects, are polynucleotide sequences encoding the synthetic site-specific RNA editing entity of any one of the preceding embodiments.

Disclosed herein, in some aspects, are vectors comprising the polynucleotide sequence of any one of the preceding embodiments. In some embodiments, the vector is a viral vector. In some embodiments, the vector is an adeno-associated viral vector (AAV), retroviral vector, adenoviral vector, or a lentiviral vector.

Disclosed herein, in some aspects, are pharmaceutical compositions comprising the vector of any one of the preceding embodiments and a pharmaceutically acceptable excipient, carrier, or diluent.

Disclosed herein, in some aspects, are kits comprising the composition of any one of the preceding embodiments, or the pharmaceutical composition of any one of the preceding embodiments.

Disclosed herein, in some aspects, are cells or cell cultures expressing the polynucleotide sequence of any one of the preceding embodiments.

Disclosed herein, in some aspects, are methods of delivering a synthetic site-specific RNA editing entity to a cell, comprising administering to the cell the vector of any one of the preceding embodiments. In some embodiments, the polynucleotide sequence encoding the synthetic site-specific RNA editing entity is integrated into the genome of the cell.

Disclosed herein, in some aspects, are methods of treating a subject in need thereof, comprising administering to the subject the synthetic site-specific RNA editing entity, or an enzymatically-active fragment thereof, the vector, or the pharmaceutical composition of any one of the preceding embodiments.

Disclosed herein, in some aspects, are methods of treating a subject in need thereof, comprising administering to the subject a synthetic site-specific RNA editing entity targeting a pathogenic RNA that comprises a CAG repeat, the site-specific RNA editing entity comprising: (i) a synthetic RNA binding domain; and (ii) a cleavage domain; wherein the synthetic RNA binding domain comprises an amino acid sequence comprising (Cys/Ser/Asn)XxxXxxXxxGln that binds to adenine, wherein Xxx is any amino acid. In some embodiments, the synthetic RNA binding domain comprises an amino acid sequence with at least 90% sequence identity to SEQ ID NO: 6. In some embodiments, the synthetic RNA binding domain comprises an amino acid sequence with at least 95% sequence identity to SEQ ID NO: 10. In some embodiments, the subject has a CAG repeat-associated disorder. In some embodiments, the subject has a CAG repeat-associated neurological disorder. In some embodiments, the subject has a CAG repeat-associated neurodegenerative disorder. In some embodiments, the subject has Huntington's disease (HD), spinocerebellar ataxia (SCA), dentatorubral-pallidoluysian atrophy (DRPLA), or spinal and bulbar muscular atrophy (SBMA). In some embodiments, the subject has Huntington's disease (HD). In some embodiments, the synthetic site-specific RNA editing entities cleave an RNA encoding a pathogenic Huntingtin protein. In some embodiments, administering the synthetic site-specific RNA editing entity or the vector reduces expression of the pathogenic Huntingtin protein by 60% or more relative to expression of the pathogenic Huntingtin protein without the administering. In some embodiments, administering the synthetic site-specific RNA editing entity or the vector reduces expression of a wild-type Huntingtin protein by 40% or less relative to expression of the wild-type Huntingtin protein without the administering. In some embodiments, the subject has spinocerebellar ataxia (SCA) type 1, SCA type 2, SCA type 3, SCA type 6, SCA type 7, or SCA type 17. In some embodiments, the subject has the SCA type 3. In some embodiments, the methods further comprise administering an additional therapeutic agent to the subject. In some embodiments, the additional therapeutic agent is an antipsychotic, a drug to treat chorea, an antidepressant, a mood-stabilizing drug, an anti-inflammatory drug, a neuroprotective drug, or a combination thereof. In some embodiments, the administering comprises parenteral administration. In some embodiments, the administering comprises intracranial injection or intrathecal injection.

Disclosed herein, in some aspects, are a methods of producing a synthetic site-specific RNA editing entity that targets a pathogenic RNA comprising a CAG repeat, the method comprising expressing the synthetic site-specific RNA editing entity of any one of the preceding embodiments in a cell, and harvesting the synthetic site-specific RNA editing entity. In some embodiments, the cell is a bacterium. In some embodiments, the bacterium is Escherichia coli. In some embodiments, the cell is a yeast. In some embodiments, the yeast is Saccharomyces cerevisiae. In some embodiments, the pathogenic RNA is messenger RNA or pre-messenger RNA.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts of this disclosure are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1A shows that ASREs disclosed herein reduce levels of Htt mRNA with 140 copies of a CAG repeat (SEQ ID NO: 111) in mouse embryonic stem cell model.

FIG. 1B shows the effect of ASREs on levels of Htt mRNA with 20 copies of a CAG repeat (SEQ ID NO: 112) in mouse embryonic stem cell model.

FIG. 2 shows that ASREs disclosed herein reduce levels of Htt mRNA with 70 copies of a CAG repeat in primary human fibroblasts from a Huntington's disease (HD) patient.

FIG. 3A shows that an ASRE disclosed herein preferentially reduces levels of ATXN3 mRNA with 71 copies of a CAG repeat in primary human fibroblasts from a spinocerebellar ataxia type 3 (SCA3) patient.

FIG. 3B shows that ASREs disclosed herein preferentially reduce levels of ATXN3 mRNA with 74 copies of a CAG repeat in primary human fibroblasts from a spinocerebellar ataxia type 3 (SCA3) patient.

### DETAILED DESCRIPTION

Nucleotide repeat disorders occur when a nucleotide repeat (e.g., CAG) is present in a mutated gene in greater numbers than a non-mutant gene. Many nucleotide repeat disorders are associated with neurodegenerative diseases, such as Huntington's disease (HD). HD is an autosomal dominant disorder caused by the polyglutamine repeat expansion within the Huntingtin protein (Htt) that affects ˜1/10,000 individuals. HD is associated with the depletion of neurons and an increased number of glial cells in the region of the brain critical for movement, memory, and decision-making. The protein aggregates formed from the polyglutamine-containing peptide are thought to be the main cause of neuronal cell death, although recent results have suggested that the RNA repeat itself may also be directly responsible for neurotoxicity. Currently, there are no curative therapies for nucleotide repeat disorders; it is only possible to provide palliative measures to manage the clinical symptoms.

All Htt proteins have the polyglutamine repeats, but the number of repeats influences the onset, progression, and severity of the disease. Normal individuals can have between 7-34 CAG repeats (SEQ ID NO: 108), while individuals with ≥40 repeats develop HD. Since HD patients can have one normal and one mutated Htt allele with long CAG repeats, an attractive therapeutic strategy would be to selectively degrade the product of mutated allele. Therapeutic strategies such as antisense oligonucleotide (ASO) and RNA interference (RNAi) can be limited, for example, by poor delivery across the blood brain barrier, and passive delivery to target cells in vivo. The use of CRISPR/Cas gene editing technology to correct mutant alleles as a therapeutic can also be limited, as in some cases the final outcome of this gene editing cannot be precisely controlled, potentially leading to unwanted consequences. An approach that targets the pathogenic RNA more effectively than antisense strategies would provide an innovative therapeutic approach. Described herein, in certain embodiments, are synthetic site-specific RNA editing entities recognizing pathogenic RNA comprising CAG repeats as well as methods of treating CAG repeat degenerative disorders, such as Huntington's disease.

### Compositions

Described herein, in certain embodiments, are synthetic site-specific RNA editing entities that target an RNA comprising a CAG repeat (e.g., an mRNA or pre-mRNA). A site-specific RNA editing entity is capable of binding to and cleaving, modifying, editing, or modulating expression of a target RNA (e.g., a pathogenic RNA comprising a CAG repeat). A site-specific RNA editing entity can have nuclease activity (e.g., exonuclease or endonuclease activity). A synthetic site-specific RNA editing entity is engineered as disclosed herein, and is not naturally-occurring.

In some embodiments, the synthetic site-specific RNA editing entity comprises: (i) a synthetic RNA binding domain, (ii) a linker, and (iii) a cleavage domain. In some embodiments the synthetic RNA binding domain binds to an RNA target sequence. In some embodiments, the RNA target sequence is located in an mRNA or pre-mRNA encoding a protein associated with a CAG repeat neurodegenerative disorder. In some embodiments, synthetic site-specific RNA editing entities are also referred to herein as artificial site-specific RNA editing entities (ASREs).

In some embodiments, the synthetic site-specific RNA editing entities described herein comprise a higher affinity for 25 or more CAG repeats than an affinity for 5 or less CAG repeats, for example, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1000-fold higher affinity. In some embodiments, the synthetic site-specific RNA editing entities described herein comprise a higher affinity for 25 CAG repeats (SEQ ID NO: 109) than an affinity for 5 CAG repeats (SEQ ID NO: 110), for example, at least 2-fold, at least 3-fold, at least 4-fold, at least 5-fold, at least 6-fold, at least 7-fold, at least 8-fold, at least 9-fold, at least 10-fold, at least 15-fold, at least 20-fold, at least 25-fold, at least 30-fold, at least 40-fold, at least 50-fold, at least 60-fold, at least 70-fold, at least 80-fold, at least 90-fold, at least 100-fold, at least 200-fold, at least 300-fold, at least 400-fold, at least 500-fold, at least 600-fold, at least 700-fold, at least 800-fold, at least 900-fold, or at least 1000-fold higher affinity. In some embodiments, the synthetic site-specific RNA editing entity comprises an amino acid sequence selected from: SEQ ID NOs: 7-9, SEQ ID NOs: 11-13, SEQ ID NOs: 34-41, SEQ ID NO: 44, and SEQ ID NOs: 46-48.

Synthetic RNA Binding Domain

In some embodiments, the synthetic site-specific RNA editing entity comprises a synthetic RNA binding domain, for example, a variant of an RNA binding domain (e.g., Puf-HUD) that is modified to bind an RNA target sequence that is different than the RNA target sequence bound by an unmodified (e.g., wild type) RNA binding domain. In some embodiments, the synthetic RNA binding domain is a modified Pumilio homology domain (PU-HUD). In some embodiments, the synthetic RNA binding domain is a modified human Pumilio 1 (PUF) domain (e.g., a modified SEQ ID NO: 1 or a fragment thereof).

In some embodiments, the synthetic RNA binding domain contains modular armadillo repeats (ARM repeats), for example, a modular protein that binds RNA in a sequence specific manner, wherein the RNA specificity is changed by modifying the amino acid side chain(s) of the protein. In some embodiments, the synthetic RNA binding domain is a modified version of any PUF protein family member with a Pum-HD domain. Non-limiting examples of a PUF family member include, but are not limited to, FBF in C. elegans, Ds pum in Drosophila and PUF proteins in plants such as Arabidopsis and rice. PUF family members are highly conserved from yeast to human and all members of the family bind to RNA in a sequence specific manner.

In some embodiments, the synthetic RNA binding domain comprises a PUF domain made up of a plurality of 36 mer-repeats. In some embodiments, the PUF domain is made up of eight 36 mer-repeats. In some embodiments, the PUF domain is made up of ten 36 mer-repeats. In some embodiments, in each 36-mer or a subset of 36-mers in the plurality of 36-mer repeats, 33 of the amino acids are conserved and the 34th, 35th, and 36th amino acids are varied to impart specificity for a particular base in an RNA sequence. In some embodiments, in each 36-mer or a subset of 36-mers in the plurality of 36-mer repeats, 34 of the amino acids are conserved and the and 36th amino acids are varied to impart specificity for a particular base in an RNA sequence. In some embodiments, in each 36-mer or a subset of 36-mers in the plurality of 36-mer repeats, 35 of the amino acids are conserved and the 36th amino acids is varied to impart specificity for a particular base in an RNA sequence. In some embodiments, the synthetic RNA binding domain is about 300 (e.g., 310, 309, 308, 307, 306, 305, 304, 303, 302, 301, 300, 299, 298, 297, 296, 295, 294, 293, 292, 291, 290, etc.) amino acids in length. In some embodiments, the synthetic RNA binding domain is about 340 (e.g., 330, 331, 332, 333, 334, 335, 336, 337, 338, 339, 340, 341, 342, 343, 344, 345, 346, 347, 348, 349, or 350) amino acids in length. In some embodiments, the synthetic RNA binding domain is about 412 (e.g., 400, 401, 402, 403, 404, 405, 406, 407, 408, 409, 410, 411, 412, 413, 414, 415, 416, 417, 418, 419, 420, 421, or 422) amino acids in length.

In some embodiments, the variant PUF domain comprises a sequence at least 80%, at least 85%, at least 90%, at least 91%, at least 92%, at least 93%, at least 94%, at least 95%, at least 96%, at least 97%, at least 98%, or at least 99% identical to amino acids 828 to 1176 of SEQ ID NO: 1. In some embodiments, the variant PUF domain comprises a sequence less than 80%, less than 85%, less than 90%, or less than 95% identical to amino acids 828 to 1176 of SEQ ID NO: 1. In some embodiments the variant PUF domain comprises at least one modification relative to amino acids 828 to 1176 of SEQ ID NO: 1. In some embodiments, the modification is a non-naturally occurring amino acid. In some embodiments, the at least one modification results in the variant PUF domain comprising, in any combination SerTyrXxxXxxArg to bind cytosine, (Cys/Ser/Asn)XxxXxxXxxGln to bind adenine, and SerXxxXxxXxxGlu to bind guanine, wherein Xxx is any amino acid.

In some embodiments, the synthetic RNA binding domain comprises a sequence at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 6. In some embodiments, the synthetic RNA binding domain comprises a sequence at least 90% identical to SEQ ID NO: 6. In some embodiments, the synthetic RNA binding domain comprises a sequence at least 91% identical to SEQ ID NO: 6. In some embodiments, the synthetic RNA binding domain comprises a sequence with 90-99%, 90-98%, 90-97%, 90-96%, 90-95%, 91-99%, 91-98%, 91-97%, 91-96%, 91-95%, 92-99%, 92-98%, 92-97%, 92-96%, 92-95%, 93-99%, 93-98%, 93-97%, 93-96%, 93-95%, 94-99%, 94-98%, 94-94%, 94-96%, or 94-95% sequence identity to SEQ ID NO: 6.

In some embodiments, the synthetic RNA binding domain comprises at least one mutation at a position, relative to SEQ ID NO: 6, corresponding to: a) position 36 to 40, position 72 to 76, position 108 to 112, position 144 to 148, position 178 to 182, position 214 to 218, position 250 to 254, position 286 to 290, position 322 to 326, position 358 to 362, or a combination thereof. In some embodiments, the at least one mutation relative to SEQ ID NO: 6 results in the synthetic RNA binding domain comprising a SerTyrXxxXxxArg to bind cytosine, (Cys/Ser/Asn)XxxXxxXxxGln to bind adenine, SerXxxXxxXxxGlu to bind guanine, or a combination thereof. In some embodiments, the synthetic RNA binding domain recognizes (e.g., specifically binds to) a CAG repeat selected from the group consisting of: CAGCAGCAGC (SEQ ID NO: 28), AGCAGCAGCA (SEQ ID NO: 29), and GCAGCAGCAG (SEQ ID NO:30).

In some embodiments, the synthetic RNA binding domain recognizing CAGCAGCAGC (SEQ ID NO: 28) is SEQ ID NO: 7. In some embodiments, the synthetic RNA binding domain recognizing AGCAGCAGCA (SEQ ID NO: 29) is SEQ ID NO: 8. In some embodiments, the synthetic RNA binding domain recognizing GCAGCAGCAG (SEQ ID NO: 30) is SEQ ID NO: 9.

In some embodiments, the synthetic RNA binding domain comprises a sequence at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 10, wherein the synthetic RNA binding domain comprises at least one non-naturally occurring modification. In some embodiments, the at least one non-naturally occurring modification is a non-naturally occurring modification relative to a human Pumilio 1 (PUF) domain (SEQ ID NO:1).

In some embodiments, the synthetic RNA binding domain comprises a sequence at least 95% identical to SEQ ID NO: 10. In some embodiments, the synthetic RNA binding domain comprises a sequence at least 96% identical to SEQ ID NO: 10. In some embodiments, the synthetic RNA binding domain comprises a sequence with 95-99%, 95-98%, 95-97%, 95-96%, 96-99%, 96-98%, or 96-97% sequence identity to SEQ ID NO: 10.

In some embodiments, the synthetic RNA binding domain comprises at least one non-naturally occurring modification compared to SEQ ID NO: 10. In some embodiments, the synthetic RNA binding domain comprises at least one mutation at a position, relative to SEQ ID NO: 10, corresponding to: a) position 36 to 40, position 72 to 76, position 108 to 112, position 144 to 148, position 180 to 184, position 214 to 218, position 250 to 254, position 286 to 290, or a combination thereof. In some embodiments, the at least one mutation relative to SEQ ID NO: 10 results in the synthetic RNA binding domain comprising a SerTyrXxxXxxArg to bind cytosine, (Cys/Ser/Asn)XxxXxxXxxGln to bind adenine, SerXxxXxxXxxGlu to bind guanine, or a combination thereof. In some embodiments, the synthetic RNA binding domain recognizes a CAG repeat selected from the group consisting of: CAGCAGCA, AGCAGCAG, and GCAGCAGC. In some embodiments, the synthetic RNA binding domain recognizing CAGCAGCA is SEQ ID NO: 11. In some embodiments, the synthetic RNA binding domain recognizing AGCAGCAG is SEQ ID NO: 12 or SEQ ID NO: 44. In some embodiments, the synthetic RNA binding domain recognizing GCAGCAGC is SEQ ID NO: 13.

In some embodiments, other RNA binding domains are employed in the synthetic site-specific RNA editing entity, including, for example, RNA binding domains (RBDs) found in splicing proteins, including heteronuclear ribonuclear proteins (HNRNP) and the K homology group of proteins (KH loop proteins), or modified versions thereof.

Further described herein, in certain embodiments, are variant synthetic RNA binding domains as described herein. In some embodiments, the variant synthetic RNA binding domains are isolated and purified. Further described herein, in certain embodiments, are polynucleotide sequences encoding the variant synthetic RNA binding domain described herein.

Linker

In some embodiments, the synthetic site-specific RNA editing entity comprises a linker, for example, a bond, a peptide bond, a linker peptide or a linker sequence. In some embodiments, the linker is a synthetic linker that is heterologous to the amino acids that are joined by the linker. In some embodiments, the linker peptide is about 2, 3, 4, 5, 6, 7, 8, 9, 10, 11, 12, 13, 14, 15, 16, 17, 18, 19, or 20 amino acids in length. In some embodiments, the linker peptide is 7 amino acids in length. In some embodiments, the linker sequence is ideally rich in neutral to polar amino acids that have a slight helical propensity. In some embodiments, the linker peptide forms an alpha helical structure. In some embodiments, proline (Pro) and aromatic amino acids (Phe, Tyr, Trp) are not used in the linker peptide sequence. Thus, in some embodiments, a linker peptide of this disclosure does not comprise a proline, a phenylalanine, a tyrosine, a tryptophan, or a combination thereof. In some embodiments, any suitable linker peptide is used. In some embodiments, the linker peptide is VDTGNGS (SEQ ID NO: 14). In some embodiments, the linker peptide is VDTANGS (SEQ ID NO: 42). In some embodiments, the linker is selected from any one of SEQ ID NOs: 14, 16-27 and 42 and VDT (Table 1).

In some embodiments, the linker peptide contains one or more amino acid insertions, deletions, and/or substitutions relative to a sequence selected from SEQ ID NOs: 14, 16-27 and 42 and VDT, for example, at the N-terminus, the C-terminus, and/or within the sequence. In some embodiments, the linker peptide contains 1, 2, 3, 4, or 5, amino acid insertions relative to a sequence selected from SEQ ID NOs: 14, 16-27 and 42 and VDT. In some embodiments, the linker peptide contains 1, 2, 3, 4, or 5 amino acid deletions relative to a sequence selected from SEQ ID NOs: 14, 16-27 and 42 and VDT. In some embodiments, the linker peptide contains 1, 2, 3, 4, or 5 amino acid substitutions relative to a sequence selected from SEQ ID NOs: 14, 16-27 and 42 and VDT. In some embodiments, the linker peptide comprises an amino acid sequence with at least 50%, at least 60%, at least 70%, at least 80%, at least 90%, or at least 95% sequence identity to a sequence selected from SEQ ID NOs: 14, 16-27 and 42 and VDT. For example, in some embodiments, the cleavage domain is or comprises an amino acid sequence that is SEQ ID NO: 42, which contains one amino acid substitution relative to SEQ ID NO: 14.

TABLE 1
Linker sequences.

| Linker sequence | SEQ ID NO: |
|---|---|
| VDTGNGS | SEQ ID NO: 14 |
| VDT | |
| VDFVGYPRFPAPVEFI | SEQ ID NO: 16 |
| VDMALHARNIA | SEQ ID NO: 17 |
| VDLLALDREVQEL | SEQ ID NO: 18 |
| LLALDREVQE | SEQ ID NO: 19 |
| LLALDREVQ | SEQ ID NO: 20 |
| LLALDREV | SEQ ID NO: 21 |
| VDHIQRGGSP | SEQ ID NO: 22 |
| VDRRMARDGLVH | SEQ ID NO: 23 |
| FVGYPRFPAPVEFI | SEQ ID NO: 24 |
| LLALDREVQEL | SEQ ID NO: 25 |
| MALHARNIA | SEQ ID NO: 26 |
| LGHIQRGGSP | SEQ ID NO: 27 |
| VDTANGS | SEQ ID NO: 42 |

Cleavage Domain

The synthetic RNA editing entity can comprise a cleavage domain. In some embodiments, the cleavage domain is a PilT N-terminus (PIN) domain or an enzymatically-active variant, derivative, or fragment thereof. In some embodiments, the cleavage domain is the PilT N-terminus (PIN) domain of SMG6. In some embodiments, the PIN domain of SMG6 is or comprises residues 1238-1421 of SwissProt Accession No. Q86US8, incorporated herein by reference. In some embodiments, the cleavage domain is or comprises SEQ ID NO: 34. In some embodiments, the cleavage domain is or comprises SEQ ID NO: 41. In some embodiments, any suitable cleavage domain is used in the site-specific RNA editing entities described herein. In some embodiments, the cleavage domain does not exceed 30 KDa. In some embodiments, the cleavage domain has independent activity in trans. In some embodiments, the cleavage domain has an RNAse H/A-like fold at the active site lined by acidic residues (Asp/Glu) or His, which acts via a metal ion (divalent or tetravalent) and cleaves the phosphodiester bond in the nucleic acid backbone.

In some embodiments, the cleavage domain contains one or more amino acid insertions, deletions, and/or substitutions relative to SEQ ID NO: 34, for example, at the N-terminus, the C-terminus, and/or within SEQ ID NO: 34. In some embodiments, the cleavage domain contains 1, 2, 3, 4, 5, 6, 7, 8, 9, or 10 amino acid insertions relative to SEQ ID NO: 34. In some embodiments, the cleavage domain contains 1, 2, 3, 4, 5, 6, 7, 8, 9, or 10 amino acid deletions relative to SEQ ID NO: 34. In some embodiments, the cleavage domain contains 1, 2, 3, 4, 5, 6, 7, 8, 9, or 10 amino acid substitutions relative to SEQ ID NO: 34. In some embodiments, the cleavage domain comprises an amino acid sequence with at least at least 80%, at least 90%, at least 91%, at least 92%, at least 93%, at least 94%, at least 95%, at least 96%, at least 97%, at least 98%, or at least 99% sequence identity to SEQ ID NO: 34. For example, in some embodiments, the cleavage domain is or comprises an amino acid sequence that is SEQ ID NO: 41.

In some embodiments, the cleavage domain contains one or more amino acid insertions, deletions, and/or substitutions relative to SEQ ID NO: 41, for example, at the N-terminus, the C-terminus, and/or within SEQ ID NO: 41. In some embodiments, the cleavage domain contains 1, 2, 3, 4, 5, 6, 7, 8, 9, or 10 amino acid insertions relative to SEQ ID NO: 41. In some embodiments, the cleavage domain contains 1, 2, 3, 4, 5, 6, 7, 8, 9, or 10 amino acid deletions relative to SEQ ID NO: 41. In some embodiments, the cleavage domain contains 1, 2, 3, 4, 5, 6, 7, 8, 9, or 10 amino acid substitutions relative to SEQ ID NO: 41. In some embodiments, the cleavage domain comprises an amino acid sequence with at least 80%, at least 90%, at least 91%, at least 92%, at least 93%, at least 94%, at least 95%, at least 96%, at least 97%, at least 98%, or at least 99% sequence identity to SEQ ID NO: 41.

In some embodiments, the PIN domain of hsMG6 (EST1A; GenBank® Database Accession No. NM 017575, incorporated by reference herein; synonyms include C17orf31, KIAA0732 and SMG-6)) is used in the site-specific RNA editing entity described herein. In some embodiments, the PIN domain has an RnaseH like active site fold and is also very similar in active site architecture to an Archaebacterial PIN domain. In some embodiments, the RNA cleavage domain includes an RNAse A-like fold and/or an RNAse H-like fold.

In some embodiments, the cleavage domain is not a PilT N-terminus (PIN) domain or an enzymatically-active variant, derivative, or fragment thereof. In some embodiments, the cleavage domain is not the PilT N-terminus (PIN) domain of SMG6.

In some embodiments, the cleavage domain comprises, is, or is derived from RNAse 1, RNAse 4, RNAse 6, RNAse 7, RNAse 8, RNAse 2, RNAse 6PL, RNAse L, RNAse T2, RNAse 11, RNAse T2 like, RNAse1 K41R, Rnase1 (K41R, D121E), Rnase1 (K41R, D121E, H119N), Rnase1(H119N), Rnase1(R39D, N67D, N88A, G89D, R91D, H119N), RNAse1(R39D, N67D, N88A, G89D, R91D, H119N, K41R, D121E), Rnase1(R39D, N67D, N88A, G89D, R91D), (Rnase1 (R39D, N67D, N88A, G89D, R91D, H119N, K41R, D121E), NOB1, ENDOV, ENDOG, ENDOD1, hFEN1, ERCC4, NTHL, hSLFN14, hLACTB2, APEX2, ANG, HRSP12, ZC3H12A, APEX1, PDL6, KIAA0391, AGO2, EXOG, ZC3H12D, ERN2, PELO, YBEY, CPSF4L, hCG 2002731, hCG 2002731, ERCC1, RAC1, RAA1, RAB1, DNA2, FLJ35220, F1113173, TENM1, TENM2, RNAseK, TALEN, or ZNF638.

In some embodiments, the cleavage domain comprises, consists essentially of, or consists of an enzymatically-active variant, derivative, or fragment thereof of RNAse 1, RNAse 4, RNAse 6, RNAse 7, RNAse 8, RNAse 2, RNAse 6PL, RNAse L, RNAse T2, RNAse 11, RNAse T2 like, RNAse1 K41R, Rnase1 (K41R, D121E), Rnase1 (K41R, D121E, H119N), Rnase1(H119N), Rnase1(R39D, N67D, N88A, G89D, R91D, H119N), RNAse1(R39D, N67D, N88A, G89D, R91D, H119N, K41R, D121E), Rnase1(R39D, N67D, N88A, G89D, R91D), (Rnase1 (R39D, N67D, N88A, G89D, R91D, H119N, K41R, D121E), NOB1, ENDOV, ENDOG, ENDOD1, hFEN1, ERCC4, NTHL, hSLFN14, hLACTB2, APEX2, ANG, HRSP12, ZC3H12A, APEX1, PDL6, KIAA0391, AGO2, EXOG, ZC3H12D, ERN2, PELO, YBEY, CPSF4L, hCG 2002731, hCG 2002731, ERCC1, RAC1, RAA1, RAB1, DNA2, FLJ35220, F1113173, TENM1, TENM2, RNAseK, TALEN, or ZNF638. In some embodiments, the cleavage domain comprises, consists essentially of, or consists of an enzymatically-active variant, derivative, or fragment thereof of any one of SEQ ID NOs: 49-107.

In some embodiments, the cleavage domain comprises an amino acid sequence with at least at least 80%, at least 90%, at least 91%, at least 92%, at least 93%, at least 94%, at least 95%, at least 96%, at least 97%, at least 98%, or at least 99% sequence identity to any one of SEQ ID NOs: 49-107. For example, in some embodiments, the cleavage domain comprises, consists essentially of, or consists of any one of SEQ ID NOs: 49-107.

In some embodiments, the cleavage domain contains one or more amino acid insertions, deletions, and/or substitutions relative to any one of SEQ ID NOs: 49-107, for example, at the N-terminus, the C-terminus, and/or within any one of SEQ ID NOs: 49-107. In some embodiments, the cleavage domain contains 1, 2, 3, 4, 5, 6, 7, 8, 9, or 10 amino acid insertions relative to any one of SEQ ID NOs: 49-107. In some embodiments, the cleavage domain contains 1, 2, 3, 4, 5, 6, 7, 8, 9, or 10 amino acid deletions relative to any one of SEQ ID NOs: 49-107. In some embodiments, the cleavage domain contains 1, 2, 3, 4, 5, 6, 7, 8, 9, or 10 amino acid substitutions relative to any one of SEQ ID NOs: 49-107.

In some embodiments, the cleavage domain comprises, is, or is derived from a Zinc Finger CCCH-Type polypeptide, for example, ZC3H12A or ZC3H12D. In some embodiments, the cleavage domain comprises, is, or is derived from a Zinc Finger CCCH-Type Containing 12A polypeptide (e.g., human ZC3H112A). In some embodiments, the cleavage domain comprises or is the E17 RNA endonuclease derived from human ZC3H112A.

In some embodiments, the cleavage domain comprises an amino acid sequence with at least at least 80%, at least 90%, at least 91%, at least 92%, at least 93%, at least 94%, at least 95%, at least 96%, at least 97%, at least 98%, or at least 99% sequence identity to SEQ ID NO: 81. For example, in some embodiments, the cleavage domain comprises, consists essentially of, or consists of SEQ ID NO: 81. In some embodiments, the cleavage domain contains one or more amino acid insertions, deletions, and/or substitutions relative to SEQ ID NO: 81, for example, at the N-terminus, the C-terminus, and/or within SEQ ID NO: 81. In some embodiments, the cleavage domain contains 1, 2, 3, 4, 5, 6, 7, 8, 9, or 10 amino acid insertions relative to SEQ ID NO: 81. In some embodiments, the cleavage domain contains 1, 2, 3, 4, 5, 6, 7, 8, 9, or 10 amino acid deletions relative to SEQ ID NO: 81. In some embodiments, the cleavage domain contains 1, 2, 3, 4, 5, 6, 7, 8, 9, or 10 amino acid substitutions relative to SEQ ID NO: 81.

In some embodiments, the synthetic RNA binding domain is at the amino terminus of the RNA editing entity and the cleavage domain is at the carboxy terminus of the RNA editing entity. In some embodiments, in this orientation sequence specific cleavage is achieved. In some embodiments, the synthetic RNA binding domain is on the amino terminal side of the cleavage domain, but is not necessarily at the amino terminus, e.g., additional amino acids can be present at the amino terminus. In some embodiments, the cleavage domain is on the carboxy terminal side of the RNA binding domain, but is not necessarily at the carboxy terminus, e.g., additional amino acids can be present at the carboxy terminus.

In some embodiments, the cleavage domain is at the amino terminus of the RNA editing entity and the synthetic RNA binding domain is at the carboxy terminus of the RNA editing entity. In some embodiments, in this orientation, nonspecific cleavage of RNA is achieved. In some embodiments, the synthetic RNA binding domain is on the carboxy terminal side of the cleavage domain, but is not necessarily at the carboxy terminus, e.g., additional amino acids can be present at the carboxy terminus. In some embodiments, the cleavage domain is on the amino terminal side of the RNA binding domain, but is not necessarily at the amino terminus, e.g., additional amino acids can be present at the amino terminus.

In some embodiments, the RNA cleavage domain is about 100 to about 200 amino acids in length. In some embodiments, the RNA cleavage domain is about 100 to about 150 amino acids in length. In some embodiments, the RNA cleavage domain is about 150 to about 200 amino acids in length. In some embodiments, the RNA cleavage domain is about 120 amino acids in length. In some embodiments, the RNA cleavage domain is about 180 amino acids in length. In some embodiments, the RNA cleavage domain is about 181 amino acids in length.

In some embodiments, the synthetic site-specific RNA editing entity is designed to bind to a specific RNA sequence, referred to herein as an RNA target sequence, of about 8, 9, 10, 11, 12, 13, 14, 15, or 16 contiguous RNA bases to position the cleavage domain to cut the target RNA at a specific site. In some embodiments, the RNA target sequence is present in an mRNA or pre-mRNA. In some embodiments, the RNA target sequence is a sequence of 10 contiguous RNA bases to position the cleavage domain to cut the target RNA at a specific site. In some embodiments, the RNA is cut between any of the contiguous RNA target sequence bases as well as at any site upstream and/or downstream of the RNA target sequence. In some embodiments, the target RNA is cut about 1, 2, 3, 4, 5, 6, 7, 8, 9, 10 or more nucleotides upstream or downstream of the RNA target sequence. In some embodiments, the fifth nucleotide of the 8-nt sequence is a U or C, while the other 7 nucleotides vary.

RNA Target Sequence and Target RNA

A synthetic RNA binding domain of the disclosure can recognize and specifically bind to an RNA target sequence. A synthetic site-specific RNA editing entity of the disclosure can recognize and specifically bind to an RNA target sequence via a synthetic RNA binding domain. The RNA target sequence can be present in a target RNA. A target RNA comprises an RNA target sequence and can be, for example, a pathogenic RNA, an RNA comprising a CAG repeat, a messenger RNA, a pre-messenger RNA, or any combination thereof.

In some embodiments, the target RNA is an RNA comprising a CAG repeat (e.g., an mRNA or pre-mRNA). In some embodiments, the target RNA is also referred to herein as a pathogenic RNA or pathogenic mRNA. In some embodiments, the pathogenic RNA or pathogenic mRNA encodes a pathogenic protein. In some embodiments, the pathogenic protein is associated with a CAG repeat disorder, also referred to as a polyglutamine-repeat disorder. In some embodiments, the CAG repeat disorder is a CAG repeat neurodegenerative disorder. In some embodiments, the CAG repeat neurodegenerative disorder is Huntington's disease (HD), spinocerebellar ataxia (SCA), dentatorubral-pallidoluysian atrophy (DRPLA), or spinal and bulbar muscular atrophy (SBMA).

In some embodiments, the RNA target sequence is an 8mer, 9 mer, 10 mer, 11 mer, 12mer, 13mer, 14mer, 15mer, or 16mer. In some embodiments, the RNA target sequence is a 10 mer. In some embodiments, the RNA target sequence is or comprises CAGCAGCAGC (SEQ ID NO: 28), AGCAGCAGCA (SEQ ID NO: 29), or GCAGCAGCAG (SEQ ID NO: 30). In some embodiments, the ability to introduce modifications into the amino acid sequence of the RNA binding domain to alter its specificity for an RNA target sequence is based on the known interactions of bases with the different amino acid side chains of the RNA binding domain (e.g., Puf protein). In some embodiments, the target RNA is an mRNA. In some embodiments, the target RNA is a pre-mRNA.

Huntington's Disease

In some embodiments, the CAG repeat neurodegenerative disorder is Huntington's disease (HD). In some embodiments, the Huntington's disease is caused by a pathogenic Huntingtin protein (Htt). In some embodiments, the target RNA is an RNA encoding the pathogenic Huntingtin protein (Htt). In some embodiments, a target RNA encoding the pathogenic Huntingtin protein comprises a higher number of CAG repeats than an RNA encoding a non-pathogenic Huntingtin protein. In some embodiments, the RNA encoding the pathogenic Htt comprises 40 or more CAG repeats. In some embodiments, the RNA encoding the pathogenic Htt comprises 35 or more, 37 or more, 40 or more, 45 or more, 50 or more, or 60 or more CAG repeats.

In some embodiments, the RNA encoding the non-pathogenic Htt comprises less than 40 CAG repeats. In some embodiments, the RNA encoding the non-pathogenic Htt comprises less than 35 CAG repeats. In some embodiments, the RNA encoding the non-pathogenic Htt comprises less than 34 CAG repeats. In some embodiments, the RNA encoding the non-pathogenic Htt comprises less than 30 CAG repeats. In some embodiments, an RNA encoding a non-pathogenic Htt comprises between 7 and 34 CAG repeats (SEQ ID NO: 108).

### Spinocerebellar Ataxia

In some embodiments, the CAG repeat neurodegenerative disorder is spinocerebellar ataxia (SCA). In some embodiments, the SCA is SCA1, SCA2, SCA3, SCA6, SCAT, or SCA17.

In some embodiments, the spinocerebellar ataxia is spinocerebellar ataxia type 1 (SCA1). In some embodiments, the SCA1 is caused by a pathogenic ataxin-1 (ATXN1) protein. In some embodiments, an RNA encoding the pathogenic ATXN1 comprises a higher number of CAG repeats than an RNA encoding a non-pathogenic ATXN1. In some embodiments, the target RNA is an RNA encoding the pathogenic ataxin-1 (ATXN1) protein. In some embodiments, the RNA encoding a pathogenic ataxin-1 protein comprises 40 or more CAG repeats. In some embodiments, the RNA encoding a non-pathogenic ataxin-1 protein comprises less than 40 CAG repeats.

In some embodiments, the spinocerebellar ataxia is spinocerebellar ataxia type 2 (SCA2). In some embodiments, SCA2 is caused by a pathogenic ataxin-2 (ATXN2) protein. In some embodiments, an RNA encoding the pathogenic ATXN2 comprises a higher number of CAG repeats than an RNA encoding a non-pathogenic ATXN2. In some embodiments, the target RNA is an RNA sequence encoding the pathogenic ATXN2. In some embodiments, the RNA encoding a pathogenic ataxin-2 protein comprises 32 or more CAG repeats. In some embodiments, the RNA encoding a pathogenic ataxin-2 protein comprises 45 or more CAG repeats. In some embodiments, the RNA encoding a non-pathogenic ataxin-2 protein comprises less than 45 CAG repeats. In some embodiments, the RNA encoding a non-pathogenic ataxin-2 protein comprises less than 32 CAG repeats.

In some embodiments, the spinocerebellar ataxia is spinocerebellar ataxia type 3 (SCA3), also known as Machado-Joseph disease. In some embodiments, SCA3 is caused by a pathogenic ataxin-3 (ATXN3) protein. In some embodiments, an RNA encoding the pathogenic ATXN3 comprises a higher number of CAG repeats than an RNA encoding a non-pathogenic ATXN3. In some embodiments, the target RNA is an RNA encoding the pathogenic ATXN3. In some embodiments, the RNA encoding a pathogenic ataxin-3 protein comprises 52 or more CAG repeats. In some embodiments, the RNA encoding a pathogenic ataxin-3 protein comprises 40 or more CAG repeats. In some embodiments, the RNA encoding a pathogenic ataxin-3 protein comprises 60 or more CAG repeats. In some embodiments, the RNA encoding a pathogenic ataxin-3 protein comprises 70 or more CAG repeats. In some embodiments, the RNA encoding a non-pathogenic ataxin-3 protein comprises less than 52 CAG repeats. In some embodiments, the RNA encoding a non-pathogenic ataxin-3 protein comprises less than 44 CAG repeats. In some embodiments, the RNA encoding a non-pathogenic ataxin-3 protein comprises less than 30 CAG repeats.

In some embodiments, the spinocerebellar ataxia is spinocerebellar ataxia type 6 (SCA6). In some embodiments, SCA6 is caused by a pathogenic calcium voltage-gated channel subunit alpha1 A (CACNA1A). In some embodiments, an RNA encoding the pathogenic CACNA1A comprises a higher number of CAG repeats than an RNA encoding a non-pathogenic CACNA1A. In some embodiments, the target RNA is an RNA encoding the pathogenic CACNA1A. In some embodiments, the RNA encoding a pathogenic CACNA1A comprises 20 or more CAG repeats. In some embodiments, the RNA encoding a non-pathogenic CACNA1A comprises 20 or less CAG repeats. In some embodiments, the RNA encoding a non-pathogenic CACNA1A comprises 18 or less CAG repeats.

In some embodiments, the spinocerebellar ataxia is spinocerebellar ataxia type 7 (SCA7). In some embodiments, SCA7 is caused by a pathogenic ataxin-7 (ATXN7) protein. In some embodiments, an RNA encoding the pathogenic ATXN7 comprises a higher number of CAG repeats than an RNA encoding a non-pathogenic ATXN7. In some embodiments, the target RNA is an RNA sequence encoding the pathogenic ATXN7. In some embodiments, the RNA encoding a pathogenic ataxin-7 protein comprises 37 or more CAG repeats. In some embodiments, the RNA encoding a non-pathogenic ataxin-7 protein comprises less than 37 CAG repeats. In some embodiments, the RNA encoding a non-pathogenic ataxin-7 protein comprises less than 37 CAG repeats. In some embodiments, the RNA encoding a non-pathogenic ataxin-7 protein comprises 17 or less CAG repeats.

In some embodiments, the spinocerebellar ataxia is spinocerebellar ataxia type 17 (SCA17). In some embodiments, SCA17 is caused by a pathogenic TATA-binding protein (TBP). In some embodiments, an RNA encoding the pathogenic TBP comprises a higher number of CAG repeats than an RNA encoding a non-pathogenic TBP. In some embodiments, the target RNA is an RNA encoding the pathogenic TBP. In some embodiments, the RNA encoding a pathogenic TBP comprises 43 or more CAG repeats. In some embodiments, the RNA encoding a non-pathogenic TBP comprises 42 or less CAG repeats.

### Dentatorubral-Pallidoluysian Atrophy

In some embodiments, the CAG repeat neurodegenerative disorder is dentatorubral-pallidoluysian atrophy (DRPLA). In some embodiments, DRPLA is caused by a pathogenic atrophin 1 (ATN1). In some embodiments, an RNA encoding the pathogenic ATN1 comprises a higher number of CAG repeats than an RNA encoding a non-pathogenic ATN1. In some embodiments, the target RNA is an RNA encoding the pathogenic ATN1. In some embodiments, the RNA encoding a pathogenic ATN1 comprises 48 or more CAG repeats. In some embodiments, the RNA encoding a non-pathogenic ATN1 comprises less than 48 CAG repeats. In some embodiments, the RNA encoding a non-pathogenic ATN1 comprises 35 or less CAG repeats.

### Spinal and Bulbar Muscular Atrophy

In some embodiments, the CAG repeat neurodegenerative disorder is spinal and bulbar muscular atrophy (SBMA), also referred to as Kennedy's disease. In some embodiments, SBMA is caused by a pathogenic androgen receptor (AR). In some embodiments, an RNA encoding the pathogenic AR comprises a higher number of CAG repeats than an RNA encoding a non-pathogenic AR. In some embodiments, the target RNA is an RNA encoding the pathogenic AR. In some embodiments, the pathogenic RNA encoding a pathogenic androgen receptor protein comprises 38 or more CAG repeats. In some embodiments, a non-pathogenic RNA encoding a non-pathogenic androgen receptor protein comprises 37 or less CAG repeats. In some embodiments, a non-pathogenic RNA encoding a non-pathogenic androgen receptor protein comprises 36 or less CAG repeats.

### Pharmaceutical Compositions and Kits

Described herein, in certain embodiments, are compositions comprising the site-specific RNA editing entities, or any portion(s) thereof, described herein. In some embodiments, the compositions are pharmaceutical compositions and further comprise a pharmaceutically acceptable carrier.

The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers include, but are not limited to, phosphate buffered saline solutions, water, emulsions, such as oil/water emulsions, various types of wetting agents, and sterile solutions. In some embodiments, a carrier is formulated by conventional methods and administered to the subject at a suitable dose. In some embodiments, the compositions are sterile. In some embodiments, the composition contains adjuvants such as preservative, emulsifying agents and dispersing agents. In some embodiments, the composition comprises an antibacterial agent or antifungal agent.

In some embodiments, compositions comprising the site-specific RNA editing entity or a polynucleotide encoding the site-specific RNA editing entity is formulated for delivery to a cell. In some embodiments, the cell is a mammalian cell. In some embodiments, the cell is a human cell. In some embodiments, the delivery is performed in vivo. In some embodiments, the delivery is performed in vitro. In some embodiments, the site-specific RNA editing entity or a polynucleotide encoding the site-specific RNA editing entity is delivered to the cell via a vector.

Described herein, in certain embodiments, are vectors comprising the polynucleotide encoding the site-specific RNA editing entities described herein. In some embodiments, the site-specific RNA editing entity is formulated within a vector for delivery to a cell or to the subject. In some embodiments, the vector is a viral vector or a non-viral vector. In some embodiments, the cell is a mammalian cell. In some embodiments, the mammalian cell is a human cell. In some embodiments, the subject is a mammal. In some embodiments, the subject is a human.

In some embodiments, the vector is a viral vector. In some embodiments, the viral vector is a retroviral vector, an adenoviral vector (e.g., Adenovirus type 5), an adeno associated virus (AAV) vector, an alphavirus vector, a vaccinia virus vector, a herpes simplex virus (HSV) vector, a lentivirus vector, or a retrovirus vector. In some embodiments, the viral vector is a replication-competent viral vector or a replication-incompetent viral vector. In some embodiments, the viral vector comprises an RDG modification to target integrin receptors.

In some embodiments, the viral vector is a non-enveloped virus. In some embodiments, the viral vector is a single-stranded DNA virus. In some embodiments, the viral vector is an adeno associated virus (AAV) vector. In some embodiments, the AAV contains rep and cap genes, wherein the rep gene is required for viral replication and the cap gene is required for the synthesis of capsid proteins. In some embodiments, the adeno associated viral vector is AAV1, AAV2, AAV3, AAV4, AAV5, AAV6, AAV7, AAV8, AAV9, AAV10, AAV11, or AAV12 serotype, scAAV (self-complementary AAV), chimeric, or hybrid AAV, or any combination, derivative, or variant thereof.

In some embodiments, the AAV is modified, for example, to alter tropism, immunogenicity, replication or packaging efficiency, cargo capacity, or any combination thereof. In some embodiments, the AAV is a hybrid AAV, for example, comprising a capsid protein of one AAV serotype and genomic material from another AAV serotype. In some embodiments, the AAV comprises genetic and/or protein sequences derived from two or more AAV serotypes, and can include mutations made to the genetic sequences of those two or more AAV serotypes. In some embodiments, an AAV comprises a chimeric AAV capsid, for example, a capsid protein with one or more regions of amino acids derived from two or more AAV serotypes.

In some embodiments, an AAV genome carries two viral genes: rep and cap. In some embodiments, the virus can utilize two promoters and alternative splicing to generate four proteins necessary for replication (Rep78, Rep68, Rep52, and Rep40), while a third promoter generates the transcript for three structural viral capsid proteins 1, 2, and 3 (VP1, VP2, and VP3), through a combination of alternate splicing and alternate translation start codons.

In some embodiments, vectors contain, at a minimum, sequences encoding an AAV Rep protein or a fragment thereof. In some embodiments, vectors contain AAV Cap, Rep, and AAP proteins. In vectors in which AAV rep and cap (including AAP) sequences are provided, the AAV rep and AAV cap sequences can originate from an AAV of the same Glade. Alternatively, provided herein can be vectors in which a rep sequences are from an AAV source which differs from that which is providing the cap sequences.

In some embodiments, each end of the AAV single-stranded DNA genome contains an inverted terminal repeat (ITR). In some embodiments, said ITRs are the only cis-acting element required for genome replication and packaging. An ITR can be from any AAV serotype. For example, an ITR can be from the following AAV serotypes, AAV1, AAV2, AAV3, AAV4, AAV5, AAV6, AAV7, AAV8, AAV9, AAV10, AAV11, or AAV12.

Suitable host cells that can be used to produce AAV virions or viral particles include yeast cells, insect cells, microorganisms, and mammalian cells.

Non-limiting examples of AAV vectors are provided in, for example, Hudry, et al. “Therapeutic AAV gene transfer to the nervous system: a clinical reality.” Neuron 101.5 (2019): 839-862; Weinmann et al. “Next-generation AAV vectors for clinical use: an ever-accelerating race.” Virus Genes 53.5 (2017): 707-713; Hardcastle et al. “AAV gene delivery to the spinal cord: serotypes, methods, candidate diseases, and clinical trials.” Expert opinion on biological therapy 18.3 (2018): 293-307, and Patent Application Nos. WO2017100671A1, WO2016130591A2, WO2019173538A1, WO2019076856A1, WO2020142236A1, WO2019169004A1, WO2018226785A1, WO2017049252A1, each of which is incorporated herein by reference for such disclosure.

In some embodiments, the non-viral vector is a plasmid, a naked nucleic acid, or nucleic acid complexed with a delivery vehicle. In some embodiments, the plasmid is complexed with a delivery vehicle. In some embodiments, the delivery vehicle is a lipid. In some embodiments, the lipid is a liposome. In some embodiments, the delivery vehicle is a polyplex.

In some embodiments, the lipid is a cationic lipid, an anionic lipid, or neutral lipid. In some embodiments, the lipid is a liposome, a small unilamellar vesicle (SUV), a lipidic envelope, a lipidoid, or a lipid nanoparticle (LNP). In some embodiments, the lipid is mixed with the nucleic acid to form a lipoplex (a nucleic acid-liposome complex). In some embodiments, the lipid is conjugated to the nucleic acid. In some embodiments, the liposome further comprises an additional moiety. In some embodiments, the moiety is a polymer, a lipid, a peptide, a magnetic nanoparticle (MNP), an additional compound, or a combination thereof. In some embodiments, the polymer, lipid, or magnetic nanoparticle is attached to the liposome or integrated into the liposomal membrane. In some embodiments, the polymer is polyethylene glycol (PEG). In some embodiments, the polymer is polysorbate-80-stabilized poly (D,L-lactide-co-glycolate) (PLGA), N-[2-hydroxypropyl] methacrylamide (HPMA), poly(2-(dimethylamino)ethyl methacrylate) (pDMAEMA), or arginine-grafted bioreducible polymers (ABPs). In some embodiments, the peptide is a cell-penetrating peptide, a cell adhesion peptide, or a peptide which binds to a receptor on a cell. In some embodiments, the moiety improves the ability of the liposome to cross the blood brain barrier.

In some embodiments, the polyplex is a polymer-nucleic acid complex. In some embodiments, the polymer in the polyplex is a polyethylenimine (PEI) or a polyamine. In some embodiments, the nucleic acid in the polyplex is a nucleic acid encoding the site-specific RNA editing entity. In some embodiments, the polyplex is further encapsulated in a liposome. In some embodiments, the viral vector is further encapsulated in a liposome.

In some embodiments, the compositions comprising the site-specific RNA editing entity or polynucleotide encoding the site-specific RNA editing entity are formulated for parenteral injection (e.g., via injection or infusion, including intraarterial, intracardiac, intracranial, intradermal, intraduodenal, intramedullary, intramuscular, intraosseous, intraperitoneal, intrathecal, stereotaxic, intravascular, intravenous, intravitreal, epidural and/or subcutaneous). In some embodiments, the compositions described herein are formulated for stereotaxic injection or infusion. In some embodiments, the compositions described herein are formulated for intracranial injection. In some embodiments, the compositions further comprise an agent to increase permeability of the blood brain barrier. In some embodiments, the agent to increase permeability of the blood brain barrier is a vasoactive peptide, a pharmacological agent, or an osmotic agent. In some embodiments, the vasoactive peptide is bradykinin or a bradykinin analog. In some embodiments, the bradykinin analog is RMP-7. In some embodiments, the pharmacological agent to increase permeability of the blood brain barrier is an adenosine agonist or a P-glycoprotein antagonist. In some embodiments, the osmotic agent is mannitol or arabinose. In some embodiments, the agent to increase permeability of the blood brain barrier is injected before, simultaneously with, or after the composition comprising the site-specific RNA editing entity.

Described herein, in certain embodiments, are kits comprising: the synthetic site-specific RNA editing entities described herein or pharmaceutical compositions thereof. In some embodiments, the kit comprises a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In some embodiments, the container is formed from a variety of materials such as glass or plastic.

In some embodiments, the kit includes an identifying description, a label, or a package insert. In some embodiments, the label or package insert lists contents of kit or the pharmaceutical composition, instructions relating to its use in the methods described herein, or a combination thereof. In some embodiments, the label is on or associated with the container. In some embodiments, the label is on a container when letters, numbers, or other characters forming the label are attached, molded or etched into the container itself. In some embodiments, the label is associated with a container when it is present within a receptacle or carrier that also holds the container, e.g., as a package insert. In some instances, the label is used to indicate that the contents are to be used for a specific therapeutic application.

### Methods of Treatment

Described herein, in certain embodiments, are methods of treating a subject with a CAG repeat disorder (e.g., a neurodegenerative disorder) comprising administering to the subject a synthetic site-specific RNA editing entity described herein. In some embodiments, the CAG repeat neurodegenerative disorder is Huntington's disease (HD), spinocerebellar ataxia (SCA), dentatorubral-pallidoluysian atrophy (DRPLA), or spinal and bulbar muscular atrophy (SBMA). In some embodiments, the SCA is SCA1, SCA2, SCA3, SCA6, SCA7, or SCA17. In some embodiments, the CAG repeat neurodegenerative disorder is Huntington's disease (HD).

Disclosed herein, in some embodiments are methods of treating a CAG repeat disorder in a subject, the methods comprising administering to the subject a synthetic site-specific RNA editing entity comprising an RNA binding protein specific to a CAG repeat in a pathogenic RNA or an RNA that encodes a pathogenic protein. In some embodiments, the pathogenic RNA or protein is reduced in the subject following administration by greater than or equal to about 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 99%, or 100%, as compared with expression of the pathogenic RNA or protein in a reference subject having the CAG repeat disorder, or the subject prior to the administration.

In some embodiments, the CAG repeat disorder comprises Huntington's disease (HD), spinocerebellar ataxia (SCA), dentatorubral-pallidoluysian atrophy (DRPLA), or spinal and bulbar muscular atrophy (SBMA). In some embodiments, the SCA comprises or the SCA is SCA1, SCA2, SCA3 (Machado-Joseph disease), SCA6, SCA7, or SCA17. In some embodiments, the synthetic site-specific RNA editing entity comprises an RNA binding domain that specifically binds to a target RNA sequence in a subject that has, or is suspected of having, the CAG repeat disorder. In some embodiments, the RNA target sequence is an 8mer, 9 mer, 10 mer, 11 mer, 12mer, 13mer, 14mer, 15mer, or 16mer. In some embodiments, the RNA target sequence is a 8mer. In some embodiments, the RNA target sequence is a 10 mer. In some embodiments, the RNA target sequence is or comprises CAGCAGCAGC (SEQ ID NO: 28), AGCAGCAGCA (SEQ ID NO: 29), or GCAGCAGCAG (SEQ ID NO: 30), CAGCAGCA, AGCAGCAG, or GCAGCAGC. In some embodiments, the subject has, or is suspected of having, a CAG repeat (such as a CAG repeat expansion) in an RNA that encodes a pathogenic protein that comprises a pathogenic Huntingtin (Htt) protein, a pathogenic ataxin-1 (ATXN1) protein, a pathogenic ataxin-2 (ATXN2) protein, a pathogenic ataxin-3 (ATXN3) protein, a pathogenic calcium voltage-gated channel subunit alpha1 A (CACNA1A), a pathogenic ataxin-7 (ATXN7) protein, a pathogenic TATA-binding protein (TBP), a pathogenic atrophin 1 (ATN1), a pathogenic androgen receptor (AR), or any combination thereof.

In some embodiments, administering a synthetic site-specific RNA editing entity results in cleavage of the target RNA in the subject. In some embodiments, the synthetic site-specific RNA editing entity is administered to the subject in a concentration sufficient to cleave at least 20%, at least 30%, at least 40%, at least 50%, at least 60%, at least 70%, at least 80%, at least 90%, at least 95%, at least 99%, or at least 100% of the target RNA.

In some embodiments, administering the compositions described herein, results in an improvement of a symptom associated with the CAG repeat neurodegenerative disorder. In some embodiments, improvement of the symptom is determined from a patient reported outcome measurement. In some embodiments, the patient reported outcome measurement is a Patient-Reported Outcomes Measurement information System (PROMIS) or Quality of Life in Neurological Disorders (Neuro-QoL) assessment. In some embodiments, the symptom is chorea, dystonia, dysarthria, dysphagia, slow or abnormal eye movement, abnormal gait, abnormal posture or balance, or any combination thereof. In some embodiments, an improvement of the symptoms comprises an elimination of the symptoms.

In some embodiments, the method further comprises administering additional therapeutic agent. In some embodiments, the additional therapeutic agent is administered to treat a symptom of the CAG repeat neurodegenerative disorder. In some embodiments, the additional therapeutic agent is an antipsychotic, a drug to treat chorea, an antidepressant, a mood-stabilizing drug, an anti-inflammatory drug, a neuroprotective drug, or any combination thereof. In some embodiments, the additional therapeutic agent is a cannabinoid. In some embodiments, the antipsychotic is haloperidol, chlorpromazine, risperidone, quetiapine, benzodiazepine, or olanzapine. In some embodiments, the drug to treat chorea is amatadine, levetiracetam, clonazepam, tetrabenazine, or deutetrabenazine. In some embodiments, the antidepressant is citalopram, escitalopram, fluoxetine, sertraline, or aripiprazole. In some embodiments, the mood-stabilizing drug is valproate, carbamazepine, or lamotrigine. In some embodiments, the anti-inflammatory drug is laquinimod. In some embodiments, the neuroprotective drug is prodopidine or a phosphodiesterase inhibitor. In some embodiments, the additional therapeutic agent is injected before, simultaneously with, or after the composition comprising the site-specific RNA editing entity.

Described herein, in certain embodiments, are methods of delivering a synthetic site-specific RNA editing entity to a subject that obviates a need for lifelong administration. In some embodiments, the method comprises administering to the subject a vector comprising a polynucleotide sequence encoding the synthetic site-specific RNA editing entity described herein. In some embodiments, the method comprises stably integrating the polynucleotide sequence into a genome of the subject, thereby obviating a need for lifelong administration of the vector to the subject. In some embodiments, the polynucleotide sequence encoding the synthetic site-specific RNA editing entity is integrated into a safe harbor locus.

A variety of enzymes can catalyze insertion of foreign DNA into a host genome, e.g., at a specific site, such as a safe harbor locus. Non-limiting examples of gene editing tools and techniques include Clustered regularly interspaced short palindromic repeats (CRISPR), Transcription activator-like effector nucleases (TALEN), zinc finger nuclease (ZFN), meganuclease, Mega-TAL, and transposon-based systems. In some embodiments, an enzyme can be used that is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpfl, c2c1, c2c3, Cas9HiFi, homologues thereof, and modified versions thereof.

A CRISPR system can be utilized to facilitate insertion of a polynucleotide sequence into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III can both require pre-crRNA processing prior to assembling the processed crRNA into the multi-Cas protein complex. Types II and V CRISPR systems comprise a single Cas protein complexed with at least one guiding RNA. In some cases, a homologous recombination HR enhancer can be used to increase the efficiency of insertion of a polynucleotide and/or suppress repair of a double stranded break by non-homologous end-joining (NHEJ).

Insertion of a sequence of interest at a specific site can be promoted by recombination arms that flank the insertion site, which can, for example, promote homologous recombination at the site of a double stranded break. For example, a sequence that is to be inserted can be flanked by nucleotide sequences that are complementary to sequences flanking the targeted double strand break region in a genome. In some cases, a recombination arm can comprise a sequence that is homologous to a sequence adjacent to an insertion site that is from about 0.2 kb to about 5 kb in length. Recombination arms can be about or at least about 0.2 kb, 0.4 kb 0.6 kb, 0.8 kb, 1.0 kb, 1.2 kb, 1.4 kb, 1.6 kb, 1.8 kb, 2.0 kb, 2.2 kb, 2.4 kb, 2.6 kb, 2.8 kb, 3.0 kb, 3.2 kb, 3.4 kb, 3.6 kb, 3.8 kb, 4.0 kb, 4.2 kb, 4.4 kb, 4.6 kb, 4.8 kb, or 5 kb in length.

A transposon based system can be utilized for insertion of a polynucleic acid encoding an RNA editing entity of the disclosure or a component thereof into a genome (e.g., a piggyBAC or sleeping beauty transposon system).

In some cases, cells are genetically engineered to comprise a polynucleic acid encoding an RNA editing entity of the disclosure in vivo. In some cases, cells are genetically engineered to comprise a polynucleic acid encoding an RNA editing entity of the disclosure in vitro or ex vivo.

In some embodiments, administering the synthetic site-specific RNA editing entity reduces expression of a non-pathogenic RNA and/or protein by less than 70%, less than 65%, less than 60%, less than 55%, less than 50%, less than 45%, less than 40%, less than 35%, less than 30%, less than 25%, less than 20%, less than 10%, less than 10%, or less than 5% relative to expression of the non-pathogenic protein without administering the synthetic site-specific RNA editing entity. In some embodiments, the non-pathogenic protein is a non-pathogenic Huntingtin protein, or the non-pathogenic RNA encodes a non-pathogenic Huntingtin protein. In some embodiments, the non-pathogenic protein is an ataxin-3 protein, or the non-pathogenic RNA encodes a non-pathogenic ataxin-3 protein. In some embodiments, the non-pathogenic RNA contains a relatively low number of CAG repeats, for example, less than 40, less than 35, less than 30, less than 25, less than 20, less than 15, less than 10, or less than 5 CAG repeats. In some embodiments, the non-pathogenic RNA comprises at least 3, at least 5, at least 5, at least 6, at least 7, at least 8, at least 9, or at least 10 CAG repeats. The effect of the synthetic site-specific RNA editing entity can be determined, for example, using an in vivo, ex vivo or in vitro experimental system, such as an RT-qPCR assay, RNA seq assay, ELISA assay, or western blot assay performed on a sample from an animal administered the synthetic site-specific RNA editing entity, or cells contacted with the synthetic site-specific RNA editing entity.

In some embodiments, administering the synthetic site-specific RNA editing entity reduces expression of a pathogenic protein by at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% relative to expression of the pathogenic protein without administering the synthetic site-specific RNA editing entity. In some embodiments, the pathogenic protein is a pathogenic Huntingtin protein, pathogenic ataxin-1 protein, pathogenic ataxin-2 protein, pathogenic ataxin-3 protein, pathogenic calcium voltage-gated channel subunit alpha1 A protein, pathogenic ataxin-7 protein, pathogenic TATA-binding protein, pathogenic atrophin 1, or a pathogenic androgen receptor. In some embodiments, the pathogenic protein is a pathogenic Huntingtin protein. In some embodiments, the pathogenic protein is a pathogenic ataxin-3 protein. The effect of the synthetic site-specific RNA editing entity can be determined, for example, using an in vivo, ex vivo or in vitro experimental system, such as a western blot or ELISA assay performed on a sample from an animal administered the synthetic site-specific RNA editing entity, or cells contacted with the synthetic site-specific RNA editing entity.

In some embodiments, administering the synthetic site-specific RNA editing entity reduces expression of a pathogenic RNA by at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% relative to expression of the pathogenic RNA without administering the synthetic site-specific RNA editing entity. In some embodiments, the pathogenic RNA encodes a pathogenic Huntingtin protein, pathogenic ataxin-1 protein, pathogenic ataxin-2 protein, pathogenic ataxin-3 protein, pathogenic calcium voltage-gated channel subunit alpha1 A protein, pathogenic ataxin-7 protein, pathogenic TATA-binding protein, pathogenic atrophin 1, or a pathogenic androgen receptor. In some embodiments, the pathogenic RNA encodes a pathogenic Huntingtin protein. In some embodiments, the RNA encodes a pathogenic ataxin-3 protein. The effect of the synthetic site-specific RNA editing entity can be determined, for example, using an in vivo, ex vivo or in vitro experimental system, such as an RT-qPCR assay or RNA seq assay performed on a sample from an animal administered the synthetic site-specific RNA editing entity, or cells contacted with the synthetic site-specific RNA editing entity.

In some embodiments, administering the synthetic site-specific RNA editing entity reduces total expression of an RNA and/or protein, for example, an aggregate of combined expression of a non-pathogenic allele and a pathogenic allele, by at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% relative to total expression of the RNA and/or protein without administering the synthetic site-specific RNA editing entity. In some embodiments, the RNA encodes and/or the protein is a Huntingtin protein, ataxin-1 protein, ataxin-2 protein, ataxin-3 protein, calcium voltage-gated channel subunit alpha1A protein, ataxin-7 protein, TATA-binding protein, atrophin 1, or an androgen receptor. In some embodiments, the RNA encodes and/or the protein is a Huntingtin protein. In some embodiments, the RNA encodes and/or the protein is an ataxin-3 protein. The effect of the synthetic site-specific RNA editing entity can be determined, for example, using an in vivo, ex vivo or in vitro experimental system, such as an RT-qPCR assay, an RNA seq assay, an ELISA assay, or a western blot assay performed on a sample from an animal administered the synthetic site-specific RNA editing entity, or cells contacted with the synthetic site-specific RNA editing entity.

In some embodiments, an effect of an ASRE disclosed herein on expression of a pathogenic and/or non-pathogenic RNA and/or protein can be determined by an RT-qPCR assay (e.g., as disclosed herein). In some embodiments, an effect of an ASRE disclosed herein on expression of a pathogenic and/or non-pathogenic RNA and/or protein can be determined by a western blot assay (e.g., as disclosed herein). In some embodiments, an effect of an ASRE disclosed herein on expression of a pathogenic and/or non-pathogenic RNA and/or protein can be determined in a cell culture model, for example, utilizing cells from patients with a CAG repeat disorder, or utilizing cells engineered to express a pathogenic RNA and protein (e.g., pathogenic Htt) and control cells optionally engineered to express a corresponding non-pathogenic RNA and protein.

In some embodiments, the dosage of the pharmaceutical compositions depends on factors including the route of administration, the disease to be treated, and physical characteristics, e.g., age, weight, general health, of the subject. In some embodiments, the amount of the pharmaceutical composition contained within a single dose is an amount that effectively prevents, delays, or treats the disease without inducing significant toxicity. In some embodiments, the effective amount for use in humans is determined from animal models. In some embodiments, a dose for humans is formulated to achieve a concentration in CSF that has been found to be effective and/or non-toxic or minimally-toxic in animals. In some embodiments, a dose for humans is formulated to achieve circulating, liver, topical, and/or gastrointestinal concentrations that have been found to be effective and/or non-toxic or minimally-toxic in animals. In some embodiments, the dosage is adapted by the medical professional in accordance with conventional factors such as the extent of the disease and different parameters of the subject. In some embodiments, the composition is administered before, during, or after the onset of a symptom associated with the CAG repeat neurodegenerative condition.

In some embodiments, the composition and kit described herein are stored at between 2° C. and 8° C. In some embodiments, the composition is not stored frozen. In some embodiments, the composition is stored at a temperature at or below 0° C. In some instances, the immunotherapeutic composition is stored in temperatures of between −20° C. or −80° C.

### Methods of Production

Described herein, in certain embodiments, are methods of producing a site-specific RNA editing entity targeting a pathogenic RNA comprising a CAG repeat. In some embodiments, the method comprises generating a polynucleotide encoding a variant of the human Pumilio 1 homology (PUF) domain effective to bind a ten nucleotide RNA target sequence selected from the group consisting of: CAGCAGCAGC (SEQ ID NO: 28), AGCAGCAGCA (SEQ ID NO: 29), and GCAGCAGCAG (SEQ ID NO: 30). In some embodiments, the method comprises generating a polynucleotide encoding a variant of the human Pumilio 1 homology (PUF) domain effective to bind an eight nucleotide RNA target sequence selected from the group consisting of: CAGCAGCA, AGCAGCAG, and GCAGCAGC. In some embodiments, the polynucleotide is generated by gene synthesis. In some embodiments, gene synthesis comprises use of short oligonucleotides to generate the polynucleotide sequence. In some embodiments, the method further comprises expressing, in a cell, a recombinant vector comprising the polynucleotide sequence. In some embodiments, the polynucleotide sequence is codon optimized.

In some embodiments, prior to the expressing, the method comprises introducing a recombinant vector into the cell. In some embodiments, the recombinant vector comprises a non-viral vector. In some embodiments, the non-viral vector comprises a plasmid DNA, a minicircle DNA, a liposome-DNA complex (e.g., lipoplex), or a polymer-DNA complex (e.g., polyplex). In some embodiments, the introducing comprises transfection. In some embodiments, the transfection is achieved through lipid mediated delivery. In some embodiments, the transfection requires the use of a transfection agent. In some embodiments, the transfection agent is Oligofectamine™ or Lipofectamine™. In some embodiments, the recombinant vector comprises a viral vector. Any suitable viral vector can be used to introduce the synthetic site-specific RNA editing entity into a cell described herein, including, but not limited to a retroviral vector, an adenoviral vector (e.g., Adenovirus type 5), an adeno associated virus (AAV) vector, an alphavirus vector, a vaccinia virus vector, a herpes simplex virus (HSV) vector, a lentivirus vector, or a retrovirus vector.

Disclosed herein, in some embodiments are methods reducing a pathogenic RNA comprising a CAG repeat and/or a pathogenic protein encoded by an RNA that comprises a CAG repeat (e.g., CAG repeat expansion) in a cell. In some embodiments, the methods comprise introducing to the cell a synthetic site-specific RNA editing entity comprising an RNA binding protein specific to a CAG repeat in the pathogenic RNA or an RNA that encodes the pathogenic protein expressed by the cell. In some embodiments, the reduction in pathogenic RNA is measured by an assay comprising reverse transcription polymerase chain reaction (RT-PCR). In some embodiments, the reduction in the pathogenic protein is measured by a western blot or ELISA assay. In some embodiments, the synthetic site-specific RNA editing entity is introduced to the cell with an efficiency of at least about 70%, 75%, 80%, 81%, 82%, 83%, 84%, 85%, 86%, 87%, 88%, 89%, 90%, 91%, 92%, 93%, 94%, 95%, 96%, 97%, 99%, or 100%, when normalized to glyceraldehyde-3-phosphate dehydrogenase (GAPDH) expression in the cell. In some embodiments the efficiency is measured when the multiplicity of infection (MOI) comprises a range between about 50 and 1100, 100 and 1000, 200 and 900, 300 and 800, 400 and 700, or 500 and 600. In some embodiments the efficiency is measured when MOI is less than or equal to about 50, 100, 200, 300, 400, 500, 600, 700, 800, 900, or 1000.

In some embodiments, the CAG repeat is associated with, or causes, a CAG repeat disorder, such as a CAG repeat expansion disorder. In some embodiments, the CAG repeat disorder comprises Huntington's disease (HD), spinocerebellar ataxia (SCA), dentatorubral-pallidoluysian atrophy (DRPLA), or spinal and bulbar muscular atrophy (SBMA). In some embodiments, the SCA comprises or the SCA is SCA1, SCA2, SCA3 (Machado-Joseph disease), SCA6, SCAT, or SCA17. In some embodiments, the synthetic site-specific RNA editing entity comprises an RNA binding domain that specifically binds to a target RNA sequence in a pathogenic RNA or in an RNA encoding the pathogen protein in the cell. In some embodiments, the target RNA sequence is an 8mer, 9 mer, 10mer, 11mer, 12mer, 13mer, 14mer, 15mer, or 16mer. In some embodiments, the target RNA sequence is an 8mer. In some embodiments, the target RNA sequence is a 10mer. In some embodiments, the target RNA sequence is or comprises CAGCAGCAGC (SEQ ID NO: 28), AGCAGCAGCA (SEQ ID NO: 29), or GCAGCAGCAG (SEQ ID NO: 30), CAGCAGCA, AGCAGCAG, or GCAGCAGC. In some embodiments, the pathogenic protein comprises a pathogenic Huntingtin (Htt) protein, a pathogenic ataxin-1 (ATXN1) protein, a pathogenic ataxin-2 (ATXN2) protein, a pathogenic ataxin-3 (ATXN3) protein, a pathogenic calcium voltage-gated channel subunit alpha1A (CACNA1A), a pathogenic ataxin-7 (ATXN7) protein, a pathogenic TATA-binding protein (TBP), a pathogenic atrophin 1 (ATN1), a pathogenic androgen receptor (AR), or any combination thereof. In some embodiments, the synthetic site-specific RNA editing entity is introduced into the cell by a vector comprising a viral or non-vial vector, such as those described elsewhere herein.

In some embodiments, the cell is a bacterial cell. In some embodiments, the bacterial cell is Escherichia coli. In some embodiments, the cell is part of a cell culture. In some embodiments, the cell culture is a production cell line. In some embodiments, the production cell line is a non-human production cell line. Non-human production cell lines include, but are not limited to, Chinese hamster ovary (CHO) cells, baby hamster kidney (BHK21) cells, or murine myeloma cells (NS0 and Sp2/0). In some embodiments, the production cell line is a bacterial cell line. In some embodiments, the bacterial cell line is an E. coli cell line. In some embodiments, the production cell line is a human production cell line. Human production cell lines include, but are not limited to, HEK293, HT-1080, PER.C6, CAP, HKB-11, and HuH-7. In some embodiments, the cell is a yeast. In some embodiments, the yeast is Saccharomyces cerevisiae.

In some embodiments, the method comprises expanding the cell to produce a plurality of expanded cells. In some embodiments, the expanding occurs in a bioreactor. In some embodiments, the bioreactor is a stirred suspension bioreactor. In some embodiments, the method comprises isolating the site-specific RNA editing entity after the expanding. In some embodiments, the method comprises purifying the site-specific RNA editing entity.

### EMBODIMENTS

Embodiment 1. A synthetic RNA binding domain comprising an amino acid sequence with at least 90% sequence identity to SEQ ID NO: 6.
Embodiment 2. A synthetic RNA binding domain comprising an amino acid sequence with at least 95% sequence identity to SEQ ID NO: 10.
Embodiment 3. A synthetic RNA binding domain that targets a pathogenic RNA comprising a CAG repeat, the synthetic RNA binding domain comprising an amino acid sequence comprising (Cys/Ser/Asn)XxxXxxXxxGln that binds to adenine, wherein Xxx is any amino acid.
Embodiment 4. A synthetic site-specific RNA editing entity targeting a pathogenic RNA that comprises a CAG repeat, the site-specific RNA editing entity comprising: (i) a synthetic RNA binding domain; and (ii) a cleavage domain; wherein the synthetic RNA binding domain comprises an amino acid sequence comprising (Cys/Ser/Asn)XxxXxxXxxGln that binds to adenine, wherein Xxx is any amino acid.
Embodiment 5. A synthetic site-specific RNA editing entity targeting a pathogenic RNA that comprises a CAG repeat, the site-specific RNA editing entity comprising: (i) a synthetic RNA binding domain comprising an amino acid sequence with at least 90% sequence identity to SEQ ID NO: 6; and (ii) a cleavage domain.
Embodiment 6. A synthetic site-specific RNA editing entity targeting a pathogenic RNA that comprises a CAG repeat, the site-specific RNA editing entity comprising: (i) a synthetic RNA binding domain comprising an amino acid sequence with at least 95% sequence identity to SEQ ID NO: 10; and (ii) a cleavage domain.
Embodiment 7. A method of treating a subject in need thereof, comprising administering to the subject a synthetic site-specific RNA editing entity targeting a pathogenic RNA that comprises a CAG repeat, the site-specific RNA editing entity comprising: (i) a synthetic RNA binding domain; and (ii) a cleavage domain; wherein the synthetic RNA binding domain comprises an amino acid sequence comprising (Cys/Ser/Asn)XxxXxxXxxGln that binds to adenine, wherein Xxx is any amino acid.
Embodiment 8. A method of treating a subject in need thereof, comprising administering to the subject a synthetic site-specific RNA editing entity targeting a pathogenic RNA that comprises a CAG repeat, the site-specific RNA editing entity comprising: (i) a synthetic RNA binding domain comprising an amino acid sequence with at least 90% sequence identity to SEQ ID NO: 6; and (ii) a cleavage domain.
Embodiment 9. A method of treating a subject in need thereof, comprising administering to the subject a synthetic site-specific RNA editing entity targeting a pathogenic RNA that comprises a CAG repeat, the site-specific RNA editing entity comprising: (i) a synthetic RNA binding domain comprising an amino acid sequence with at least 95% sequence identity to SEQ ID NO: 10; and (ii) a cleavage domain.
Embodiment 10. A synthetic site-specific RNA editing entity targeting a pathogenic RNA that comprises a CAG repeat, the site-specific RNA editing entity comprising: (i) a synthetic RNA binding domain; and (ii) a cleavage domain that comprises a PilT N-terminus (PIN) domain or an enzymatically-active variant, derivative, or fragment thereof
Embodiment 11. A synthetic site-specific RNA editing entity targeting a pathogenic RNA that comprises a CAG repeat, the site-specific RNA editing entity comprising: (i) a synthetic RNA binding domain that comprises an engineered human Pumilio 1 domain; and (ii) a cleavage domain.
Embodiment 12. A synthetic site-specific RNA editing entity having a formula B-L-C, wherein B is a synthetic RNA binding domain that specifically binds to one or more repeats of a CAG nucleotide sequence, L is a synthetic linker, and C is a cleavage domain.
Embodiment 13. A cell comprising a pathogenic RNA that comprises a CAG repeat, and a synthetic site-specific RNA editing entity capable of cleaving, modifying, editing, or modulating expression of the pathogenic RNA, the synthetic site-specific RNA editing entity having a formula B-L-C, wherein B is a synthetic RNA binding domain that specifically binds to one or more repeats of a CAG nucleotide sequence, L is a synthetic linker, and C is a cleavage domain.
Embodiment 14. A method of reducing a level of a pathogenic RNA that comprises a CAG repeat or a translation product thereof, comprising contacting the pathogenic RNA with a synthetic site-specific RNA editing entity having a formula B-L-C, wherein B is a synthetic RNA binding domain that specifically binds to one or more repeats of a CAG nucleotide sequence, L is a synthetic linker, and C is a cleavage domain.
Embodiment 15. Any of the preceding embodiments, wherein the synthetic RNA binding domain comprises at least one mutation at a position corresponding to residues 36-362 of SEQ ID NO: 6.
Embodiment 16. Any of the preceding embodiments, wherein the synthetic RNA binding domain comprises at least one mutation at a position corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; residues 358 to 362 of SEQ ID NO: 6; or any combination of (a) to (j).
Embodiment 17. Any of the preceding embodiments, wherein the synthetic RNA binding domain comprises at least one mutation in at least two ranges of residues corresponding to: residues 36 to 40 of SEQ ID NO: 6; residues 72 to 76 of SEQ ID NO: 6; residues 108 to 112 of SEQ ID NO: 6; residues 144 to 148 of SEQ ID NO: 6; residues 178 to 182 of SEQ ID NO: 6; residues 214 to 218 of SEQ ID NO: 6; residues 250 to 254 of SEQ ID NO: 6; residues 286 to 290 of SEQ ID NO: 6; residues 322 to 326 of SEQ ID NO: 6; or residues 358 to 362 of SEQ ID NO: 6.
Embodiment 18. Any of the preceding embodiments, wherein the synthetic RNA binding domain facilitates cleavage of an RNA comprising a CAG repeat by a synthetic site-specific RNA editing entity, when the synthetic RNA binding domain is present in the synthetic site-specific RNA editing entity and is associated with the RNA.
Embodiment 19. Any of the preceding embodiments, wherein the CAG repeat comprises a nucleotide sequence that is CAGCAGCAGC (SEQ ID NO: 28), AGCAGCAGCA (SEQ ID NO: 29), GCAGCAGCAG (SEQ ID NO: 30), or any combination thereof
Embodiment 20. Any of the preceding embodiments, wherein the synthetic RNA binding domain comprises an amino acid sequence with at least 90% sequence identity to any one of SEQ ID NOs: 7-9.
Embodiment 21. Any of the preceding embodiments, wherein the synthetic RNA binding domain comprises an amino acid sequence with at least 95%, at least 97%, at least 98%, or at least 99% sequence identity to any one of SEQ ID NOs: 7-9.
Embodiment 22. Any of the preceding embodiments, wherein the synthetic RNA binding domain comprises an amino acid sequence that is any one of SEQ ID NOs: 7-9.
Embodiment 23. Any of the preceding embodiments, wherein the synthetic RNA binding domain comprises at least one mutation at a position corresponding to residues 36-290 of SEQ ID NO: 10.
Embodiment 24. Any of the preceding embodiments, wherein the synthetic RNA binding domain comprises at least one mutation at a position corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; residues 286 to 290 of SEQ ID NO: 10; or any combination of (a) to (h).
Embodiment 25. Any of the preceding embodiments, wherein the synthetic RNA binding domain comprises at least one mutation in at least two ranges of residues corresponding to: residues 36 to 40 of SEQ ID NO: 10; residues 72 to 76 of SEQ ID NO: 10; residues 108 to 112 of SEQ ID NO: 10; residues 144 to 148 of SEQ ID NO: 10; residues 180 to 184 of SEQ ID NO: 10; residues 214 to 218 of SEQ ID NO: 10; residues 250 to 254 of SEQ ID NO: 10; or residues 286 to 290 of SEQ ID NO: 10.
Embodiment 26. Any of the preceding embodiments, wherein the synthetic RNA binding domain facilitates cleavage of an RNA comprising a CAG repeat by a synthetic site-specific RNA editing entity, when the synthetic RNA binding domain is present in the synthetic site-specific RNA editing entity and is associated with the RNA.
Embodiment 27. Any of the preceding embodiments, wherein the CAG repeat comprises a nucleotide sequence that is CAGCAGCA, AGCAGCAG, GCAGCAGC, or any combination thereof
Embodiment 28. Any of the preceding embodiments, wherein the RNA comprising the CAG repeat is messenger RNA or pre-messenger RNA.
Embodiment 29. Any of the preceding embodiments, wherein the synthetic RNA binding domain comprises an amino acid sequence with at least 92% sequence identity to any one of SEQ ID NOs: 11-13 and 44.
Embodiment 30. Any of the preceding embodiments, wherein the synthetic RNA binding domain comprises an amino acid sequence with at least 95%, at least 97%, at least 98%, or at least 99% sequence identity to any one of SEQ ID NOs: 11-13 and 44.
Embodiment 31. Any of the preceding embodiments, wherein the synthetic RNA binding domain comprises an amino acid sequence that is any one of SEQ ID NOs: 11-13 and 44.
Embodiment 32. Any of the preceding embodiments, wherein the at least one mutation results in the synthetic RNA binding domain that has an amino acid sequence comprising SerTyrXxxXxxArg that binds cytosine, wherein Xxx is any amino acid.
Embodiment 33. Any of the preceding embodiments, wherein the at least one mutation results in the synthetic RNA binding domain that has an amino acid sequence comprising SerXxxXxxXxxGlu that binds guanine, wherein Xxx is any amino acid.
Embodiment 34. A composition comprising an isolated and purified RNA editing entity that comprises the synthetic RNA binding domain of any one of the preceding embodiments.
Embodiment 35. A polynucleotide sequence encoding the synthetic RNA binding domain of any of the preceding embodiments.
Embodiment 36. Any of the preceding embodiments, wherein the synthetic RNA binding domain comprises an amino acid sequence with at least 90% sequence identity to SEQ ID NO: 6.
Embodiment 37. Any of the preceding embodiments, wherein the synthetic RNA binding domain comprises at least one mutation at a position corresponding to residues 36-362 of SEQ ID NO: 6.
Embodiment 38. Any of the preceding embodiments, wherein the synthetic site-specific RNA editing entity facilitates cleavage of the pathogenic RNA that comprises the CAG repeat when the synthetic site-specific RNA editing entity is associated with the pathogenic RNA.
Embodiment 39. Any of the preceding embodiments, wherein the CAG repeat comprises a nucleotide sequence that is CAGCAGCAGC (SEQ ID NO: 28), AGCAGCAGCA (SEQ ID NO: 29), GCAGCAGCAG (SEQ ID NO: 30), or any combination thereof
Embodiment 40. Any of the preceding embodiments, wherein the cleavage domain cleaves upstream or downstream of a 10 nucleotide RNA target sequence.
Embodiment 41. Any of the preceding embodiments, wherein the synthetic RNA binding domain comprises an amino acid sequence with at least 95% sequence identity to SEQ ID NO: 10.
Embodiment 42. Any of the preceding embodiments, wherein the synthetic RNA binding domain comprises an engineered human Pumilio 1 domain.
Embodiment 43. Any of the preceding embodiments, wherein the synthetic RNA binding domain comprises an amino acid sequence with at least 92% sequence identity to any one of SEQ ID NOs: 35-37.
Embodiment 44. Any of the preceding embodiments, wherein the synthetic RNA binding domain comprises an amino acid sequence with at least 95%, at least 97%, at least 98%, or at least 99% sequence identity to any one of SEQ ID NOs: 35-37.
Embodiment 45. Any of the preceding embodiments, wherein the synthetic RNA binding domain comprises an amino acid sequence that is any one of SEQ ID NOs: 35-37.
Embodiment 46. Any of the preceding embodiments, wherein the synthetic site-specific RNA editing entity has RNA endonuclease activity.
Embodiment 47. Any of the preceding embodiments, wherein the cleavage domain comprises a PilT N-terminus (PIN) domain or an enzymatically-active variant, derivative, or fragment thereof
Embodiment 48. Any of the preceding embodiments, wherein the cleavage domain comprises a PilT N-terminus (PIN) domain of human SMG6.
Embodiment 49. Any of the preceding embodiments, wherein the at least one mutation results in the synthetic RNA binding domain that has an amino acid sequence comprising SerTyrXxxXxxArg that binds cytosine, wherein Xxx is any amino acid.
Embodiment 50. Any of the preceding embodiments, wherein the at least one mutation results in the synthetic RNA binding domain that has an amino acid sequence comprising (Cys/Ser/Asn)XxxXxxXxxGln that binds to adenine, wherein Xxx is any amino acid.
Embodiment 51. Any of the preceding embodiments, wherein the at least one mutation results in the synthetic RNA binding domain that has an amino acid sequence comprising SerXxxXxxXxxGlu that binds to guanine, wherein Xxx is any amino acid.
Embodiment 52. Any of the preceding embodiments, wherein the cleavage domain comprises an amino acid sequence with at least 95%, at least 97%, at least 98%, or at least 99% sequence identity to SEQ ID NO: 41.
Embodiment 53. Any of the preceding embodiments, wherein the cleavage domain comprises an amino acid sequence that is SEQ ID NO: 41.
Embodiment 54. Any of the preceding embodiments, wherein a C-terminus of the synthetic RNA binding domain is joined to an N-terminus of the cleavage domain.
Embodiment 55. Any of the preceding embodiments, wherein an N-terminus of the synthetic RNA binding domain is joined to a C-terminus of the cleavage domain.
Embodiment 56. Any of the preceding embodiments, further comprising a linker.
Embodiment 57. Any of the preceding embodiments, wherein a C-terminus of the synthetic RNA binding domain is joined to an N-terminus of a linker and a C-terminus of the linker is joined to an N-terminus of the cleavage domain.
Embodiment 58. Any of the preceding embodiments, wherein a N-terminus of the synthetic RNA binding domain is joined to a C-terminus of a linker and a N-terminus of the linker is joined to a C-terminus of the cleavage domain.
Embodiment 59. Any of the preceding embodiments, wherein the linker is at least three amino acids in length and at most twenty amino acids in length.
Embodiment 60. Any of the preceding embodiments, wherein the linker comprises an amino acid sequence from Table 1.
Embodiment 61. Any of the preceding embodiments, wherein the linker comprises an amino acid sequence that is VDTANGS (SEQ ID NO: 42).
Embodiment 62. A composition comprising an isolated and purified synthetic site-specific RNA editing entity of any one of the preceding embodiments.
Embodiment 63. A polynucleotide sequence encoding the synthetic site-specific RNA editing entity of any one of the preceding embodiments.
Embodiment 64. A vector comprising a polynucleotide sequence encoding the synthetic site-specific RNA editing entity of any one of the preceding embodiments.
Embodiment 65. The vector of embodiment 64, wherein the vector is a viral vector
Embodiment 66. The vector of embodiment 64, wherein the vector is an adeno-associated viral vector (AAV), retroviral vector, adenoviral vector, or a lentiviral vector.
Embodiment 67. A pharmaceutical composition comprising the vector, composition, or synthetic site-specific RNA editing entity of any one of the preceding embodiments and a pharmaceutically acceptable excipient, carrier, or diluent.
Embodiment 68. A kit comprising the vector, composition, or synthetic site-specific RNA editing entity of any one of the preceding embodiments.
Embodiment 69. A cell or cell culture expressing the synthetic site-specific RNA, the synthetic RNA binding domain, or the polynucleotide sequence of any of the preceding embodiments.
Embodiment 70. A method of delivering a synthetic site-specific RNA editing entity to a cell, comprising administering to the cell the vector of any preceding embodiment, optionally wherein the polynucleotide sequence encoding the synthetic site-specific RNA editing entity is integrated into the genome of the cell.
Embodiment 71. Any of the preceding embodiments, wherein the subject has a CAG repeat-associated disorder.
Embodiment 72. Any of the preceding embodiments, wherein the subject has a CAG repeat-associated neurological disorder.
Embodiment 73. Any of the preceding embodiments, wherein the subject has a CAG repeat-associated neurodegenerative disorder.
Embodiment 74. Any of the preceding embodiments, wherein the subject has Huntington's disease (HD), spinocerebellar ataxia (SCA), dentatorubral-pallidoluysian atrophy (DRPLA), or spinal and bulbar muscular atrophy (SBMA).
Embodiment 75. Any of the preceding embodiments, wherein the subject has Huntington's disease (HD).
Embodiment 76. Any of the preceding embodiments, wherein administering the synthetic site-specific RNA editing entity or the vector reduces expression of the pathogenic Huntingtin protein by 60% or more relative to expression of the pathogenic Huntingtin protein without the administering.
Embodiment 77. Any of the preceding embodiments, wherein administering the synthetic site-specific RNA editing entity or the vector reduces expression of a wild-type Huntingtin protein by 40% or less relative to expression of the wild-type Huntingtin protein without the administering.
Embodiment 78. Any of the preceding embodiments, wherein the subject has spinocerebellar ataxia (SCA) type 1, SCA type 2, SCA type 3, SCA type 6, SCA type 7, or SCA type 17.
Embodiment 79. The preceding embodiments, wherein the subject has the SCA type 3.
Embodiment 80. Any of the preceding embodiments, further comprising administering an additional therapeutic agent to the subject.
Embodiment 81. The preceding embodiment, wherein the additional therapeutic agent is an antipsychotic, a drug to treat chorea, an antidepressant, a mood-stabilizing drug, an anti-inflammatory drug, a neuroprotective drug, or a combination thereof
Embodiment 82. Any of the preceding embodiments, wherein the administering comprises parenteral administration.
Embodiment 83. Any of the preceding embodiments, wherein the administering comprises intracranial injection or intrathecal injection.
Embodiment 84. A method of producing a synthetic site-specific RNA editing entity that targets a pathogenic RNA comprising a CAG repeat, the method comprising expressing the synthetic site-specific RNA editing entity of any preceding embodiment in a cell, and harvesting the synthetic site-specific RNA editing entity.
Embodiment 85. Any of the preceding embodiments, wherein cell is a bacterium.
Embodiment 86. Any of the preceding embodiments, wherein bacterium is Escherichia coli.
Embodiment 87. Any of the preceding embodiments, wherein the cell is a yeast
Embodiment 88. Any of the preceding embodiments, wherein the yeast is Saccharomyces cerevisiae.
Embodiment 89. Any of embodiments 12-88, wherein the synthetic linker comprises an amino acid sequence that is heterologous to amino acid sequences of the synthetic RNA binding domain and the cleavage domain.
Embodiment 90. Any of embodiments 12-88, wherein the synthetic linker comprises an amino acid sequence from table 1.

### Certain Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition including a compound as disclosed herein required to provide a clinically significant decrease in disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that “an effective amount” or “a therapeutically effective amount” can vary from subject to subject, due to variation in metabolism of the composition, age, weight, general condition of the subject, the condition being treated, the severity of the condition being treated, and the judgment of the prescribing physician.

As used herein, the terms “subject,” “individual” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and compositions provided herein, the mammal is a human.

As used herein, the terms “treat,” “treating” or “treatment,” and other grammatical equivalents, include alleviating, abating or ameliorating one or more symptoms of a disease or condition, ameliorating, preventing or reducing the appearance, severity or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, or inhibiting the symptoms of the disease or condition either prophylactically and/or therapeutically. In a non-limiting example, for prophylactic benefit, a synthetic site-specific RNA editing entities or composition disclosed herein is administered to a subject at risk of developing a particular disorder, predisposed to developing a particular disorder, or to a subject reporting one or more of the physiological symptoms of a disorder.

As used herein, an “RNA editing entity” refers to an enzymatically active entity capable of editing an RNA sequence or molecule. In some embodiments, the RNA editing entity has endonuclease activity. In some embodiments, the RNA editing entity is an endonuclease, or enzymatically active fragment of an endonuclease.

### EXAMPLES

### Example 1: Engineer ASREs that Specifically Recognize Expanded CAG Repeats

PUF domains that recognize specific RNA target sequences are designed. A yeast three-hybrid system is used to design CAG specific PUF domains that bind 8-nt RNA target sequences and 10-nt RNA target sequences in each of the three frames of the (CAG)n repeat (SEQ ID NOs: 28-30), resulting in separate ASREs that target each sequence.

The coding sequences of each identified PUF domain is fused in frame with the PIN RNA endonuclease to create a sequence that encodes a synthetic site-specific RNA editing entity, ASRE(CAG)ₙ, optionally with a FLAG tag included (e.g., SEQ ID NO: 45).

The ASRE-encoding sequences are then cloned into piggyBac (PB) transposon expression vectors to generate PB-ASRE(CAG)n.

Separately, the ASRE-encoding sequences are packaged in Adenoviral-5 (Ad5) vectors to generate Ad5-ASRE(CAG)ₙ.

### Example 2: Efficacy of (CAG)n Specific ASREs for Reducing Levels of Mutant Huntingtin RNA and Protein in a Mouse Embryonic Stem Cell Model

Mouse ES cell line model of Huntington's Disease: A mouse embryonic stem (ES) cell model is utilized to evaluate efficacy of ASRE(CAG), for reducing levels of pathogenic mutant Huntingtin RNA and protein. The cells are engineered to express one copy of Huntingtin that contains a human exon 1.

As a model of cells that express pathogenic mutant Huntingtin RNA and protein, mouse ES cell lines are utilized that express a copy of Huntingtin that contains a pathogenic human exon 1 sequence, with 140 copies of the CAG repeat (SEQ ID NO: 111) (Htt^{140Q}).

As a control, mouse ES cell lines are utilized that express a copy of Huntingtin that contains a normal human exon 1 sequence, with 20 copies of the CAG repeat (SEQ ID NO: 112) (Htt^{20Q}).

The copies of Huntingtin that contain the human exon 1 sequence can also encode an epitope tag (e.g., a 3X FLAG tag) to enable detection by an anti-FLAG antibody.

Both cell lines are heterozygous, with the second allele containing a mouse Huntingtin sequence including exon 1, which contains 7 CAG repeats (SEQ ID NO: 113) (Htt^{140Q/7Q} and Htt^{20Q/7Q}). The ES cell lines are generated using standard gene targeting methods. The knock-in and endogenous wild type mouse Htt alleles can be expressed at similar levels, thereby providing a stringent test for the ability of the ASRE to specifically cleave the expanded Htt^{140Q} allele RNA transcripts.

The Htt^{140Q/7Q} and Htt^{20Q/7Q} ES cells can also be differentiated into neurons and used to quantitate the level of 20 Q-Htt and 140 Q-Htt mRNA knockdown.

piggyBAC and Adenovirus-5 vectors are used to deliver ASREs of the disclosure to the cells, e.g., CAG8, AGC8, GCA8, CAG10, AGC10, and/or GCA10 ASREs.

ES cell transduction with PB-ASRE(CAG)ₙ: Each piggyBAC ASRE(CAG)n construct is co-transfected with PB transposase into the ES cells, for example, using lipid-mediated transfection. Cells are transduced using DNA concentrations that favor single transposon integration events, and plated to select for puromycin resistance, which is a selectable marker present in the PB transposon. Ratios of vector to transposase are utilized to obtain 1-2 integrants per drug resistant clone. Puromycin-resistant ES cell clones are picked for expansion, cryopreservation, and Western blotting to ensure the ASRE(CAG) fusion protein is expressed. The ES cells are transfected with the piggyBAC ASRE transposon and transposase vectors to enable selection of individual clones containing both the integrated ASRE and the knock-in Htt allele. ASRE⁺ (i.e., puromycin resistant) clones from the transduced experimental (Htt^{140Q/7Q}) and control (Htt^{20Q/7Q}) ES cells are screened for integration of the ASRE construct. Independent clones from each group are used for ASRE expression. Each ES cell clone is expanded and cultured for 72 h. ES cell transduction with Ad5-ASRE(CAG)n: Experimental (Htt^{140Q/7Q}) and control (Htt^{20Q/7Q}) ES cells are transduced with each adenoviral-5 ASRE(CAG)n construct at multiplicities of infection (MOI) ranging from 400 to 1000.

Quantification of levels of pathogenic Huntingtin RNA: The level of knockdown of the human 20 Q-Htt and 140 Q-Htt mRNA relative to the normal mouse 7 Q-Htt mRNA is assessed by RT-qPCR. Comparisons are performed 3-7 days after the ES cells are transduced with the Ad5-ASREs or PB-ASREs. Total RNA is isolated from the cultures, and RT-qPCR is performed to quantify the relative levels of 140 Q to 7 Q and 20 Q to 7 Q RNA in each culture condition. Each clone is analyzed in triplicate.

Quantification of levels of pathogenic Huntingtin protein: The level of knockdown of the expanded polyQ mutant Huntingtin (mHtt) protein relative to normal Htt (wtHtt) is assessed by western blotting. Comparisons are performed 3-7 days after the ES cells are transduced with the Ad5-ASREs or PB-ASREs. Whole ES cell protein lysates are prepared from the cultures, and 60 mg of each sample is analyzed by western blotting. A primary antibody recognizing both human and mouse Htt is used to detect total Htt (MAB2166, Chemicon; and D7F7, Epitomics). A primary antibody that recognizes the human Htt proline-rich region is used to detect levels of 140 Q-Htt and 20 Q-Htt (MAb 5492, Millipore). Alternatively or additionally, for FLAG tagged Htt, an anti-FLAG primary antibody can be used. An antibody recognizing total mTOR (2972S, Cell Signaling) is used to normalize protein loading in each lane. Blots are imaged and quantified using near-IR fluorescent secondary antibodies in a LiCor Odyssey Fc with Image Studio software.

The effect of the ASREs on levels of total Htt, 140 Q-Htt, and 20 Q-Htt, and associated RNAs are evaluated.

### Example 3: Efficacy of (CAG)n Specific ASREs for Reducing Levels of Mutant Htt RNA and Protein in Primary Human Fibroblasts from Huntington's Disease (HD) Patients

Primary human fibroblasts are obtained from patients with Huntington's disease (HD). Huntington's disease is associated with pathogenic versions of Huntingtin protein (Htt) encoded by RNAs that contain a higher number of CAG repeats than RNAs that encode non-pathogenic versions of Huntingtin protein.

The impact of ASREs of the disclosure on levels of Huntingtin RNA and protein can be evaluated for primary human cells from subjects with Huntington's disease, and/or primary human fibroblasts from human subjects with normal Huntingtin can be used as controls.

piggyBAC and Adenovirus-5 vectors are used to deliver ASREs of the disclosure to the cells, e.g., CAG8, AGC8, GCA8, CAG10, AGC10, and/or GCA10 ASREs.

Fibroblast transduction with PB-ASRE(CAG)n: Each piggyBAC (PB) ASRE(CAG)ₙ construct is co-transfected with PB transposase into the fibroblasts. Cells are transduced using DNA concentrations that favor single transposon integration events, and plated to select for puromycin resistance, which is a selectable marker present in the PB transposon. Ratios of vector to transposase are utilized to obtain 1-2 integrants per drug resistant clone. Puromycin-resistant fibroblast cell clones are picked for expansion, cryopreservation, and Western blotting to ensure the ASRE(CAG) fusion protein is expressed. ASRE⁺ (i.e., puromycin resistant) clones from the transduced experimental (mutant Htt) and control (normal Htt) fibroblasts are screened for integration of the ASRE construct. Independent clones from each group are used for ASRE expression. Each fibroblast cell clone is expanded and cultured for 72 h.

Fibroblast transduction with Ad5-ASRE(CAG)ₙ: Experimental (mutant Htt) and control (normal Htt) fibroblasts are transduced with each adenoviral-5 ASRE(CAG)n construct at multiplicities of infection (MOI) ranging from 400 to 1000.

Quantification of levels of pathogenic Huntingtin RNA: The level of knockdown of the pathogenic/normal Huntingtin mRNA is assessed by RT-qPCR. Comparisons are performed 3-7 days after the fibroblasts are transduced with the Ad5-ASREs or PB-ASREs (e.g., day 3). Total RNA is isolated from the cultures, and RT-qPCR is performed to quantify the relative levels of pathogenic/normal Huntingtin in each culture condition. Each clone is analyzed in triplicate.

Quantification of levels of pathogenic Huntingtin protein: Levels of knockdown of the pathogenic and normal Huntingtin proteins are assessed by western blotting. Comparisons are performed 3-7 days after the fibroblasts are transduced with the Ad5-ASREs or PB-ASREs (e.g., day 7). Whole cell protein lysates are prepared from the cultures, and 60 mg of each sample is analyzed by western blotting. Primary antibodies are used to identify normal Huntingtin and pathogenic Huntingtin, and a loading control. Blots are imaged and quantified using near-IR fluorescent secondary antibodies in a LiCor Odyssey Fc with Image Studio software. The effect of the ASREs on levels of pathogenic and normal Huntingtin protein and RNA are evaluated.

### Example 4: Efficacy of (CAG)n Specific ASREs for Reducing Levels of Mutant Ataxin-3 RNA and Protein in Primary Human Fibroblasts from SCA3 (Machado-Joseph Disease) Patients

Primary human fibroblasts are obtained from patients with spinocerebellar ataxia type 3 (SCA3, Machado-Joseph disease). SCA3 is caused by a pathogenic ataxin-3 (ATXN3) protein, which can be encoded by a pathogenic RNA that comprises a higher number of CAG repeats than an RNA encoding a non-pathogenic ATXN3.

Levels of ATXN3 with and without ASREs can be compared, and/or primary human fibroblasts from human subjects with normal ATXN3 can be used as controls.

piggyBAC and Adenovirus-5 vectors are used to deliver ASREs of the disclosure to the cells, e.g., CAG8, AGC8, GCA8, CAG10, AGC10, and/or GCA10 ASREs.

Fibroblast transduction with PB-ASRE(CAG)n: Each piggyBAC (PB) ASRE(CAG)ₙ construct is co-transfected with PB transposase into the fibroblasts. Cells are transduced using DNA concentrations that favor single transposon integration events, and plated to select for puromycin resistance, which is a selectable marker present in the PB transposon. Ratios of vector to transposase are utilized to obtain 1-2 integrants per drug resistant clone. Puromycin-resistant fibroblast cell clones are picked for expansion, cryopreservation, and Western blotting to ensure the ASRE(CAG) fusion protein is expressed. ASRE⁺ (i.e., puromycin resistant) clones from the transduced experimental (mutant ATXN3) and control (normal ATXN3) fibroblasts are screened for integration of the ASRE construct. Independent clones from each group are used for ASRE expression. Each fibroblast cell clone is expanded and cultured for 72 h.

Fibroblast transduction with Ad5-ASRE(CAG)ₙ: Experimental (mutant ATXN3) and control (normal ATXN3) fibroblasts are transduced with each adenoviral-5 ASRE(CAG)ₙ construct at multiplicities of infection (MOI) ranging from 400 to 1000.

Quantification of levels of pathogenic ATXN3 RNA: The level of knockdown of the pathogenic/normal ATXN3 mRNA is assessed by RT-qPCR. Comparisons are performed 3-7 days after the fibroblasts are transduced with the Ad5-ASREs or PB-ASREs (e.g., day 3). Total RNA is isolated from the cultures, and RT-qPCR is performed to quantify the relative levels of pathogenic/normal ATXN3 in each culture condition. Each clone is analyzed in triplicate.

Quantification of levels of pathogenic ATXN3 protein: Levels of knockdown of the pathogenic and normal ATXN3 proteins are assessed by western blotting. Comparisons are performed 3-7 days after the fibroblasts are transduced with the Ad5-ASREs or PB-ASREs (e.g., day 7). Whole cell protein lysates are prepared from the cultures, and 60 mg of each sample is analyzed by western blotting. Primary antibodies are used to identify normal ATXN3 and pathogenic ATXN3, and a loading control. Blots are imaged and quantified using near-IR fluorescent secondary antibodies in a LiCor Odyssey Fc with Image Studio software. The effect of the ASREs on levels of pathogenic and normal ATXN3 protein and RNA are evaluated.

### Example 5: Treatment of Subjects with Huntington's Disease

Subjects suffering from Huntington's disease are enrolled in a clinical trial to test safety and efficacy of a site-specific RNA editing entity of the disclosure. The trial is a phase I/II, randomized, dose escalation, double-blind study.

The study includes a blinded 12-month Core Study Period to evaluate the safety and potential impact of the therapy on disease progression, and an unblinded 4-year Long-Term Period with periodic follow-up visits to evaluate safety and disease progression in treated subjects.

Subjects receive a pre-assigned dose of an adeno-associated viral (AAV) vector encoding a site-specific RNA editing entity of the disclosure (for example, 1×10\hat{ }11 genome copies (gc) to 1×10\hat{ }14 per subject depending on dose cohort), administered by MRI-guided stereotaxic infusion. Control subjects undergo a simulated surgical procedure.

Outcome measures can include number and type of Adverse Events (AE); Unified Huntington Disease Rating Scale (UHDRS) to assess changes from baseline in summary scores of domains of motor function, cognitive function, behavioral function, total functional capacity, functional independence, psychiatric symptoms and cognition; Quantitative Motor (Q-Motor) Testing to measure disease progression and responsiveness to treatment; Huntington's Disease Cognitive Assessment Battery (HD-CAB) to measure cognitive dysfunction; Magnetic Resonance Imaging (MRI) including measurements of whole brain volume, striatal region volumes, white matter volume, gray matter volume, ventricular volume, cortical thickness, basal ganglia volume, and diffusion MRI measures; Magnetic Resonance Spectroscopy (MRS) to evaluate neuronal health and gliosis; Neuro-QoL and HDQLIFE quality of life measures; Hospital Anxiety and Depression Scale (HADS); AAV vector shedding, immunogenicity response, suicidality risk [Columbia-Suicide Severity Rating Scale [C-SSRS)], and changes in global cognitive functioning [Montreal Cognitive Assessment Scale (MoCA)].

Biomarkers are evaluated, including NF-L, BDNF (Brain-derived Neurotrophic Factor), oxidative stress markers (due to mitochondrial dysfunction), and proinflammatory cytokines, in plasma and/or CSF.

### Example 6: Efficacy of (CAG)ₙ Specific ASREs for Reducing Levels of Mutant Huntingtin RNA in a Mouse Embryonic Stem Cell Model

Mouse ES cell line model of Huntington's Disease: A mouse embryonic stem (ES) cell model was utilized to evaluate efficacy of ASRE(CAG), for reducing levels of pathogenic mutant Huntingtin RNA. The cells were engineered to express one copy of Huntingtin that contains a human exon 1.

Huntington's disease is associated with a CAG trinucleotide repeat expansion that results in a longer stretch of glutamates (Q) encoded by the Htt mRNA. As a model of cells that express pathogenic mutant Huntingtin RNA and protein, mouse ES cell lines were utilized that express a copy of Huntingtin that contains a pathogenic human exon 1 sequence, with 140 copies of the CAG repeat (140 Q/Htt^{140Q}) (SEQ ID NO: 111).

As a control, a mouse ES cell line was utilized that expresses a copy of Huntingtin that contains a normal human exon 1 sequence, with 20 copies of the CAG repeat (20 Q/Htt^{20Q}) (SEQ ID NO: 112).

Both cell lines were heterozygous, with the second allele containing a mouse Huntingtin sequence including exon 1, which contains 7 CAG repeats (Htt^{140Q/7Q} and Htt^{20Q/7Q}) (SEQ ID NO: 113). The ES cell lines were generated using standard gene targeting methods.

ES cell transduction with PB-ASRE(CAG)ₙ: piggyBAC vectors were used to deliver ASREs of the disclosure to the cells. ASREs targeted against (a) CAGCAGCA (ASRE-CAG register), (b) AGCAGCAG (ASRE-AGC register), (c) GCAGCAGC (ASRE-GCA register), and (d) empty vector (VECTOR) were cloned into piggyBAC transposon vectors. Each piggyBAC ASRE(CAG)ₙ construct was co-transfected with PB transposase into the ES cells via lipofectamine transfection. Cells were transduced using DNA concentrations that favor single transposon integration events, and plated to select for puromycin resistance, which is a selectable marker present in the PB transposon. Ratios of vector to transposase were utilized to obtain 1-2 integrants per drug resistant clone. Puromycin-resistant ES cell clones were expanded to generate pools of cells that carry copies of the piggyBAC modules integrated into the genome.

Quantification of levels of pathogenic Huntingtin RNA: RNA was isolated and the levels of expression of the human 20 Q-Htt and 140 Q-Htt mRNA were assessed by RT-qPCR. Expression data were normalized to GAPDH expression within each individual sample and then compared to the VECTOR untreated experimental group to compare the level of Htt expression between experimental samples, allowing assessment of the effect of ASRE activity.

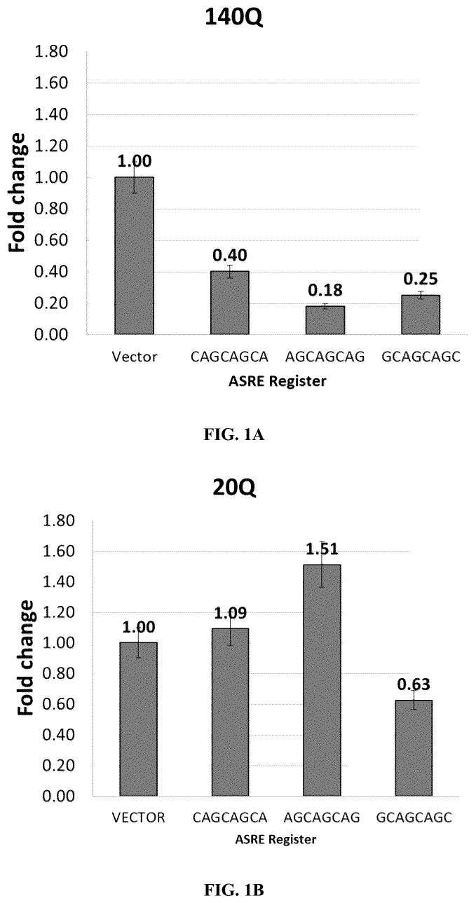

Each of the three ASREs ablated expression of pathogenic 140 Q Htt RNA (FIG. 1A), with total Htt expression reduced by approximately 60% for CAGCAGCA-targeting ASRE, 82% for AGCAGCAG-targeting ASRE, and 75% for GCAGCAGC-targeting ASRE. The effect of the ASREs on the control 20 Q Htt RNA varied, with expression most similar to the vector control for the CAGCAGCA-targeting ASRE (FIG. 1B). Maintained expression of the 20 Q Htt mRNA can be advantageous for maintaining appropriate biological function of the normal Htt allele, while specifically ablating the disease-associated RNA.

These data indicate that ASREs disclosed herein can reduce levels of target CAG repeat RNAs, including Huntington's disease-associated Htt mRNA, and can preferentially degrade pathogenic mRNAs with high CAG repeat numbers.

### Example 7: Efficacy of (CAG)ₙ Specific ASREs for Reducing Levels of Mutant Htt RNA in Primary Human Fibroblasts from a Huntington's Disease (HD) Patient

Huntington's disease is associated with pathogenic versions of Huntingtin protein (Htt) encoded by RNAs that contain a higher number of CAG repeats than RNAs that encode non-pathogenic versions of Huntingtin protein.

The impact of ASREs of the disclosure on levels of Huntingtin RNA was evaluated for primary human cells from a subject with Huntington's disease (cell line GM21756, containing one 70 Q (pathogenic) allele and one 15 Q (normal) allele).

Fibroblast transduction with Ad5-ASRE(CAG)ₙ: Adenovirus-5 vectors were used to deliver ASREs of the disclosure to the cells. ASREs were cloned into an Adenoviral Serotype 5 (RDG) shuttle vector to generate Ad5(RDG) packaged ASREs against CAGCAGCA (Ad5-ASRE-CAG), AGCAGCAG (Ad5-ASRE-AGC), and GCAGCAGC (Ad5-ASRE-GCA). The RDG modification on the Ad5 particle allows Ad5 virus to target integrin receptors that are highly expressed by skin fibroblasts. As a result, high (e.g., nearly 100%) transduction of the cells can be achieved at relatively a low multiplicity of infection (MOI).

Fibroblasts were transduced with each adenoviral-5 ASRE(CAG)ₙ construct at a MOI of 750. Untreated cells were used as a baseline Htt expression control.

Quantification of levels of pathogenic Huntingtin RNA: Five days after transduction, RNA was isolated and the level of expression of Htt mRNA was assessed by RT-qPCR. Expression data were normalized to GAPDH expression within each individual sample and then compared to the untreated group to compare the level of Htt expression between experimental samples, allowing assessment of the effect of ASRE activity in the treated groups.

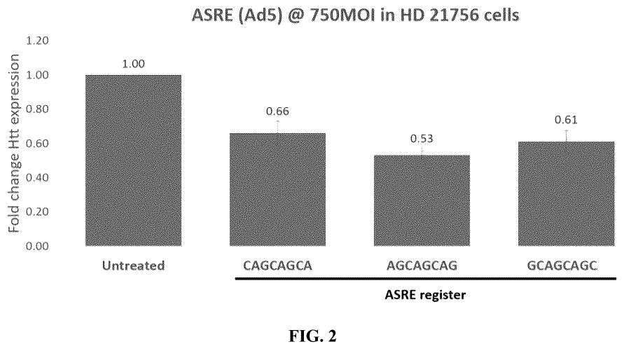

All of the ASRE candidates reduced expression of Htt, between about 34 and about 47% (FIG. 2).

These data indicate that ASREs disclosed herein can reduce levels of target CAG repeat RNAs, including Huntington's disease-associated Htt mRNA in human cells.

### Example 8: Efficacy of (CAG)ₙ Specific ASREs for Reducing Levels of Mutant Ataxin-3 RNA in Primary Human Fibroblasts from SCA3 (Machado-Joseph Disease) Patients

SCA3 is caused by a pathogenic ataxin-3 (ATXN3) protein, which can be encoded by a pathogenic RNA that comprises a higher number of CAG repeats than an RNA encoding a non-pathogenic ATXN3. Levels of ATXN3 with and without ASREs were compared in primary human fibroblasts from patients with spinocerebellar ataxia type 3 (SCA3, Machado-Joseph disease; cell references GM06153 and GM06151).

Adenovirus-5 vectors were used to deliver ASREs of the disclosure to the cells. ASREs were cloned into an Adenoviral Serotype 5 (RDG) shuttle vector to generate Ad5(RDG) packaged ASREs against CAGCAGCA (Ad5-ASRE-CAG), AGCAGCAG (Ad5-ASRE-AGC), and GCAGCAGC (Ad5-ASRE-GCA).

In a first experiment, GM06153 fibroblasts were transduced with CAGCAGCA-targeted ASRE (Ad5-ASRE-CAG) at MOI 100, 200, or 400. Untreated cells were used as a baseline expression control. Five days after transduction, RNA was isolated and the levels of expression of ATXN3 and Htt mRNA was assessed by RT-qPCR. GM06153 fibroblasts contain one 71 Q (pathogenic) ATXN3 allele and one 21 Q (normal) ATXN3 allele. The cells also contain Htt alleles of approximately 21 Q each. Htt expression was quantified as a control for activity of the ASRE on lower CAG repeat length.

Expression data were normalized to GAPDH expression within each individual sample and then compared to the untreated group to compare the levels of ATXN3 and Htt expression between experimental samples, allowing assessment of the effect of ASRE activity.

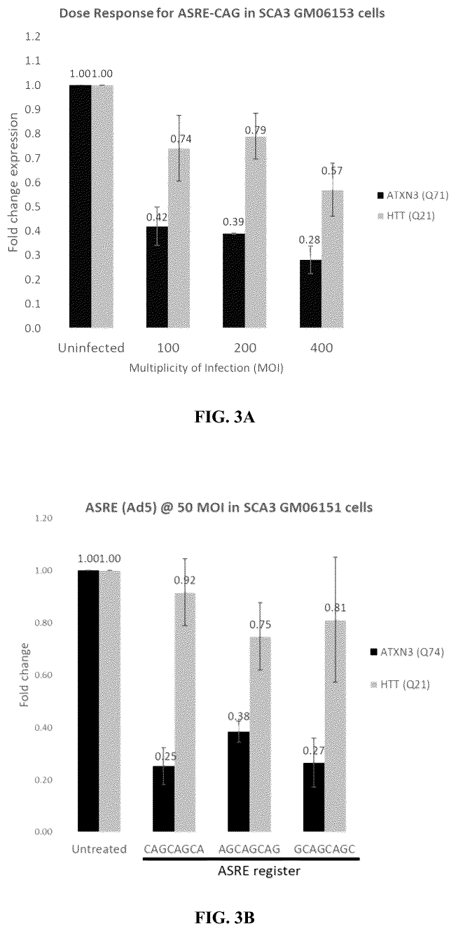

Repeat length preference was observed for all MOIs tested (FIG. 3A). ATXN3 RNA levels were reduced at approximately comparable levels at 100 and 200 MOI (˜60% reduction in expression). As the MOI was increased to 400, ˜70% reduction of ATXN3 expression was observed. At all of these MOIs, Htt expression (˜21 Q) was reduced by less than ATXN3: ˜25% at 100 and 200 MOI, and ˜45% at 400 MOI.

The expression level of ASRE assessed via qPCR was 6.6-fold higher than GAPDH at 100 MOI, 13.7-fold higher than GAPDH at 200 MOI, and 22-fold higher than GAPDH at 400 MOI. These high expression levels can be extra-physiological (e.g., compared to levels achieved by an in vivo treatment regimen disclosed herein). Despite these high levels of ASRE expression, limited “off-target” ablation of Htt mRNAs with shorter CAG repeat lengths was observed, with preferential degradation of pathogenic mRNAs with longer CAG repeats.

In a second experiment, three ASRE candidates were tested at a lower multiplicity of infection and in different patient-derived cells.

GM06151 fibroblasts were transduced with ASREs against CAGCAGCA (Ad5-ASRE-CAG), AGCAGCAG (Ad5-ASRE-AGC), or GCAGCAGC (Ad5-ASRE-GCA) at an MOI of 50. Untreated cells were used as a baseline expression control. Five days after transduction, RNA was isolated and the levels of expression of ATXN3 and Htt mRNA were assessed by RT-qPCR. GM06151 fibroblasts contain one 74 Q (pathogenic) ATXN3 allele and one 24 Q (normal) ATXN3 allele. The cells also contain Htt alleles of approximately 21 Q each. Htt expression was quantified as a control for activity of the ASRE on lower CAG repeat length.

Expression of the ASREs was lower in this experiment (MOI 50): Ad5-ASRE-CAG was expressed at approximately the same level as GAPDH; Ad5-ASRE-AGC was expressed at 0.5-fold the level of GAPDH; and Ad5-ASRE-GCA was expressed at 1.7-fold the level of GAPDH. Ablation of ATXN3 remained robust at these lower ASRE expression levels, ranging from −60-75% reduction (FIG. 3B).

For Ad5-ASRE-CAG, the level of Htt expression (˜21 Q) was only affected by approximately 8%. Maintained expression of mRNAs with lower numbers of CAG repeats (e.g., 21) can be advantageous for maintaining appropriate biological function of normal alleles, while specifically ablating disease-associated RNA with higher numbers of CAG repeats.

These data indicate that ASREs disclosed herein can reduce levels of target CAG repeat RNAs, including SCA3-associated ATXN3 mRNA, and can preferentially degrade pathogenic mRNAs with high CAG repeat numbers.

### Example 9: Treatment of Subjects with SCA3 (Machado-Joseph Disease)

Subjects suffering from SCA3 (Machado-Joseph disease) (e.g., ataxic SCA3/MJD carriers) are enrolled in a clinical trial to test safety and efficacy of a site-specific RNA editing entity of the disclosure. The trial is a phase I/II, randomized, dose escalation, double-blind study.

The study includes a blinded 12-month Core Study Period to evaluate the safety and potential impact of the therapy on disease progression, and an unblinded 4-year Long-Term Period with periodic follow-up visits to evaluate safety and disease progression in treated subjects.

Subjects receive a pre-assigned dose of an adeno-associated viral (AAV) vector encoding a site-specific RNA editing entity of the disclosure (for example, 1×10\hat{ }11 genome copies (gc) to 1×10\hat{ }14 per subject depending on dose cohort), administered by MRI-guided stereotaxic infusion. Control subjects undergo a simulated surgical procedure.

Outcome measures can include number and type of Adverse Events (AE); change in scale for the assessment and rating of ataxia (SARA) score over time; change in Composite Cerebellar Functional Severity Score (CCFS) total score over time; change in timed 25 foot walk test (T25FW) over time; change in Cerebellar Cognitive Affective Syndrome (CCAS) score over time; change in Inventory of Non-ataxia Symptoms (INAS) total count over time; change in Functional staging score (ambulatory capabilities) over time; change in cerebellar and brainstem volumes since baseline imaging; grey matter (GM) and white matter (WM) loss metrics from voxel-based morphometric (VBM) since baseline imaging; change in metabolite concentrations since baseline imaging; change in fractional isotropy since baseline imaging; change in mean diffusivity since baseline imaging; change in radial and axial diffusivity since baseline imaging; change in Friedreich's Ataxia Activities of Daily Living (FAA-ADL) over time; change in Fatigue Severity Scale (FSS) over time; change in Euro Qol-5D (EQ-5D) over time; change in Patient Health Questionnaire (PHQ-9) over time; change in Patient Global Impression (PGI) over time; changes from Baseline Spinocerebellar Ataxia Functional Index (SCAFI); changes from Baseline Wechsler Adult Intelligence Scale (WAIS—4); hanges from Baseline Structural/T1 MRI; 9-Hole Peg Board test; 8 m walking time; PATA repetition rate; Click Test; Beck Depression Inventory, Barthel Index; WHOQol; and survival.

TABLE 2
sequences

| SEQ | | |
|---|---|---|
| ID | | |
| NO: | Description | Sequence |
| 1 | PUF domain | MSVACVLKRKAVLWQDSFSPHLKHHPQEPANPNMPVVLTS |
| | | GTGSQAQPQPAANQALAAGTHSSPVPGSIGVAGRSQDDAM |
| | | VDYFFQRQHGEQLGGGGSGGGGYNNSKHRWPTGDNIHAE |
| | | HQVRSMDELNHDFQALALEGRAMGEQLLPGKKFWETDESS |
| | | KDGPKGIFLGDQWRDSAWGTSDHSVSQPIMVQRRPGQSFH |
| | | VNSEVNSVLSPRSESGGLGVSMVEYVLSSSPGDSCLRKGGF |
| | | GPRDADSDENDKGEKKNKGTFDGDKLGDLKEEGDVMDKT |
| | | NGLPVQNGIDADVKDFSRTPGNCQNSANEVDLLGPNQNGS |
| | | EGLAQLTSTNGAKPVEDFSNMESQSVPLDPMEHVGMEPLQ |
| | | FDYSGTQVPVDSAAATVGLFDYNSQQQLFQRPNALAVQQL |
| | | TAAQQQQYALAAAHQPHIGLAPAAFVPNPYIISAAPPGTDP |
| | | YTAGLAAAATLGPAVVPHQYYGVTPWGVYPASLFQQQAA |
| | | AAAAATNSANQQTTPQAQQGQQQVLRGGASQRPLTPNON |
| | | QQGQQTDPLVAAAAVNSALAFGQGLAAGMPGYPVLAPAA |
| | | YYDQTGALVVNAGARNGLGAPVRLVAPAPVIISSSAAQAA |
| | | VAAAAASANGAAGGLAGTTNGPFRPLGTQQPQPQPQQQPN |
| | | NNLASSSFYGNNSLNSNSQSSSLFSQGSAQPANTSLGFGSSSS |
| | | LGATLGSALGGFGTAVANSNTGSGSRRDSLTGSSDLYKRTS |
| | | SSLTPIGHSFYNGLSFSSSPGPVGMPLPSQGPGHSQTPPPSLSS |
| | | HGSSSSLNLGGLINGSGRYISAAPGAEAKYRSASSASSLFSP |
| | | SSTLFSSSRLRYGMSDVMPSGRSRLLEDFRNNRYPNLQLREI |
| | | AGHIMEFSQDQHGSRFIQLKLERATPAERQLVFNEILQAAYQ |
| | | LMVDVFGNYVIQKFFEFGSLEQKLALAERIRGHVLSLALOM |
| | | YGCRVIQKALEFIPSDQQNEMVRELDGHVLKCVKDQNGNH |
| | | VVQKCIECVQPQSLQFIIDAFKGQVFALSTHPYGCRVIQRILE |
| | | HCLPDQTLPILEELHQHTEQLVQDQYGNYVIQHVLEHGRPE |
| | | DKSKIVAEIRGNVLVLSQHKFASNVVEKCVTHASRTERAVL |
| | | IDEVCTMNDGPHSALYTMMKDQYANYVVQKMIDVAEPGQ |
| | | RKIVMHKIRPHIATLRKYTYGKHILAKLEKYYMKNGVDLGP |
| | | ICGPPNGII |
| | Cytosine binding | SYXXR |
| | motif | |
| | Adenine binding | CXXXQ |
| | motif | |
| | Adenine binding | SXXXQ |
| | motif | |
| | Adenine binding | NXXXQ |
| | motif | |
| | Guanine binding | SXXXE |
| | motif | |
| 6 | 10 mer PUF base | GRSRLLEDFRNNRYPNLQLREIAGHIMEFSQDQHGSRFIQLK |
| | | LERATPAERQLVFNEILQAAYQLMVDVFGNYVIQKFFEFGS |
| | | LEQKLALAERIRGHVLSLALQMYGCRVIQKALEFIPSDQQN |
| | | EMVRELDGQVFALSTHPYGCRVIQRILEHCLPDQTILEELHQ |
| | | HTEQLVQDQYGNYVIQHVLEHGRPEDKSKIVAEIRGNVLVL |
| | | SQHKFASNVVEKCVTHASRTERAVLIDEVCTALYTMMKDQ |
| | | YANYVVQKMIDVAEPGQRKIVMHKIRPHTEQLVQDQYGNY |
| | | VIQHVLEHGRPEDKSKIVAEIRGNVLVLSQHKFASNVVEKC |
| | | VTHASRTERAVLIDEVCTALYTMMKDQYANYVVQKMIDV |
| | | AEPGQRKIVMHKIRPHIATLRKYTYGKHILAKLEKYYMKNG |
| | | VDLG |
| 7 | 10 mer PUF; | GRSRLLEDFRNNRYPNLQLREIAGHIMEFSQDQHGSYFIRLK |
| | CAGCAGCAGC | LERATPAERQLVFNEILQAAYQLMVDVFGSYVIEKFFEFGSL |
| | (SEQ ID NO: 28) | EQKLALAERIRGHVLSLALQMYGCRVIQKALEFIPSDQQNE |
| | | MVRELDGQVFALSTHPYGSYVIRRILEHCLPDQTILEELHQH |
| | | TEQLVQDQYGSYVIEHVLEHGRPEDKSKIVAEIRGNVLVLS |
| | | QHKFACRVVQKCVTHASRTERAVLIDEVCTALYTMMKDQ |
| | | YASYVVRKMIDVAEPGQRKIVMHKIRPHTEQLVQDQYGSY |
| | | VIEHVLEHGRPEDKSKIVAEIRGNVLVLSQHKFACRVVQKC |
| | | VTHASRTERAVLIDEVCTALYTMMKDQYASYVVRKMIDVA |
| | | EPGQRKIVMHKIRPHIATLRKYTYGKHILAKLEKYYMKNGV |
| | | DLG |
| 8 | 10 mer PUF; | GRSRLLEDFRNNRYPNLQLREIAGHIMEFSQDQHGCRFIQLK |
| | AGCAGCAGCA | LERATPAERQLVFNEILQAAYQLMVDVFGSYVIRKFFEFGSL |
| | (SEQ ID NO: 29) | EQKLALAERIRGHVLSLALQMYGSYVIEKALEFIPSDQQNE |
| | | MVRELDGQVFALSTHPYGCRVIQRILEHCLPDQTILEELHQH |
| | | TEQLVQDQYGSYVIRHVLEHGRPEDKSKIVAEIRGNVLVLS |
| | | QHKFASYVVEKCVTHASRTERAVLIDEVCTALYTMMKDQY |
| | | ACRVVQKMIDVAEPGQRKIVMHKIRPHTEQLVQDQYGSYV |
| | | IRHVLEHGRPEDKSKIVAEIRGNVLVLSQHKFASYVVEKCV |
| | | THASRTERAVLIDEVCTALYTMMKDQYACRVVQKMIDVAE |
| | | PGQRKIVMHKIRPHIATLRKYTYGKHILAKLEKYYMKNGV |
| | | DLG |
| 9 | 10 mer PUF; | GRSRLLEDFRNNRYPNLQLREIAGHIMEFSQDQHGSYFIELK |
| | GCAGCAGCAG | LERATPAERQLVFNEILQAAYQLMVDVFGCRVIQKFFEFGS |
| | (SEQ ID NO: 30) | LEQKLALAERIRGHVLSLALQMYGSYVIRKALEFIPSDQQNE |
| | | MVRELDGQVFALSTHPYGSYVIERILEHCLPDQTILEELHQH |
| | | TEQLVQDQYGCRVIQHVLEHGRPEDKSKIVAEIRGNVLVLS |
| | | QHKFASYVVRKCVTHASRTERAVLIDEVCTALYTMMKDQY |
| | | ASYVVEKMIDVAEPGQRKIVMHKIRPHTEQLVQDQYGCRVI |
| | | QHVLEHGRPEDKSKIVAEIRGNVLVLSQHKFASYVVRKCVT |
| | | HASRTERAVLIDEVCTALYTMMKDQYASYVVEKMIDVAEP |
| | | GQRKIVMHKIRPHIATLRKYTYGKHILAKLEKYYMKNGVD |
| | | LG |
| 10 | 8 mer PUF base | GRSRLLEDFRNNRYPNLQLREIAGHIMEFSQDQHGSRFIQLK |
| | | LERATPAERQLVFNEILQAAYQLMVDVFGNYVIQKFFEFGS |
| | | LEQKLALAERIRGHVLSLALQMYGCRVIQKALEFIPSDQQN |
| | | EMVRELDGHVLKCVKDQNGNHVVQKCIECVQPEDKSKIVA |
| | | EIRGQVFALSTHPYGCRVIQRILEHCLPDQTILEELHQHTEQL |
| | | VQDQYGNYVIQHVLEHGRPEDKSKIVAEIRGNVLVLSQHKF |
| | | ASNVVEKCVTHASRTERAVLIDEVCTALYTMMKDQYANY |
| | | VVQKMIDVAEPGQRKIVMHKIRPHIATLRKYTYGKHILAKL |
| | | EKYYMKNGVDLG |
| 11 | 8 mer PUF; | GRSRLLEDFRNNRYPNLQLREIAGHIMEFSQDQHGCRFIQLK |
| | CAGCAGCA | LERATPAERQLVFNEILQAAYQLMVDVFGSYVIRKFFEFGSL |
| | | EQKLALAERIRGHVLSLALQMYGSYVIEKALEFIPSDQQNE |
| | | MVRELDGHVLKCVKDQNGNHVVQKCIECVQPEDKSKIVAE |
| | | IRGQVFALSTHPYGSYVIRRILEHCLPDQTILEELHQHTEQLV |
| | | QDQYGSYVIEHVLEHGRPEDKSKIVAEIRGNVLVLSQHKFA |
| | | NYVVQKCVTHASRTERAVLIDEVCTALYTMMKDQYASYV |
| | | VRKMIDVAEPGQRKIVMHKIRPHIATLRKYTYGKHILAKLE |
| | | KYYMKNGVDLG |
| 12 | 8 mer PUF; | GRSRLLEDFRNNRYPNLQLREIAGHIMEFSQDQHGSYFIELK |
| | AGCAGCAG | LERATPAERQLVFNEILQAAYQLMVDVFGNYVIQKFFEFGS |
| | | LEQKLALAERIRGHVLSLALQMYGSYVIRKALEFIPSDQQNE |
| | | MVRELDGHVLKCVKDQNGSYVVEKCIECVQPEDKSKIVAEI |
| | | RGQVFALSTHPYGNYVIQRILEHCLPDQTILEELHQHTEQLV |
| | | QDQYGSYVIRHVLEHGRPEDKSKIVAEIRGNVLVLSQHKFA |
| | | SYVVEKCVTHASRTERAVLIDEVCTALYTMMKDQYANYV |
| | | VQKMIDVAEPGQRKIVMHKIRPHIATLRKYTYGKHILAKLE |
| | | KYYMKNGVDLG |
| 44 | 8 mer PUF; | GRSRLLEDFRNNRYPNLQLREIAGHIMEFSQDQHGSYFIELK |
| | AGCAGCAG | LERATPAERQLVFNEILQAAYQLMVDVFGCRVIQKFFEFGS |
| | | LEQKLALAERIRGHVLSLALQMYGSYVIRKALEFIPSDQQNE |
| | | MVRELDGHVLKCVKDQNGSYVVEKCIECVQPEDKSKIVAEI |
| | | RGQVFALSTHPYGCRVIQRILEHCLPDQTILEELHQHTEQLV |
| | | QDQYGSYVIRHVLEHGRPEDKSKIVAEIRGNVLVLSQHKFA |
| | | SYVVEKCVTHASRTERAVLIDEVCTALYTMMKDQYACRVV |
| | | QKMIDVAEPGQRKIVMHKIRPHIATLRKYTYGKHILAKLEK |
| | | YYMKNGVDLG |
| 13 | 8 mer PUF; | GRSRLLEDFRNNRYPNLQLREIAGHIMEFSQDQHGSYFIRLK |
| | GCAGCAGC | LERATPAERQLVFNEILQAAYQLMVDVFGSYVIEKFFEFGSL |
| | | EQKLALAERIRGHVLSLALQMYGNYVIQKALEFIPSDQQNE |
| | | MVRELDGHVLKCVKDQNGSYVVRKCIECVQPEDKSKIVAEI |
| | | RGQVFALSTHPYGSYVIERILEHCLPDQTILEELHQHTEQLV |
| | | QDQYGNYVIQHVLEHGRPEDKSKIVAEIRGNVLVLSQHKFA |
| | | SYVVRKCVTHASRTERAVLIDEVCTALYTMMKDQYASYVV |
| | | EKMIDVAEPGQRKIVMHKIRPHIATLRKYTYGKHILAKLEK |
| | | YYMKNGVDLG |
| 14 | linker | VDTGNGS |
| | linker | VDT |
| 16 | linker | VDFVGYPRFPAPVEFI |
| 17 | linker | VDMALHARNIA |
| 18 | linker | VDLLALDREVQEL |
| 19 | linker | LLALDREVQE |
| 20 | linker | LLALDREVQ |
| 21 | linker | LLALDREV |
| 22 | linker | VDHIQRGGSP |
| 23 | linker | VDRRMARDGLVH |
| 24 | linker | FVGYPRFPAPVEFI |
| 25 | linker | LLALDREVQEL |
| 26 | linker | MALHARNIA |
| 27 | linker | LGHIQRGGSP |
| 42 | linker | VDTANGS |
| 28 | CAG repeat | CAGCAGCAGC |
| 29 | CAG repeat | AGCAGCAGCA |
| 30 | CAG repeat | GCAGCAGCAG |
| | CAG repeat | CAGCAGCA |
| | CAG repeat | AGCAGCAG |
| | CAG repeat | GCAGCAGC |
| 34 | cleavage domain | QMELEIRPLFLVPDTNGFIDHLASLARLLESRKYILVVPLIVI |
| | | NELDGLAKGQETDHRAGGYARVVQEKARKSIEFLEQRFESR |
| | | DSCLRALTSRGNELESIAFRSEDITGQLGNNADLILSCCLHYC |
| | | KDKAKDFMPASKEEPIRLLREVVLLTDDRNLRVKALTRNVP |
| | | VRDIPAFLTWAQVG |
| 35 | full length CAG8 | GRSRLLEDFRNNRYPNLQLREIAGHIMEFSQDQHGCRFIQLK |
| | ASRE | LERATPAERQLVFNEILQAAYQLMVDVFGSYVIRKFFEFGSL |
| | | EQKLALAERIRGHVLSLALQMYGSYVIEKALEFIPSDQQNE |
| | | MVRELDGHVLKCVKDQNGNHVVQKCIECVQPEDKSKIVAE |
| | | IRGQVFALSTHPYGSYVIRRILEHCLPDQTILEELHQHTEQLV |
| | | QDQYGSYVIEHVLEHGRPEDKSKIVAEIRGNVLVLSQHKFA |
| | | NYVVQKCVTHASRTERAVLIDEVCTALYTMMKDQYASYV |
| | | VRKMIDVAEPGQRKIVMHKIRPHIATLRKYTYGKHILAKLE |
| | | KYYMKNGVDLGVDTANGSQMELEIRPLFLVPDTNGFIDHL |
| | | ASLARLLESRKYILVVPLIVINELDGLAKGQETDHRAGGYA |
| | | RVVQEKARKSIEFLEQRFESRDSCLRALTSRGNELESIAFRSE |
| | | DITGQLGNNDDLILSCCLHYCKDKAKDFMPASKEEPIRLLRE |
| | | VVLLTDDRNLRVKALTRNVPVRDIPAFLTWAQV |
| 36 | full length AGC8 | GRSRLLEDFRNNRYPNLQLREIAGHIMEFSQDQHGSYFIELK |
| | ASRE | LERATPAERQLVFNEILQAAYQLMVDVFGCRVIQKFFEFGS |
| | | LEQKLALAERIRGHVLSLALQMYGSYVIRKALEFIPSDQQNE |
| | | MVRELDGHVLKCVKDQNGSYVVEKCIECVQPEDKSKIVAEI |
| | | RGQVFALSTHPYGCRVIQRILEHCLPDQTILEELHQHTEQLV |
| | | QDQYGSYVIRHVLEHGRPEDKSKIVAEIRGNVLVLSQHKFA |
| | | SYVVEKCVTHASRTERAVLIDEVCTALYTMMKDQYACRVV |
| | | QKMIDVAEPGQRKIVMHKIRPHIATLRKYTYGKHILAKLEK |
| | | YYMKNGVDLGVDTANGSQMELEIRPLFLVPDTNGFIDHLAS |
| | | LARLLESRKYILVVPLIVINELDGLAKGQETDHRAGGYARV |
| | | VQEKARKSIEFLEQRFESRDSCLRALTSRGNELESIAFRSEDI |
| | | TGQLGNNDDLILSCCLHYCKDKAKDFMPASKEEPIRLLREV |
| | | VLLTDDRNLRVKALTRNVPVRDIPAFLTWAQV |
| 37 | full length GCA8 | GRSRLLEDFRNNRYPNLQLREIAGHIMEFSQDQHGSYFIRLK |
| | ASRE | LERATPAERQLVFNEILQAAYQLMVDVFGSYVIEKFFEFGSL |
| | | EQKLALAERIRGHVLSLALQMYGNYVIQKALEFIPSDQQNE |
| | | MVRELDGHVLKCVKDQNGSYVVRKCIECVQPEDKSKIVAEI |
| | | RGQVFALSTHPYGSYVIERILEHCLPDQTILEELHQHTEQLV |
| | | QDQYGNYVIQHVLEHGRPEDKSKIVAEIRGNVLVLSQHKFA |
| | | SYVVRKCVTHASRTERAVLIDEVCTALYTMMKDQYASYVV |
| | | EKMIDVAEPGQRKIVMHKIRPHIATLRKYTYGKHILAKLEK |
| | | YYMKNGVDLGVDTANGSQMELEIRPLFLVPDTNGFIDHLAS |
| | | LARLLESRKYILVVPLIVINELDGLAKGQETDHRAGGYARV |
| | | VQEKARKSIEFLEQRFESRDSCLRALTSRGNELESIAFRSEDI |
| | | TGQLGNNDDLILSCCLHYCKDKAKDFMPASKEEPIRLLREV |
| | | VLLTDDRNLRVKALTRNVPVRDIPAFLTWAQV |
| 38 | full length CAG8 | MADYKDHEGDYKDHDIDYKDDDDKEFGRSRLLEDFRNNR |
| | ASRE with N- | YPNLQLREIAGHIMEFSQDQHGCRFIQLKLERATPAERQLVF |
| | terminal FLAG tag | NEILQAAYQLMVDVFGSYVIRKFFEFGSLEQKLALAERIRGH |
| | | VLSLALQMYGSYVIEKALEFIPSDQQNEMVRELDGHVLKCV |
| | | KDQNGNHVVQKCIECVQPEDKSKIVAEIRGQVFALSTHPYG |
| | | SYVIRRILEHCLPDQTILEELHQHTEQLVQDQYGSYVIEHVL |
| | | EHGRPEDKSKIVAEIRGNVLVLSQHKFANYVVQKCVTHASR |
| | | TERAVLIDEVCTALYTMMKDQYASYVVRKMIDVAEPGQRK |
| | | IVMHKIRPHIATLRKYTYGKHILAKLEKYYMKNGVDLGVD |
| | | TANGSQMELEIRPLFLVPDTNGFIDHLASLARLLESRKYILV |
| | | VPLIVINELDGLAKGQETDHRAGGYARVVQEKARKSIEFLE |
| | | QRFESRDSCLRALTSRGNELESIAFRSEDITGQLGNNDDLILS |
| | | CCLHYCKDKAKDFMPASKEEPIRLLREVVLLTDDRNLRVK |
| | | ALTRNVPVRDIPAFLTWAQV |
| 39 | full length AGC8 | MADYKDHEGDYKDHDIDYKDDDDKEFGRSRLLEDFRNNR |
| | ASRE with N- | YPNLQLREIAGHIMEFSQDQHGSYFIELKLERATPAERQLVF |
| | terminal FLAG tag | NEILQAAYQLMVDVFGCRVIQKFFEFGSLEQKLALAERIRG |
| | | HVLSLALQMYGSYVIRKALEFIPSDQQNEMVRELDGHVLKC |
| | | VKDQNGSYVVEKCIECVQPEDKSKIVAEIRGQVFALSTHPY |
| | | GCRVIQRILEHCLPDQTILEELHQHTEQLVQDQYGSYVIRHV |
| | | LEHGRPEDKSKIVAEIRGNVLVLSQHKFASYVVEKCVTHAS |
| | | RTERAVLIDEVCTALYTMMKDQYACRVVQKMIDVAEPGQ |
| | | RKIVMHKIRPHIATLRKYTYGKHILAKLEKYYMKNGVDLG |
| | | VDTANGSQMELEIRPLFLVPDTNGFIDHLASLARLLESRKYI |
| | | LVVPLIVINELDGLAKGQETDHRAGGYARVVQEKARKSIEF |
| | | LEQRFESRDSCLRALTSRGNELESIAFRSEDITGQLGNNDDLI |
| | | LSCCLHYCKDKAKDFMPASKEEPIRLLREVVLLTDDRNLRV |
| | | KALTRNVPVRDIPAFLTWAQV |
| 40 | full length GCA8 | MADYKDHEGDYKDHDIDYKDDDDKEFGRSRLLEDFRNNR |
| | ASRE with N- | YPNLQLREIAGHIMEFSQDQHGSYFIRLKLERATPAERQLVF |
| | terminal FLAG tag | NEILQAAYQLMVDVFGSYVIEKFFEFGSLEQKLALAERIRGH |
| | | VLSLALQMYGNYVIQKALEFIPSDQQNEMVRELDGHVLKC |
| | | VKDQNGSYVVRKCIECVQPEDKSKIVAEIRGQVFALSTHPY |
| | | GSYVIERILEHCLPDQTILEELHQHTEQLVQDQYGNYVIQHV |
| | | LEHGRPEDKSKIVAEIRGNVLVLSQHKFASYVVRKCVTHAS |
| | | RTERAVLIDEVCTALYTMMKDQYASYVVEKMIDVAEPGQR |
| | | KIVMHKIRPHIATLRKYTYGKHILAKLEKYYMKNGVDLGV |
| | | DTANGSQMELEIRPLFLVPDTNGFIDHLASLARLLESRKYIL |
| | | VVPLIVINELDGLAKGQETDHRAGGYARVVQEKARKSIEFL |
| | | EQRFESRDSCLRALTSRGNELESIAFRSEDITGQLGNNDDLIL |
| | | SCCLHYCKDKAKDFMPASKEEPIRLLREVVLLTDDRNLRVK |
| | | ALTRNVPVRDIPAFLTWAQV |
| 45 | Flag tag | MADYKDHEGDYKDHDIDYKDDDDKEF |
| 41 | cleavage domain | QMELEIRPLFLVPDTNGFIDHLASLARLLESRKYILVVPLIVI |
| | | NELDGLAKGQETDHRAGGYARVVQEKARKSIEFLEQRFESR |
| | | DSCLRALTSRGNELESIAFRSEDITGQLGNNDDLILSCCLHYC |
| | | KDKAKDFMPASKEEPIRLLREVVLLTDDRNLRVKALTRNVP |
| | | VRDIPAFLTWAQV |
| 46 | full length CAG10 | GRSRLLEDFRNNRYPNLQLREIAGHIMEFSQDQHGSYFIRLK |
| | ASRE | LERATPAERQLVFNEILQAAYQLMVDVFGSYVIEKFFEFGSL |
| | | EQKLALAERIRGHVLSLALQMYGCRVIQKALEFIPSDQQNE |
| | | MVRELDGQVFALSTHPYGSYVIRRILEHCLPDQTILEELHQH |
| | | TEQLVQDQYGSYVIEHVLEHGRPEDKSKIVAEIRGNVLVLS |
| | | QHKFACRVVQKCVTHASRTERAVLIDEVCTALYTMMKDQ |
| | | YASYVVRKMIDVAEPGQRKIVMHKIRPHTEQLVQDQYGSY |
| | | VIEHVLEHGRPEDKSKIVAEIRGNVLVLSQHKFACRVVQKC |
| | | VTHASRTERAVLIDEVCTALYTMMKDQYASYVVRKMIDVA |
| | | EPGQRKIVMHKIRPHIATLRKYTYGKHILAKLEKYYMKNGV |
| | | DLGVDTGNGSQMELEIRPLFLVPDTNGFIDHLASLARLLESR |
| | | KYILVVPLIVINELDGLAKGQETDHRAGGYARVVQEKARKS |
| | | IEFLEQRFESRDSCLRALTSRGNELESIAFRSEDITGQLGNNA |
| | | DLILSCCLHYCKDKAKDFMPASKEEPIRLLREVVLLTDDRN |
| | | LRVKALTRNVPVRDIPAFLTWAQVG |
| 47 | full length AGC10 | GRSRLLEDFRNNRYPNLQLREIAGHIMEFSQDQHGCRFIQLK |
| | ASRE | LERATPAERQLVFNEILQAAYQLMVDVFGSYVIRKFFEFGSL |
| | | EQKLALAERIRGHVLSLALQMYGSYVIEKALEFIPSDQQNE |
| | | MVRELDGQVFALSTHPYGCRVIQRILEHCLPDQTILEELHQH |
| | | TEQLVQDQYGSYVIRHVLEHGRPEDKSKIVAEIRGNVLVLS |
| | | QHKFASYVVEKCVTHASRTERAVLIDEVCTALYTMMKDQY |
| | | ACRVVQKMIDVAEPGQRKIVMHKIRPHTEQLVQDQYGSYV |
| | | IRHVLEHGRPEDKSKIVAEIRGNVLVLSQHKFASYVVEKCV |
| | | THASRTERAVLIDEVCTALYTMMKDQYACRVVQKMIDVAE |
| | | PGQRKIVMHKIRPHIATLRKYTYGKHILAKLEKYYMKNGV |
| | | DLGVDTGNGSQMELEIRPLFLVPDTNGFIDHLASLARLLESR |
| | | KYILVVPLIVINELDGLAKGQETDHRAGGYARVVQEKARKS |
| | | IEFLEQRFESRDSCLRALTSRGNELESIAFRSEDITGQLGNNA |
| | | DLILSCCLHYCKDKAKDFMPASKEEPIRLLREVVLLTDDRN |
| | | LRVKALTRNVPVRDIPAFLTWAQVG |
| 48 | Full length GCA | GRSRLLEDFRNNRYPNLQLREIAGHIMEFSQDQHGSYFIELK |
| | ASRE | LERATPAERQLVFNEILQAAYQLMVDVFGCRVIQKFFEFGS |
| | | LEQKLALAERIRGHVLSLALQMYGSYVIRKALEFIPSDQQNE |
| | | MVRELDGQVFALSTHPYGSYVIERILEHCLPDQTILEELHQH |
| | | TEQLVQDQYGCRVIQHVLEHGRPEDKSKIVAEIRGNVLVLS |
| | | QHKFASYVVRKCVTHASRTERAVLIDEVCTALYTMMKDQY |
| | | ASYVVEKMIDVAEPGQRKIVMHKIRPHTEQLVQDQYGCRVI |
| | | QHVLEHGRPEDKSKIVAEIRGNVLVLSQHKFASYVVRKCVT |
| | | HASRTERAVLIDEVCTALYTMMKDQYASYVVEKMIDVAEP |
| | | GQRKIVMHKIRPHIATLRKYTYGKHILAKLEKYYMKNGVD |
| | | LGVDTGNGSQMELEIRPLFLVPDTNGFIDHLASLARLLESRK |
| | | YILVVPLIVINELDGLAKGQETDHRAGGYARVVQEKARKSI |
| | | EFLEQRFESRDSCLRALTSRGNELESIAFRSEDITGQLGNNAD |
| | | LILSCCLHYCKDKAKDFMPASKEEPIRLLREVVLLTDDRNLR |
| | | VKALTRNVPVRDIPAFLTWAQVG |
| 49 | RNAse 1 | KESRAKKFQRQHMDSDSSPSSSSTYCNQMMRRRNMTQGLC |
| | | KPVNTFVHEPLVDVQNVCFQEKVTCKNGQGNCYKSNSSMH |
| | | ITDCRLINGSRYPNCAYRTSPKERHIIVACEGSPYVPVHFDA |
| | | SVEDST |
| 50 | RNAse 4 | QDGMYQRFLRQHVHPEETGGSDRYCDLMMQRRKMTLYHC |
| | | KRFNTFIHEDIWNIRSICSTTNIQCKNGKMNCHEGVVKVTDC |
| | | RDTGSSRAPNCRYRAIASTRRVVIACEGNPQVPVHFDG |
| 51 | RNAse 6 | WPKRLTKAHWFEIQHIQPSPLQCNRAMSGINNYTQHCKHQ |
| | | NTFLHDSFQNVAAVCDLLSIVCKNRRHNCHQSSKPVNMTD |
| | | CRLTSGKYPQCRYSAAAQYKFFIVACDPPQKSDPPYKLVPV |
| | | HLDSIL |
| 52 | RNAse 7 | APARAGFCPLLLLLLLGLWVAEIPVSAKPKGMTSSQWFKIQ |
| | | HMQPSPQACNSAMKNINKHTKRCKDLNTFLHEPFSSVAATC |
| | | QTPKIACKNGDKNCHQSHGPVSLTMCKLTSGKYPNCRYKE |
| | | KRQNKSYVVACKPPQKKDSQQFHLVPVHLDRVL |
| 53 | RNAse 8 | APARAGFCPLLLLLLLGLWVAEIPVSAKPKGMTSSQWFKIQ |
| | | HMQPSPQACNSAMKNINKHTKRCKDLNTFLHEPFSSVAATC |
| | | QTPKIACKNGDKNCHQSHGPVSLTMCKLTSGKYPNCRYKE |
| | | KRQNKSYVVACKPPQKKDSQQFHLVPVHLDRVL |
| 54 | RNAse 2 | KPPQFTWAQWFETQHINMTSQQCTNAMQVINNYQRRCKN |
| | | QNTFLLTTFANVVNVCGNPNMTCPSNKTRKNCHHSGSQVP |
| | | LIHCNLTTPSPQNISNCRYAQTPANMFYIVACDNRDQRRDPP |
| | | QYPVVPVHLDRII |
| 55 | RNAse 6PL | DKRLRDNHEWKKLIMVQHWPETVCEKIQNDCRDPPDYWTI |
| | | HGLWPDKSEGCNRSWPFNLEEIKKNWMEITDSSLPSPSMGP |
| | | APPRWMRSTPRRSTLAEAWNSTGSWTSTGGCALPPAALPSG |
| | | DLCCRPSLTAGSRGVGVDLTALHQLLHVHYSATGIIPEECSE |
| | | PTKPFQIILHHDHTEWVQSIGMPIWGTISSSESAIGKNEESQP |
| | | ACAVLSHDS |
| 56 | RNAse L | AAVEDNHLLIKAVQNEDVDLVQQLLEGGANVNFQEEEGG |
| | | WTPLHNAVQMSREDIVELLLRHGADPVLRKKNGATPFILAA |
| | | IAGSVKDLLKLFLSKGADVNECDFYGFTAFMEAAVYGKVK |
| | | ALKFLYKRGANVNLRRKTKEDQERLRKGGATALMDAAEK |
| | | GHVEVLKILLDEMGADVNACDNMGRNALIHALLSSDDSDV |
| | | EAITHLLLDHGADVNVRGERGKTPLILAVEKKHLGLVQRLL |
| | | EQEHIEINDTDSDGKTALLLAVELKLKKIAELLCKRGASTDC |
| | | GDLVMTARRNYDHSLVKVLLSHGAKEDFHPPAEDWKPQSS |
| | | HWGAALKDLHRIYRPMIGKLKFFIDEKYKIADTSEGGIYLGF |
| | | YEKQEVAVKTFCEGSPRAQREVSCLQSSRENSHLVTFYGSE |
| | | SHRGHLFVCVTLCEQTLEACLDVHRGEDVENEEDEFARNV |
| | | LSSIFKAVQELHLSCGYTHQDLQPQNILIDSKKAAHLADFDK |
| | | SIKWAGDPQEVKRDLEDLGRLVLYVVKKGSISFEDLKAQSN |
| | | EEVVQLSPDEETKDLIHRLFHPGEHVRDCLSDLLGHPFFWT |
| | | WESRYRTLRNVGNESDIKTRKSESEILRLLQPGPSEHSKSFD |
| | | KWTTKINECVMKKMNKFYEKRGNFYQNTVGDLLKFIRNLG |
| | | EHIDEEKHKKMKLKIGDPSLYFQKTFPDLVIYVYTKLQNTE |
| | | YRKHFPQTHSPNKPQCDGAGGASGLASPGC |
| 57 | RNAse T2 | VQHWPETVCEKIQNDCRDPPDYWTIHGLWPDKSEGCNRSW |
| | | PFNLEEIKDLLPEMRAYWPDVIHSFPNRSRFWKHEWEKHGT |
| | | CAAQVDALNSQKKYFGRSLELYRELDLNSVLLKLGIKPSIN |
| | | YYQVADFKDALARVYGVIPKIQCLPPSQDEEVQTIGQIELCL |
| | | TKQDQQLQNCTEPGEQPSPKQEVWLANGAAESRGLRVCED |
| | | GPVFYPPPKKTKH |
| 58 | RNAse 11 | EASESTMKIIKEEFTDEEMQYDMAKSGQEKQTIEILMNPILL |
| | | VKNTSLSMSKDDMSSTLLTFRSLHYNDPKGNSSGNDKECC |
| | | NDMTVWRKVSEANGSCKWSNNFIRSSTEVMRRVHRAPSCK |
| | | FVQNPGISCCESLELENTVCQFTTGKQFPRCQYHSVTSLEKIL |
| | | TVLTGHSLMSWLVCGSKL |
| 59 | RNAse T2 like | XLGGADKRLRDNHEWKKLIMVQHWPETVCEKIQNDCRDPP |
| | | DYWTIHGLWPDKSEGCNRSWPFNLEEIKDLLPEMRAYWPD |
| | | VIHSFPNRSRFWKHEWEKHGTCAAQVDALNSQKKYFGRSL |
| | | ELYRELDLNSVLLKLGIKPSINYYQTTEEDLNLDVEPTTEDT |
| | | AEEVTIHVLLHSALFGEIGPRRW |
| 60 | RNAse1 K41R | KESRAKKFQRQHMDSDSSPSSSSTYCNQMMRRRNMTQGRC |
| | | RPVNTFVHEPLVDVQNVCFQEKVTCKNGQGNCYKSNSSMH |
| | | ITDCRLINGSRYPNCAYRTSPKERHIIVACEGSPYVPVHFDA |
| | | SVEDST |
| 61 | Rnase1 (K41R, | KESRAKKFQRQHMDSDSSPSSSSTYCNQMMRRRNMTQGRC |
| | D121E) | RPVNTFVHEPLVDVQNVCFQEKVTCKNGQGNCYKSNSSMH |
| | | ITDCRLTNGSRYPNCAYRTSPKERHIIVACEGSPYVPVHFEA |
| | | SVEDST |
| 62 | Rnase1 (K41R, | KESRAKKFQRQHMDSDSSPSSSSTYCNQMMRRRNMTQGRC |
| | D121E, H119N) | RPVNTFVHEPLVDVQNVCFQEKVTCKNGQGNCYKSNSSMH |
| | | ITDCRLINGSRYPNCAYRTSPKERHIIVACEGSPYVPVNFEA |
| | | SVEDST |
| 63 | Rnase1 (H119N) | KESRAKKFQRQHMDSDSSPSSSSTYCNQMMRRRNMTQGRC |
| | | KPVNTFVHEPLVDVQNVCFQEKVTCKNGQGNCYKSNSSMH |
| | | ITDCRLINGSRYPNCAYRTSPKERHIIVACEGSPYVPVNFDA |
| | | SVEDST |
| 64 | Rnase1 (R39D, | KESRAKKFQRQHMDSDSSPSSSSTYCNQMMRRRNMTQGDC |
| | N67D, N88A, | KPVNTFVHEPLVDVQNVCFQEKVTCKDGQGNCYKSNSSMH |
| | G89D, R91D, | ITDCRLTADSDYPNCAYRTSPKERHIIVACEGSPYVPVNFDA |
| | H119N) | SVEDST |
| 65 | RNAse1 (R39D, | KESRAKKFQRQHMDSDSSPSSSSTYCNQMMRRRNMTQGDC |
| | N67D, N88A, | RPVNTFVHEPLVDVQNVCFQEKVTCKDGQGNCYKSNSSMH |
| | G89D, R91D, | ITDCRLTADSDYPNCAYRTSPKERHIIVACEGSPYVPVNFEA |
| | H119N, K41R, | SVEDST |
| | D121E) | |
| 66 | Rnase1 (R39D, | KESRAKKFQRQHMDSDSSPSSSSTYCNQMMRRRNMTQGDC |
| | N67D, N88A, | KPVNTFVHEPLVDVQNVCFQEKVTCKDGQGNCYKSNSSMH |
| | G89D, R91D) | ITDCRLTADSDYPNCAYRTSPKERHIIVACEGSPYVPVHFDA |
| | | SVEDST |
| 67 | (Rnase1 (R39D, | KESRAKKFQRQHMDSDSSPSSSSTYCNQMMRRRNMTQGDC |
| | N67D, N88A, | RPVNTFVHEPLVDVQNVCFQEKVTCKDGQGNCYKSNSSMH |
| | G89D, R91D, | ITDCRLTADSDYPNCAYRTSPKERHIIVACEGSPYVPVNFEA |
| | H119N, K41R, | SVEDST |
| | D121E) | |
| 68 | NOB1 | APVEHVVADAGAFLRHAALQDIGKNIYTIREVVTEIRDKAT |
| | | RRRLAVLPYELRFKEPLPEYVRLVTEFSKKTGDYPSLSATDI |
| | | QVLALTYQLEAEFVGVSHLKQEPQKVKVSSSIQHPETPLHIS |
| | | GFHLPYKPKPPQETEKGHSACEPENLEFSSFMFWRNPLPNID |
| | | HELQELLIDRGEDVPSEEEEEEENGFEDRKDDSDDDGGGWI |
| | | TPSNIKQIQQELEQCDVPEDVRVGCLTTDFAMQNVLLQMGL |
| | | HVLAVNGMLIREARSYILRCHGCFKTTSDMSRVFCSHCGNK |
| | | TLKKVSVTV |
| 69 | ENDOV | AFSGLQRVGGVDVSFVKGDSVRACASLVVLSFPELEVVYEE |
| | | SRMVSLTAPYVSGFLAFREVPFLLELVQQLREKEPGLMPQV |
| | | LLVDGNGVLHHRGFGVACHLGVLTDLPCVGVAKKLLQVD |
| | | GLENNALHKEKIRLLQTRGDSFPLLGDSGTVLGMALRSHDR |
| | | STRPLYISVGHRMSLEAAVRLTCCCCRFRIPEPVRQADICSRE |
| | | HIRKS |
| 70 | ENDOG | AELPPVPGGPRGPGELAKYGLPGLAQLKSRESYVLCYDPRT |
| | | RGALWVVEQLRPERLRGDGDRRECDFREDDSVHAYHRAT |
| | | NADYRGSGFDRGHLAAAANHRWSQKAMDDTFYLSNVAPQ |
| | | VPHLNQNAWNNLEKYSRSLTRSYQNVYVCTGPLFLPRTEA |
| | | DGKSYVKYQVIGKNHVAVPTHFFKVLILEAAGGQIELRTYV |
| | | MPNAPVDEAIPLERFLVPIESIERASGLLFVPNILARAGSLKAI |
| | | TAGSK |
| 71 | ENDOD1 | RLVGEEEAGFGECDKFFYAGTPPAGLAADSHVKICQRAEGA |
| | | ERFATLYSTRDRIPVYSAFRAPRPAPGGAEQRWLVEPQIDDP |
| | | NSNLEEAINEAEAITSVNSLGSKQALNTDYLDSDYQRGQLY |
| | | PFSLSSDVQVATFTLTNSAPMTQSFQERWYVNLHSLMDRAL |
| | | TPQCGSGEDLYILTGTVPSDYRVKDKVAVPEFVWLAACCA |
| | | VPGGGWAMGFVKHTRDSDIIEDVMVKDLQKLLPFNPQLFQ |
| | | NNCGETEQDTEKMKKILEVVNQIQDEERMVQSQKSSSPLSS |
| | | TRSKRSTLLPPEASEGSSSFLGKLMGFIATPFIKLFQLIYYLVV |
| | | AILKNIVYFLWCVTKQVINGIESCLYRLGSATISYFMAIGEEL |
| | | VSIPWKVLKVVAKVIRALLRILCCLLKAICRVLSIPVRVLVD |
| | | VATFPVYTMGAIPIVCKDIALGLGGTVSLLFDTAFGTLGGLF |
| | | QVVFSVCKRIGYKVTFDNSGEL |
| 72 | hFEN1 | MGIQGLAKLIADVAPSAIRENDIKSYFGRKVAIDASMSIYQF |
| | | LIAVRQGGDVLQNEEGETTSHLMGMFYRTIRMMENGIKPV |
| | | YVFDGKPPQLKSGELAKRSERRAEAEKQLQQAQAAGAEQE |
| | | VEKFTKRLVKVTKQHNDECKHLLSLMGIPYLDAPSEAEASC |
| | | AALVKAGKVYAAATEDMDCLTFGSPVLMRHLTASEAKKLP |
| | | IQEFHLSRILQELGLNQEQFVDLCILLGSDYCESIRGIGPKRA |
| | | VDLIQKHKSIEEIVRRLDPNKYPVPENWLHKEAHQLFLEPEV |
| | | LDPESVELKWSEPNEEELIKFMCGEKQFSEERIRSGVKRLSK |
| | | SRQGSTQGRLDDFFKVTGSLSSAKRKEPEPKGSTKKKAKTG |
| | | AAGKFKRGK |
| 73 | ERCC4 | MESGQPARRIAMAPLLEYERQLVLELLDTDGLVVCARGLG |
| | | ADRLLYHFLQLHCHPACLVLVLNTQPAEEEYFINQLKIEGVE |
| | | HLPRRVTNEITSNSRYEVYTQGGVIFATSRILVVDFLTDRIPS |
| | | DLITGILVYRAHRIIESCQEAFILRLFRQKNKRGFIKAFTDNA |
| | | VAFDTGFCHVERVMRNLFVRKLYLWPRFHVAVNSFLEQHK |
| | | PEVVEIHVSMTPTMLAIQTAILDILNACLKELKCHNPSLEVE |
| | | DLSLENAIGKPFDKTIRHYLDPLWHQLGAKTKSLVQDLKIL |
| | | RTLLQYLSQYDCVTFLNLLESLRATEKAFGQNSGWLFLDSS |
| | | TSMFINARARVYHLPDAKMSKKEKISEKMEIKEGEGILWG |
| 74 | NTHL | CSPQESGMTALSARMLTRSRSLGPGAGPRGCREEPGPLRRR |
| | | EAAAEARKSHSPVKRPRKAQRLRVAYEGSDSEKGEGAEPL |
| | | KVPVWEPQDWQQQLVNIRAMRNKKDAPVDHLGTEHCYDS |
| | | SAPPKVRRYQVLLSLMLSSQTKDQVTAGAMQRLRARGLTV |
| | | DSILQTDDATLGKLIYPVGFWRSKVKYIKQTSAILQQHYGG |
| | | DIPASVAELVALPGVGPKMAHLAMAVAWGTVSGIAVDTHV |
| | | HRIANRLRWTKKATKSPEETRAALEEWLPRELWHEINGLLV |
| | | GFGQQTCLPVHPRCHACLNQALCPAAQGL |
| 75 | hSLFN14 | ESTHVEFKRFTTKKVIPRIKEMLPHYVSAFANTQGGYVLIGV |
| | | DDKSKEVVGCKWEKVNPDLLKKEIENCIEKLPTFHFCCEKP |
| | | KVNFTTKILNVYQKDVLDGYVCVIQVEPFCCVVFAEAPDS |
| | | WIMKDNSVTRLTAEQWVVMMLDTQSAPPSLVTDYNSCLIS |
| | | SASSARKSPGYPIKVHKFKEALQ |
| 76 | hLACTB2 | TLQGTNTYLVGTGPRRILIDTGEPAIPEYISCLKQALTEFNTAI |
| | | QEIVVTHWHRDHSGGIGDICKSINNDTTYCIKKLPRNPQREE |
| | | IIGNGEQQYVYLKDGDVIKTEGATLRVLYTPGHTDDHMALL |
| | | LEEENAIFSGDCILGEGTTVFEDLYDYMNSLKELLKIKADIIY |
| | | PGHGPVIHNAEAKIQQYISHRNIREQQILTLFRENFEKSFTVM |
| | | ELVKIIYKNTPENLHEMAKHNLLLHLKKLEKEGKIFSNTDPD |
| | | KKWKAHL |
| 77 | APEX2 | MLRVVSWNINGIRRPLQGVANQEPSNCAAVAVGRILDELD |
| | | ADIVCLQETKVTRDALTEPLAIVEGYNSYFSFSRNRSGYSGV |
| | | ATFCKDNATPVAAEEGLSGLFATQNGDVGCYGNMDEFTQE |
| | | ELRALDSEGRALLTQHKIRTWEGKEKTLTLINVYCPHADPG |
| | | RPERLVFKMRFYRLLQIRAEALLAAGSHVIILGDLNTAHRPI |
| | | DHWDAVNLECFEEDPGRKWMDSLLSNLGCQSASHVGPFID |
| | | SYRCFQPKQEGAFTCWSAVTGARHLNYGSRLDYVLGDRTL |
| | | VIDTFQASFLLPEVMGSDHCPVGAVLSVSSVPAKQCPPLCTR |
| | | FLPEFAGTQLKILRFLVPLEQSPVLEQSTLOHNNQTRVQTCQ |
| | | NKAQVRSTRPQPSQVGSSRGQKNLKSYFQPSPSCPQASPDIE |
| | | LPSLPLMSALMTPKTPEEKAVAKVVKGQAKTSEAKDEKEL |
| | | RTSFWKSVLAGPLRTPLCGGHREPCVMRTVKKPGPNLGRRF |
| | | YMCARPRGPPTDPSSRCNFFLWSRPS |
| 78 | APEX2 | MLRVVSWNINGIRRPLQGVANQEPSNCAAVAVGRILDELD |
| | | ADIVCLQETKVTRDALTEPLAIVEGYNSYFSFSRNRSGYSGV |
| | | ATFCKDNATPVAAEEGLSGLFATQNGDVGCYGNMDEFTQE |
| | | ELRALDSEGRALLTQHKIRTWEGKEKTLTLINVYCPHADPG |
| | | RPERLVFKMRFYRLLQIRAEALLAAGSHVIILGDLNTAHRPI |
| | | DHWDAVNLECFEEDPGRKWMDSLLSNLGCQSASHVGPFID |
| | | SYRCFQPKQEGAFTCWSAVTGARHLNYGSRLDYVLGDRTL |
| | | VIDTFQASFLLPEVMGSDHCPVGAVLSVSSVPAKQCPPLCTR |
| | | FLPEFAGTQLKILRFLVPLEQSP |
| 79 | ANG | QDNSRYTHFLTQHYDAKPQGRDDRYCESIMRRRGLTSPCK |
| | | DINTFIHGNKRSIKAICENKNGNPHRENLRISKSSFQVTTCKL |
| | | HGGSPWPPCQYRATAGFRNVVVACENGLPVHLDQSIFRRP |
| 80 | HRSP12 | SSLIRRVISTAKAPGAIGPYSQAVLVDRTIYISGQIGMDPSSG |
| | | QLVSGGVAEEAKQALKNMGEILKAAGCDFTNVVKTTVLLA |
| | | DINDFNTVNEIYKQYFKSNFPARAAYQVAALPKGSRIEIEAV |
| | | AIQGPLTTASL |
| 81 | ZC3H12A | GGGTPKAPNLEPPLPEEEKEGSDLRPVVIDGSNVAMSHGNK |
| | | EVFSCRGILLAVNWFLERGHTDITVFVPSWRKEQPRPDVPIT |
| | | DQHILRELEKKKILVFTPSRRVGGKRVVCYDDRFIVKLAYES |
| | | DGIVVSNDTYRDLQGERQEWKRFIEERLLMYSFVNDKFMPP |
| | | DDPLGRHGPSLDNFLRKKPLTLE |
| 82 | ZC3H12A | SGPCGEKPVLEASPTMSLWEFEDSHSRQGTPRPGQELAAEE |
| | | ASALELQMKVDFFRKLGYSSTEIHSVLQKLGVQADTNTVLG |
| | | ELVKHGTATERERQTSPDPCPQLPLVPRGGGTPKAPNLEPPL |
| | | PEEEKEGSDLRPVVIDGSNVAMSHGNKEVFSCRGILLAVNW |
| | | FLERGHTDITVFVPSWRKEQPRPDVPITDQHILRELEKKKILV |
| | | FTPSRRVGGKRVVCYDDRFIVKLAYESDGIVVSNDTYRDLQ |
| | | GERQEWKRFIEERLLMYSFVNDKFMPPDDPLGRHGPSLDNF |
| | | LRKKPLTLEHRKQPCPYGRKCTYGIKCRFFHPERPSCPQRSV |
| | | ADELRANALLSPPRAPSKDKNGRRPSPSSQSSSLLTESEQCSL |
| | | DGKKLGAQASPGSRQEGLTQTYAPSGRSLAPSGGSGSSFGP |
| | | TDWLPQTLDSLPYVSQDCLDSGIGSLESQMSELWGVRGGGP |
| | | GEPGPPRAPYTGYSPYGSELPATAAFSAFGRAMGAGHFSVP |
| | | ADYPPAPPAFPPREYWSEPYPLPPPTSVLQEPPVQSPGAGRSP |
| | | WGRAGSLAKEQASVYTKLCGVFPPHLVEAVMGRFPQLLDP |
| | | QQLAAEILSYKSQHPSE |
| 83 | APEX1 | PKRGKKGAVAEDGDELRTEPEAKKSKTAAKKNDKEAAGE |
| | | GPALYEDPPDQKTSPSGKPATLKICSWNVDGLRAWIKKKGL |
| | | DWVKEEAPDILCLQETKCSENKLPAELQELPGLSHQYWSAP |
| | | SDKEGYSGVGLLSRQCPLKVSYGIGDEEHDQEGRVIVAEFD |
| | | SFVLVTAYVPNAGRGLVRLEYRQRWDEAFRKFLKGLASRK |
| | | PLVLCGDLNVAHEEIDLRNPKGNKKNAGFTPQERQGFGELL |
| | | QAVPLADSFRHLYPNTPYAYTFWTYMMNARSKNVGWRLD |
| | | YFLLS |
| 84 | PDL6 | EALFFPSQVTCTEALLRAPGAELAELPEGCPCGLPHGESALS |
| | | RLLRALLAARASLDLCLFAFSSPQLGRAVQLLHQRGVRVRV |
| | | VTDCDYMALNGSQIGLLRKAGIQVRHDQDPGYMHHKFAIV |
| | | DKRVLITGSLNWTTQAIQNNRENVLITEDDEYVRLFLEEFER |
| | | IWEQFNPTKYTFFPPKKSHGSCAPPVSRAGGRLLSWHRTCG |
| | | TSSESQT |
| 85 | KIAA0391 | KARYKTLEPRGYSLLIRGLIHSDRWREALLLLEDIKKVITPSK |
| | | KNYNDCIQGALLHQDVNTAWNLYQELLGHDIVPMLETLKA |
| | | FFDFGKDIKDDNYSNKLLDILSYLRNNQLYPGESFAHSIKTW |
| | | FESVPGKQWKGQFTTVRKSGQCSGCGKTIESIQLSPEEYECL |
| | | KGKIMRDVIDGGDQYRKTTPQELKRFENFIKSRPPFDVVIDG |
| | | LNVAKMFPKVRESQLLLNVVSQLAKRNLRLLVLGRKHMLR |
| | | RSSQWSRDEMEEVQKQASCFFADDISEDDPFLLYATLHSGN |
| | | HCRFITRDLMRDHKACLPDAKTQRLFFKWQQGHQLAIVNR |
| | | FPGSKLTFQRILSYDTVVQTTGDSWHIPYDEDLVERCSCEVP |
| | | TKWLCLHQKT |
| 86 | AGO2 | SVEPMFRHLKNTYAGLQLVVVILPGKTPVYAEVKRVGDTV |
| | | LGMATQCVQMKNVQRTTPQTLSNLCLKINVKLGGVNNILL |
| | | PQGRPPVFQQPVIFLGADVTHPPAGDGKKPSIAAVVGSMDA |
| | | HPNRYCATVRVQQHRQEIIQDLAAMVRELLIQFYKSTRFKP |
| | | TRIIFYRDGVSEGQFQQVLHHELLAIREACIKLEKDYQPGITF |
| | | IVVQKRHHTRLFCTDKNERVGKSGNIPAGTTVDTKITHPTEF |
| | | DFYLCSHAGIQGTSRPSHYHVLWDDNRFSSDELQILTYQLC |
| | | HTYVRCTRSVSIPAPAYYAHLVAFRARYHLVDKEHDSAEGS |
| | | HTSGQSNGRDHQALAKAVQVHQDTLRTMYFA |
| 87 | EXOG | QGAEGALTGKQPDGSAEKAVLEQFGFPLTGTEARCYTNHA |
| | | LSYDQAKRVPRWVLEHISKSKIMGDADRKHCKFKPDPNIPP |
| | | TFSAFNEDYVGSGWSRGHMAPAGNNKFSSKAMAETFYLSN |
| | | IVPQDFDNNSGYWNRIEMYCRELTERFEDVWVVSGPLTLPQ |
| | | TRGDGKKIVSYQVIGEDNVAVPSHLYKVILARRSSVSTEPLA |
| | | LGAFVVPNEAIGFQPQLTEFQVSLQDLEKLSGLVFFPHLDRT |
| | | SDIRNICSVDTCKLLDFQEFTLYLSTRKIEGARSVLRLEKIME |
| | | NLKNAEIEPDDYFMSRYEKKLEELKAKEQSGTQIRKPS |
| 88 | ZC3H12D | EHPSKMEFFQKLGYDREDVLRVLGKLGEGALVNDVLQELI |
| | | RTGSRPGALEHPAAPRLVPRGSCGVPDSAQRGPGTALEEDF |
| | | RTLASSLRPIVIDGSNVAMSHGNKETFSCRGIKLAVDWFRD |
| | | RGHTYIKVFVPSWRKDPPRADTPIREQHVLAELERQAVLVY |
| | | TPSRKVHGKRLVCYDDRYIVKVAYEQDGVIVSNDNYRDLQ |
| | | SENPEWKWFIEQRLLMFSFVNDRFMPPDDPLGRHGPSLSNF |
| | | LSRKPKPPEPSWQHCPYGKKCTYGIKCKFYHPERPHHAQLA |
| | | VADELRAKTGARPGAGAEEQRPPRAPGGSAGARAAPREPF |
| | | AHSLPPARGSPDLAALRGSFSRLAFSDDLGPLGPPLPVPACS |
| | | LTPRLGGPDWVSAGGRVPGPLSLPSPESQFSPGDLPPPPGLQ |
| | | LQPRGEHRPRDLHGDLLSPRRPPDDPWARPPRSDRFPGRSV |
| | | WAEPAWGDGATGGLSVYATEDDEGDARARARIALYSVFPR |
| | | DQVDRVMAAFPELSDLARLILLVQRCQSAGAPLGKP |
| 89 | ERN2 | RQQQPQVVEKQQETPLAPADFAHISQDAQSLHSGASRRSQK |
| | | RLQSPSKQAQPLDDPEAEQLTVVGKISFNPKDVLGRGAGGT |
| | | FVFRGQFEGRAVAVKRLLRECFGLVRREVQLLQESDRHPNV |
| | | LRYFCTERGPQFHYIALELCRASLQEYVENPDLDRGGLEPEV |
| | | VLQQLMSGLAHLHSLHIVHRDLKPGNILITGPDSQGLGRVV |
| | | LSDFGLCKKLPAGRCSFSLHSGIPGTEGWMAPELLQLLPPDS |
| | | PTSAVDIFSAGCVFYYVLSGGSHPFGDSLYRQANILTGAPCL |
| | | AHLEEEVHDKVVARDLVGAMLSPLPQPRPSAPQVLAHPFF |
| | | WSRAKQLQFFQDVSDWLEKESEQEPLVRALEAGGCAVVRD |
| | | NWHEHISMPLQTDLRKFRSYKGTSVRDLLRAVRNKKHHYR |
| | | ELPVEVRQALGQVPDGFVQYFTNRFPRLLLHTHRAMRSCAS |
| | | ESLFLPYYPPDSEARRPCPGATGR |
| 90 | PELO | KLVRKNIEKDNAGQVTLVPEEPEDMWHTYNLVQVGDSLRA |
| | | STIRKVOTESSTGSVGSNRVRTTLTLCVEAIDFDSQACQLRV |
| | | KGTNIQENEYVKMGAYHTIELEPNRQFTLAKKQWDSVVLE |
| | | RIEQACDPAWSADVAAVVMQEGLAHICLVTPSMTLTRAKV |
| | | EVNIPRKRKGNCSQHDRALERFYEQVVQAIQRHIHFDVVKC |
| | | ILVASPGFVREQFCDYLFQQAVKTDNKLLLENRSKFLQVHA |
| | | SSGHKYSLKEALCDPTVASRLSDTKAAGEVKALDDFYKML |
| | | QHEPDRAFYGLKQVEKANEAMAIDTLLISDELFRHQDVATR |
| | | SRYVRLVDSVKENAGTVRIFSSLHVSGEQLSQLTGVAAILRF |
| | | PVPELSDQEGDSSSEED |
| 91 | YBEY | SLVIRNLQRVIPIRRAPLRSKIEIVRRILGVQKFDLGIICVDNK |
| | | NIQHINRIYRDRNVPTDVLSFPFHEHLKAGEFPQPDFPDDYN |
| | | LGDIFLGVEYIFHQCKENEDYNDVLTVTATHGLCHLLGFTH |
| | | GTEAEWQQMFQKEKAVLDELGRRTGTRLQPLTRGLFGGS |
| 92 | CPSF4L | QEVIAGLERFTFAFEKDVEMQKGTGLLPFQGMDKSASAVC |
| | | NFFTKGLCEKGKLCPFRHDRGEKMVVCKHWLRGLCKKGD |
| | | HCKFLHQYDLTRMPECYFYSKFGDCSNKECSFLHVKPAFKS |
| | | QDCPWYDQGFCKDGPLCKYRHVPRIMCLNYLVGFCPEGPK |
| | | CQFAQKIREFKLLPGSKI |
| 93 | hCG 2002731 | KLVRKNIEKDNAGQVTLVPEEPEDMWHTYNLVQVGDSLRA |
| | | STIRKVOTESSTGSVGSNRVRTTLTLCVEAIDFDSQACQLRV |
| | | KGTNIQENEYVKMGAYHTIELEPNRQFTLAKKQWDSVVLE |
| | | RIEQACDPAWSADVAAVVMQEGLAHICLVTPSMTLTRAKV |
| | | EVNIPRKRKGNCSQHDRALERFYEQVVQAIQRHIHFDVVKC |
| | | ILVASPGFVREQFCDYMFQQAVKTDNKLLLENRSKFLQVHA |
| | | SSGHKYSLKEALCDPTVASRLSDTKAAGEVKALDDFYKML |
| | | QHEPDRAFYGLKQVEKANEAMAIDTLLISDELFRHQDVATR |
| | | SRYVRLVDSVKENAGTVRIFSSLHVSGEQLSQLTGVAAILRF |
| | | PVPELSDQEGDSSSEED |
| 94 | hCG 2002731 | DPAWSADVAAVVMQEGLAHICLVTPSMTLTRAKVEVNIPR |
| | | KRKGNCSQHDRALERFYEQVVQAIQRHIHFDVVKCILVASP |
| | | GFVREQFCDYMFQQAVKTDNKLLLENRSKFLQVHASSGHK |
| | | YSLKEALCDPTVASRLSDTKAAGEVKALDDFYKMLQHEPD |
| | | RAFYGLKQVEKANEAMAIDTLLISDELFRHQDVATRSRYVR |
| | | LVDSVKENAGTVRIFSSLHVSGEQLSQLTGVAAILRFPVPEL |
| | | SDQEGDSSSEED |
| 95 | ERCC1 | MDPGKDKEGVPQPSGPPARKKFVIPLDEDEVPPGVRGNPVL |
| | | KFVRNVPWEFGDVIPDYVLGQSTCALFLSLRYHNLHPDYIH |
| | | GRLQSLGKNFALRVLLVQVDVKDPQQALKELAKMCILADC |
| | | TLILAWSPEEAGRYLETYKAYEQKPADLLMEKLEQDFVSRV |
| | | TECLTTVKSVNKTDSQTLLTTFGSLEQLIAASREDLALCPGL |
| | | GPQK |
| 96 | RAC1 | KESRAKKFQRQHMDSDSSPSSSSTYCNQMMRRRNMTQGRC |
| | | KPVNTFVHEPLVDVQNVCFQEKVTCKNGQGNCYKSNSSMH |
| | | ITDCRLINGSRYPNCAYRTSPKERHIIVACEGSPYVPVHFDA |
| | | SVEDST |
| 97 | RAA1 | QDNSRYTHFLTQHYDAKPQGRDDRYCESIMRRRGLTSPCK |
| | | DINTFIHGNKRSIKAICENKNGNPHRENLRISKSSFQVTTCKL |
| | | HGGSPWPPCQYRATAGFRNVVVACENGLPVHLDQSIFRRP |
| 98 | RAB1 | GLGLVQPSYGQDGMYQRFLRQHVHPEETGGSDRYCNLMM |
| | | QRRKMTLYHCKRFNTFIHEDIWNIRSICSTTNIQCKNGKMNC |
| | | HEGVVKVTDCRDTGSSRAPNCRYRAIASTRRVVIACEGNPQ |
| | | VPVHFDG |
| 99 | DNA2 | XSAVDNILLKLAKFKIGFLRLGQIQKVHPAIQQFTEQEICRSK |
| | | SIKSLALLEELYNSQLIVATTCMGINHPIFSRKIFDFCIVDEAS |
| | | QISQPICLGPLFFSRRFVLVGDHQQLPPLVLNREARALGMSE |
| | | SLFKRLEQNKSAVVQLTVQYRMNSKIMSLSNKLTYEGKLEC |
| | | GSDKVANAVINLRHFKDVKLELEFYADYSDNPWLMGVFEP |
| | | NNPVCFLNTDKVPAPEQVEKGGVSNVTEAKLIVFLTSIFVKA |
| | | GCSPSDIGIIAPYRQQLKIINDLLARSIGMVEVNTVDKYQGR |
| | | DKSIVLVSFVRSNKDGTVGELLKDWRRLNVAITRAKHKLIL |
| | | LGCVPSLNCYPPLEKLLNHLNSEKLISFFFCIWSHLIALL |
| 100 | FLJ35220 | MALRSHDRSTRPLYISVGHRMSLEAAVRLTCCCCRFRIPEPV |
| | | RQADICSREHIRKSLGLPGPPTPRSPKAQRPVACPKGDSGESS |
| | | ALC |
| 101 | FLJ13173 | CYTNHALSYDQAKRVPRWVLEHISKSKIMGDADRKHCKFK |
| | | PDPNIPPTFSAFNEDYVGSGWSRGHMAPAGNNKFSSKAMA |
| | | ETFYLSNIVPQDFDNNSGYWNRIEMYCRELTERFEDVWVVS |
| | | GPLTLPQTRGDGKKIVSYQVIGEDNVAVPSHLYKVILARRSS |
| | | VSTEPLALGAFVVPNEAIGFQPQLTEFQVSLQDLEKLSGLVF |
| | | FPHLDRT |
| 102 | TENM1 | VTVSQMTSVLNGKTRRFADIQLQHGALCFNIRYGTTVEEEK |
| | | NHVLEIARQRAVAQAWTKEQRRLQEGEEGIRAWTEGEKQQ |
| | | LLSTGRVQGYDGYFVLSVEQYLELSDSANNIHFMRQSEIGR |
| | | R |
| 103 | TENM2 | TVSQPTLLVNGKTRRFTNIEFQYSTLLLSIRYGLTPDTLDEEK |
| | | ARVLDQARQRALGTAWAKEQQKARDGREGSRLWTEGEKQ |
| | | QLLSTGRVQGYEGYYVLPVEQYPELADSSSNIQFLRQNEMG |
| | | KR |
| 104 | RNAseK | MGWLRPGPRPLCPPARASWAFSHRFPSPLAPRRSPTPFFMAS |
| | | LLCCGPKLAACGIVLSAWGVIMLIMLGIFFNVHSAVLIEDVP |
| | | FTEKDFENGPQNIYNLYEQVSYNCFIAAGLYLLLGGFSFCQV |
| | | RLNKRKEYMVR |
| 105 | TALEN | MRIGKSSGWLNESVSLEYEHVSPPTRPRDTRRRPRAAGDGG |
| | | LAHLHRRLAVGYAEDTPRTEARSPAPRRPLPVAPASAPPAPS |
| | | LVPEPPMPVSLPAVSSPRFSAGSSAAITDPFPSLPPTPVLYAM |
| | | ARELEALSDATWQPAVPLPAEPPTDARRGNTVFDEASASSP |
| | | VIASACPQAFASPPRAPRSARARRARTGGDAWPAPTFLSRPS |
| | | SSRIGRDVFGKLVALGYSREQIRKLKQESLSEIAKYHTTLTG |
| | | QGFTHADICRISRRRQSLRVVARNYPELAAALPELTRAHIVD |
| | | IARQRSGDLALQALLPVATALTAAPLRLSASQIATVAQYGE |
| | | RPAIQALYRLRRKLTRAPLHLTPQQVVAIASNTGGKRALEA |
| | | VCVQLPVLRAAPYRLSTEQVVAIASNKGGKQALEAVKAHL |
| | | LDLLGAPYVLDTEQVVAIASHNGGKQALEAVKADLLDLRG |
| | | APYALSTEQVVAIASHNGGKQALEAVKADLLELRGAPYAL |
| | | STEQVVAIASHNGGKQALEAVKAHLLDLRGVPYALSTEQV |
| | | VAIASHNGGKQALEAVKAQLLDLRGAPYALSTAQVVAIAS |
| | | NGGGKQALEGIGEQLLKLRTAPYGLSTEQVVAIASHDGGKQ |
| | | ALEAVGAQLVALRAAPYALSTEQVVAIASNKGGKQALEAV |
| | | KAQLLELRGAPYALSTAQVVAIASHDGGNQALEAVGTQLV |
| | | ALRAAPYALSTEQVVAIASHDGGKQALEAVGAQLVALRAA |
| | | PYALNTEQVVAIASSHGGKQALEAVRALFPDLRAAPYALST |
| | | AQLVAIASNPGGKQALEAVRALFRELRAAPYALSTEQVVAI |
| | | ASNHGGKQALEAVRALFRGLRAAPYGLSTAQVVAIASSNG |
| | | GKQALEAVWALLPVLRATPYDLNTAQIVAIASHDGGKPAL |
| | | EAVWAKLPVLRGAPYALSTAQVVAIACISGQQALEAIEAHM |
| | | PTLRQASHSLSPERVAAIACIGGRSAVEAVRQGLPVKAIRRI |
| | | RREKAPVAGPPPASLGPTPQELVAVLHFFRAHQQPRQAFVD |
| | | ALAAFQATRPALLRLLSSVGVTEIEALGGTIPDATERWQRLL |
| | | GRLGFRPATGAAAPSPDSLQGFAQSLERTLGSPGMAGQSAC |
| | | SPHRKRPAETAIAPRSIRRSPNNAGQPSEPWPDQLAWLQRR |
| | | KRTARSHIRADSAASVPANLHLGTRAQFTPDRLRAEPGPIM |
| | | QAHTSPASVSFGSHVAFEPGLPDPGTPTSADLASFEAEPFGV |
| | | GPLDFHLDWLLQILET |
| 106 | TALEN | MDPIRSRTPSPARELLPGPQPDRVQPTADRGGAPPAGGPLDG |
| | | LPARRTMSRTRLPSPPAPSPAFSAGSFSDLLRQFDPSLLDTSL |
| | | LDSMPAVGTPHTAAAPAECDEVQSGLRAADDPPPTVRVAV |
| | | TAARPPRAKPAPRRRAAQPSDASPAAQVDLRTLGYSQQQQ |
| | | EKIKPKVGSTVAQHHEALVGHGFTHAHIVALSRHPAALGTV |
| | | AVKYQDMIAALPEATHEDIVGVGKQWSGARALEALLTVAG |
| | | ELRGPPLQLDTGQLVKIAKRGGVTAVEAVHASRNALTGAPL |
| | | NLTPAQVVAIASNNGGKQALETVQRLLPVLCQAHGLTPAQ |
| | | VVAIASHDGGKQALETMQRLLPVLCQAHGLPPDQVVAIAS |
| | | NIGGKQALETVQRLLPVLCQAHGLTPDQVVAIASHGGGKQ |
| | | ALETVQRLLPVLCQAHGLTPDQVVAIASHDGGKQALETVQ |
| | | RLLPVLCQAHGLTPDQVVAIASNGGGKQALETVQRLLPVLC |
| | | QAHGLTPDQVVAIASNGGKQALETVQRLLPVLCQAHGLTP |
| | | DQVVAIASHDGGKQALETVQRLLPVLCQTHGLTPAQVVAI |
| | | ASHDGGKQALETVQQLLPVLCQAHGLTPDQVVAIASNIGG |
| | | KQALATVQRLLPVLCQAHGLTPDQVVAIASNGGGKQALET |
| | | VQRLLPVLCQAHGLTPDQVVAIASNGGGKQALETVQRLLP |
| | | VLCQAHGLTQVQVVAIASNIGGKQALETVQRLLPVLCQAH |
| | | GLTPAQVVAIASHDGGKQALETVQRLLPVLCQAHGLTPDQ |
| | | VVAIASNGGGKQALETVQRLLPVLCQAHGLTQEQVVAIAS |
| | | NNGGKQALETVQRLLPVLCQAHGLTPDQVVAIASNGGGKQ |
| | | ALETVQRLLPVLCQAHGLTPAQVVAIASNIGGKQALETVQR |
| | | LLPVLCQDHGLTLAQVVAIASNIGGKQALETVQRLLPVLCQ |
| | | AHGLTQDQVVAIASNIGGKQALETVQRLLPVLCQDHGLTPD |
| | | QVVAIASNIGGKQALETVQRLLPVLCQDHGLTLDQVVAIAS |
| | | NGGKQALETVQRLLPVLCQDHGLTPDQVVAIASNSGGKQA |
| | | LETVQRLLPVLCQDHGLTPNQVVAIASNGGKQALESIVAQL |
| | | SRPDPALAALTNDHLVALACLGGRPAMDAVKKGLPHAPEL |
| | | IRRVNRRIGERTSHRVADYAQVVRVLEFFQCHSHPAYAFDE |
| | | AMTQFGMSRNGLVQLFRRVGVTELEARGGTLPPASQRWDR |
| | | ILQASGMKRAKPSPTSAQTPDQASLHAFADSLERDLDAPSP |
| | | MHEGDQTGASSRKRSRSDRAVTGPSAQHSFEVRVPEQRDA |
| | | LHLPLSWRVKRPRTRIGGGLPDPGTPIAADLAASSTVMWEQ |
| | | DAAPFAGAADDFPAFNEEELAWLMELLPQSGSVGGTI |
| 107 | ZNF638 | MSRPRFNPRGDFPLQRPRAPNPSGMRPPGPFMRPGSMGLPR |
| | | FYPAGRARGIPHRFAGHESYQNMGPQRMNVQVTQHRTDPR |
| | | LTKEKLDFHEAQQKKGKPHGSRWDDEPHISASVAVKQSSV |
| | | TQVTEQSPKVQSRYTKESASSILASFGLSNEDLEELSRYPDE |
| | | QLTPENMPLILRDIRMRKMGRRLPNLPSQSRNKETLGSEAVS |
| | | SNVIDYGHASKYGYTEDPLEVRIYDPEIPTDEVENEFQSQQN |
| | | ISASVPNPNVICNSMFPVEDVFRQMDFPGESSNNRSFFSVES |
| | | GTKMSGLHISGGQSVLEPIKSVNQSINQTVSQTMSQSLIPPS |
| | | MNQQPFSSELISSVSQQERIPHEPVINSSNVHVGSRGSKKNY |
| | | QSQADIPIRSPFGIVKASWLPKFSHADAQKMKRLPTPSMMN |
| | | DYYAASPRIFPHLCSLCNVECSHLKDWIQHQNTSTHIESCRQ |
| | | LRQQYPDWNPEILPSRRNEGNRKENETPRRRSHSPSPRRSRR |
| | | SSSSHRFRRSRSPMHYMYRPRSRSPRICHRFISRYRSRSRSRS |
| | | PYRIRNPFRGSPKCFRSVSPERMSRRSVRSSDRKKALEDVVQ |
| | | RSGHGTEFNKQKHLEAADKGHSPAQKPKTSSGTKPSVKPTS |
| | | ATKSDSNLGGHSIRCKSKNLEDDTLSECKQVSDKAVSLQRK |
| | | LRKEQSLHYGSVLLITELPEDGCTEEDVRKLFQPFGKVNDV |
| | | LIVPYRKEAYLEMEFKEAITAIMKYIETTPLTIKGKSVKICVP |
| | | GKKKAQNKEVKKKTLESKKVSASTLKRDADASKAVEIVTS |
| | | TSAAKTGQAKASVAKVNKSTGKSASSVKSVVTVAVKGNK |
| | | ASIKTAKSGGKKSLEAKKTGNVKNKDSNKPVTIPENSEIKTS |
| | | IEVKATENCAKEAISDAALEATENEPLNKETEEMCVMLVSN |
| | | LPNKGYSVEEVYDLAKPFGGLKDILILSSHKKAYIEINRKAA |
| | | ESMVKFYTCFPVLMDGNQLSISMAPENMNIKDEEAIFITLVK |
| | | ENDPEANIDTIYDRFVHLDNLPEDGLQCVLCVGLQFGKVDH |
| | | HVFISNRNKAILQLDSPESAQSMYSFLKQNPQNIGDHMLTCS |
| | | LSPKIDLPEVQIEHDPELEKESPGLKNSPIDESEVQTATDSPSV |
| | | KPNELEEESTPSIQTETLVQQEEPCEEEAEKATCDSDFAVETL |
| | | ELETQGEEVKEEIPLVASASVSIEQFTENAEECALNQQMENS |
| | | DLEKKGAEIINPKTALLPSDSVFAEERNLKGILEESPSEAEDFI |
| | | SGITQTMVEAVAEVEKNETVSEILPSTCIVTLVPGIPTGDEKT |
| | | VDKKNISEKKGNMDEKEEKEFNTKETRMDLQIGTEKAEKN |
| | | EGRMDAEKVEKMAAMKEKPAENTLFKAYPNKGVGQANKP |
| | | DETSKTSILAVSDVSSSKPSIKAVIVSSPKAKATVSKTENQKS |
| | | FPKSVPRDQINAEKKLSAKEFGLLKPTSARSGLAESSSKFKP |
| | | TQSSLTRGGSGRISALQGKLSKLDYRDITKQSQETEARPSIM |
| | | KRDDSNNKTLAEQNTKNPKSTTGRSSKSKEEPLFPFNLDEFV |
| | | TVDEVIEEVNPSQAKQNPLKGKRKETLKNVPFSELNLKKKK |
| | | GKTSTPRGVEGELSFVTLDEIGEEEDAAAHLAQALVTVDEVI |
| | | DEEELNMEEMVKNSNSLFTLDELIDQDDCISHSEPKDVTVLS |
| | | VAEEQDLLKQERLVTVDEIGEVEELPLNESADITFATLNTKG |
| | | NEGDTVRDSIGFISSQVPEDPSTLVTVDEIQDDSSDLHLVTLD |
| | | EVTEEDEDSLADFNNLKEELNFVTVDEVGEEEDGDNDLKV |
| | | ELAQSKNDHPTDKKGNRKKRAVDTKKTKLESLSQVGPVNE |
| | | NVMEEDLKTMIERHLTAKTPTKRVRIGKTLPSEKAVVTEPA |
| | | KGEEAFQMSEVDEESGLKDSEPERKRKKTEDSSSGKSVASD |
| | | VPEELDFLVPKAGFFCPICSLFYSGEKAMTNHCKSTRHKQN |
| | | TEKFMAKQRKEKEQNEAEERSSR |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-100. (canceled)

101. A synthetic RNA binding domain comprising an amino acid sequence with at least 90% sequence identity to SEQ ID NO: 6.

102. A polynucleotide sequence encoding the synthetic RNA binding domain of claim 101.

103. A vector comprising the polynucleotide sequence of claim 102.

104. The vector of claim 103, wherein the vector is a viral vector.

105. A pharmaceutical composition comprising the vector of claim 103 and a pharmaceutically acceptable excipient, carrier, or diluent.

106. A synthetic RNA binding domain comprising an amino acid sequence with at least 95% sequence identity to SEQ ID NO: 10.

107. A kit comprising the synthetic RNA binding domain of the claim 106.

108. A polynucleotide sequence encoding the synthetic RNA binding domain of claim 106.

109. A cell or cell culture expressing the polynucleotide sequence of claim 108.

110. A vector comprising the polynucleotide sequence of claim 108.

111. A method of delivering a synthetic site-specific RNA editing entity to a cell, comprising administering to the cell the vector of claim 110.

112. The method of claim 111, wherein the polynucleotide sequence is integrated into the genome of the cell.

113. A synthetic site-specific RNA editing entity targeting a pathogenic RNA that comprises a CAG repeat, the synthetic site-specific RNA editing entity comprising: (i) a synthetic RNA binding domain; and (ii) a cleavage domain; wherein the synthetic RNA binding domain comprises an amino acid sequence comprising (Cys/Ser/Asn)XxxXxxXxxGln that binds to adenine, wherein Xxx is any amino acid.

114. A method of treating a subject in need thereof, comprising administering to the subject the synthetic site-specific RNA editing entity of claim 113.

115. The method of claim 114, wherein the subject has a CAG repeat-associated disorder.

116. The method of claim 114, wherein the subject has Huntington's disease (HD), spinocerebellar ataxia (SCA), dentatorubral-pallidoluysian atrophy (DRPLA), or spinal and bulbar muscular atrophy (SBMA).

117. The method of claim 116, wherein the subject has the HD.

118. The method of claim 116, wherein the subject has the SCA.

119. The method of claim 118, wherein the subject has spinocerebellar ataxia (SCA) type 1, SCA type 2, SCA type 3, SCA type 6, SCA type 7, or SCA type 17.

120. The method of claim 119, wherein the subject has the SCA type 3.
